SEQUENCE LISTING (10 sequences)

SEQ ID NO: 1 - DNA, 23 bp, T7 phage
taatacgactcactatagggaga

SEQ ID NO: 2 - DNA, 23 bp, SP6 phage
  misc_feature (1)..(23): N equals unknown
atttaggtgacactatagaagng

SEQ ID NO: 3 - DNA, 23 bp, T3 phage
aattaaccctcactaaagggaga

SEQ ID NO: 4 - DNA, 51 bp, Artificial Sequence
  DNA/RNA Primer
aaacgacggccagtgaattgtaatacgactcactataggcgcdagagnnnn

SEQ ID NO: 5 - DNA, 32 bp, Artificial Sequence
  Primer
cccagaattcttttttttttttttttttttvn

SEQ ID NO: 6 - DNA, 19 bp, Artificial Sequence
  DNA/RNA Primer
gggcaattcaagcctannn

SEQ ID NO: 7 - DNA, 66 bp, Artificial Sequence
  Primer
ttttttttttttttttttttttttcgcggatatcactcagcataatgttaagtgaccggcagcaaa

SEQ ID NO: 8 - DNA, 17 bp, Artificial Sequence
  Primer
tatcaacgcagagtccc

SEQ ID NO: 9 - DNA, 18 bp, Artificial Sequence
  Primer
tttttttttttttttttt

SEQ ID NO: 10 - DNA, 51 bp, Artificial Sequence
  Primer
aaacgacggccagtgaattgtaatacgactcactataggcgcgagagcccc

(19) United States
(12) Patent Application Publication
(10) Pub. No.: US 2003/0186237 A1
(43) Pub. Date: Oct. 2, 2003

(54) Methods and compositions of amplifying RNA

(75) Inventors: Stephen D. Ginsberg, Houston, TX (US); Shaoli Che, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine

(21) Appl. No.: 10/075,335

(22) Filed: Feb. 14, 2002

Related U.S. Application Data
(60) Provisional application No. 60/268,664, filed on Feb. 14, 2001.
(60) Provisional application No. 60/348,242, filed on Nov. 7, 2001.
(60) Provisional application No. 60/268,645, filed on Feb. 14, 2001.
(60) Provisional application No. 60/344,557, filed on Nov. 7, 2001.
(60) Provisional application No. 60/306,216, filed on Jul. 18, 2001.
(60) Provisional application No. 60/350,176, filed on Nov. 9, 2001.

(51) Int. Cl. C12Q 1/68; C12P 19/34

(52) U.S. Cl. 435/6; 435/91.2

(57) ABSTRACT

The present invention pertains to a method that will increase the efficiency of second strand cDNA synthesis through a mechanism of “terminal continuation” before further RNA amplification by RNA transcription using, for example, a bacteriophage promoter. In a specific embodiment, a transcription promoter is attached to the 5′ region of cDNA through the same mechanism of “terminal continuation”. Genetic signals are subsequently amplified in a linear manner through RNA transcription. In specific embodiments, the orientation of the transcribed RNA is either sense or antisense, depending on the desired downstream application. In other embodiments, the present invention pertains to methods for extraction and amplification of RNA, particularly mRNA, from histologically stained tissues and cells.

The present invention claims priority to U.S. Ser. No. 60/268,664 entitled “A Novel Method to Amplify RNA” filed Feb. 14, 2001; U.S. Ser. No. 60/268,645 entitled “Detection of Gene Expression in Histologically Stained Tissues and Cells” filed Feb. 14, 2001; U.S. Ser. No. 60/306,216 entitled “Method and Composition of Amplifying mRNA through Terminal Continuation” filed Jul. 18, 2001; U.S. Ser. No. unknown entitled “RNA Amplification Method”, filed Nov. 7, 2001; U.S. Ser. No. unknown entitled “Detection of Gene Expression in Histologically Stained Tissues and Cells,” filed Nov. 7, 2001; and U.S. Ser. No. 60/350,176 entitled “Method and Composition of Amplifying Nucleic Acid through Terminal Continuation” filed Nov. 9, 2001; all of which are incorporated by reference herein in their entirety.

### FIELD OF THE INVENTION

The present invention is directed to methods to amplify a nucleic acid molecule, such as an RNA molecule. Specifically, the methods are directed to increasing the efficiency of second strand cDNA synthesis utilizing the mechanism of terminal continuation prior to further RNA amplification with an RNA polymerase. More specifically, the methods are directed to provide a double stranded (ds) cDNA molecule for in vitro transcription. In other embodiments, the present invention regards methods related to detection of gene expression, particularly from a histologically stained tissue.

### BACKGROUND OF THE INVENTION

Contemporary gene expression profiling or “molecular fingerprinting” is typically performed using cDNA array technology. Essentially, a gene array allows the investigation of multiple (e.g., hundreds to thousands) of genes simultaneously. However, fairly large quantities of tissues are needed for subsequent RNA extraction due to the lack of sensitivity of the methodology. The low sensitivity of methodology may be problematic in two aspects. First, the sources of tissues may be limited and, second, arrays can only be performed on a heterogeneous cell population since collection of large numbers of homogeneous tissues and/or cell types is often complicated.

Antisense RNA synthesis has been used to amplify genetic signals from limited amounts of tissues or cells (Van Gelder et al., 1990; Eberwine et al., 1992; U.S. Pat. No. 5,545,522). However, the antisense RNA synthesis method presently in use has a low efficiency in amplifying the genetic signals. Therefore, the overall sensitivity and reliability of the method is not optimal. The main obstacle for increasing the efficiency of the method is the problematic second strand cDNA synthesis. There are two procedures currently in use for second strand cDNA synthesis, self-priming and replacement synthesis. Self-priming uses the hairpin formed at the 3′ of first strand cDNA to self-prime the synthesis of second strand cDNA (Sambrook et al., 1989). However, the loop formed at the end has to be removed using S1 nuclease digestion. It is a poorly controlled reaction and invariably leads to the loss of the 5′ signal. In addition, self-priming can only be performed with Klenow fragment of E. coli DNA polymerase I, which is an enzyme with relatively low processivity. This factor further decreases the efficiency of the methodology. The replacement synthesis avoids S1 nuclease digestion altogether and has been used in RNA amplification. The reaction employs multiple enzymes, RNAse H, E. coli DNA polymerase I and bacteriophage T4 DNA ligase to digest RNA in a DNA:RNA complex, synthesize DNA fragments, and ligate them. In general, the reaction suffers from a low efficiency, likely caused by the multiple enzymatic steps involved. In summary, one key factor to increase of efficiency of RNA amplification is to increase the efficiency of second strand cDNA synthesis.

U.S. Pat. No. 5,545,522, Van Gelder et al. (1990), and Eberwine et al. (1992) are directed to synthesis of a cDNA from an RNA primed by a single complementary primer in the reaction, wherein the primer is linked to sequence of an RNA polymerase promoter region. Antisense RNA is transcribed from the cDNA by an RNA polymerase.

U.S. Pat. No. 5,962,272 regards preparing a DNA molecule using a template switching oligonucleotide. An RNA is contacted with a cDNA synthesis primer which anneals to the RNA, and the cDNA molecule is reverse transcribed to generate a mRNA-cDNA hybrid. A template switching oligonucleotide hybridizes to the 5′ CAP site and serves as a short, extended template for CAP-dependent extension of the 3′-end of the ss cDNA that is complementary to the template-switching oligonucleotide.

PCT application WO 00/75356 is directed to an RNA polymerase chain reaction wherein a poly (dT) primer primes a reverse transcription reaction to synthesize a first strand cDNA. The reaction is then followed by a terminal transferase tailing reaction to incorporate dGTPs to the 3′ end of the first strand cDNA, a second strand cDNA synthesis reaction, and transcription.

Furthermore, the functional state(s) of tissues and cells have been studied by morphological observation for over a century. The study of optimally prepared, i.e., fixed, sectioned, and/or stained tissues has long been a principal method for histological and histopathological investigation. Several histological staining methods were developed empirically on the basis of their capacity to increase the contrast of specific tissue constituents to enable the visualization of distinct cell types. Although histological stains are in most cases not specific to an individual cell type or protein, much information can be gleaned by utilizing classical histochemical preparations in conjunction with contemporary protein (e.g., immunocytochemistry) and molecular biological methodologies. However, the information gathered through morphological observation and molecular biological methods are often difficult to compare and correlate with each other. The problem arises mainly from the fact that the methods for morphology and molecular studies have been thought to be mutually exclusive. Thus, morphological observation and molecular procedures such as RNA amplification could not be performed on the same tissue section or cell. This limitation hinders direct examination, and ultimately, hypothesis testing, of the morphological features of tissues and distinct cell types with simultaneous examinations at a molecular level.

Contemporary gene expression profiling or “molecular fingerprinting” is typically performed using complementary deoxyribonucleic acid (cDNA) array technology. Essentially, a gene array allows the investigation of multiple (e.g., hundreds to thousands) of genes simultaneously. However, fairly large quantities of tissues are needed for subsequent RNA extraction due to the lack of sensitivity of the methodology. The low sensitivity of methodology may be problematic in two aspects. First, the sources of tissues may be limited and, second, arrays can only be performed on a heterogeneous cell population since collection of large numbers of homogeneous tissues and/or cell types is often complicated.

Reverse-transcriptase polymerase chain reaction (RT-PCR) has been the method of choice to amplify genetic signals when only limited starting materials are available. However, RT-PCR distorts the quantitative relationships between members of a gene population because it amplifies genes non-linearally (Phillips and Eberwine, 1996). As a result, PCR preferably amplifies abundant genes over rare genes and the weak signals of later populations may be further obscured by PCR amplification. Attempts to avoid this bias in PCR amplification include limiting the cycles of PCR. However, the amplification capacity of limited cycles of PCR reaction is greatly decreased. In vitro RNA transcription amplifies genes in a linear manner (Ginsberg et al., 1999; Ginsberg et al., 2000). Therefore, the original quantitative relationship of members in an amplified gene population is preserved. Amplified RNA is the method of choice for gene expression profiling when only a small quantity of starting material is available. The present invention describes a methodology that is useful for amplifying the genetic signals from histologically stained tissues and cells using the method of in vitro RNA transcription.

Saito et al. (1997) describe detection of RNA from liver tissue by extracting RNA from histologically stained sections, subjecting the RNA to strand-specific reverse transcription double PCR (Chu et al., 1994) and Southern blotting.

To et al. (1998) describe a technique to analyze mRNA from microdissected frozen tissue sections without RNA isolation. Lesions are microdissected from frozen tumor sections, sections are stained and immersed in a freezing solution, followed by RT-PCR analysis in the absence of further purification methods.

Florell et al. (2001) describe a protocol for preservation of RNA to maintain the integrity of tissue for pathologic diagnosis and to provide RNA for molecular analyses. Freshly excised tissue was treated with RNAlater™, a RNA storage solution, total RNA was extracted, followed by microarray analysis and northern analysis.

Thus, there is a void in the art using non-PCR-based methods to linearly amplify genetic signals from histologically stained tissues. The present invention is directed to provide methods and compositions for fulfilling such a void.

### SUMMARY OF THE INVENTION

The present invention describes a new procedure which results in the addition of a sequence complementary to an oligonucleotide to the 3′ region of a synthesized nucleic acid strand. This process is described as “terminal continuation”. The oligonucleotide used to add its complement to the 3′ region of the synthesized nucleic acid strand contains at least one specific nucleotide, preferably a guanine or deoxyguanine, or cytosine or deoxycytosine, at the 3′ end of the oligonucleotide. This oligonucleotide is described as the “terminal continuation oligonucleotide”. The complementary sequence of the oligonucleotide can be added to the 3′ end of the synthesized nucleic acid strand by a polymerase reaction using one primer and one terminal continuation oligonucleotide. One primer, the “first strand synthesis primer”, anneals to the 3′ end, or upstream of the 3′ end, of a target nucleic acid strand to initiate a polymerase-dependent synthesis of a nucleic acid strand, the “first strand nucleic acid”, that contains the complementary sequence of the target nucleic acid strand. The “terminal continuation oligonucleotide” is added so that a polymerase adds nucleotides complementary to the terminal continuation oligonucleotide at the 3′ end of the first strand nucleic acid synthesis reaction. As a result, second strand nucleic acid synthesis can be primed with the terminal continuation oligonucleotide or a part thereof. Thus, “terminal continuation” may add the complementary sequence of an oligonucleotide to the 3′ region of first strand nucleic acid, allowing the use of a primer comprising all or part of the oligonucleotide sequence for second strand synthesis.

A skilled artisan recognizes that by providing a known sequence at the 3′ region of first strand cDNA and a primer complementary to it, hairpin loops will not form, avoiding use of the destructive S1 nuclease digestion step associated with the “self-priming” method. Thus, the reaction of “terminal continuation” is highly efficient and offers improved sensitivity, as compared to the relatively low efficiency “self priming” or “replacement” synthesis of second strand cDNA. Furthermore, the synthesis of the second strand cDNA can be performed with robust enzymes such as Taq polymerase, which further improves the efficiency of the method.

When the target nucleic acid is RNA, the method of terminal continuation may incorporate the complementary sequence of a terminal continuation oligonucleotide to the 3′ end of a first strand nucleic acid which is cDNA. This may be achieved through the use of reverse transcriptase as the “polymerase”, a poly(dT) oligonucleotide as the “first strand synthesis primer”, and a terminal continuation oligonucleotide. In this embodiment, the sequence complementary to the terminal continuation oligonucleotide is incorporated to the 3′ end of first strand cDNA, where the sequence of first strand cDNA is the complementary sequence of the target RNA strand. The terminal continuation oligonucleotide may then be used as the primer to initiate second strand synthesis of cDNA through the use of a DNA polymerase.

Thus as described, the methods of the present invention are directed to the amplification of an RNA molecule. In a specific embodiment, the methods of the present invention increase the efficiency of second strand cDNA synthesis by utilizing the mechanism of terminal continuation prior to further RNA amplification with an RNA polymerase. In another specific embodiment, and in contrast to other methods known in the art, the methods are directed to provide a ds cDNA molecule for in vitro transcription. In an additional specific embodiment, and in contrast to other methods known in the art, the methods lack a terminal transferase tailing reaction and instead utilize an intrinsic activity of reverse transcriptase to incorporate deoxycytidine into the 3′ end of the first strand cDNA.

In addition, a transcription promoter such as an RNA synthesis promoter can be attached to the 5′ region of cDNA utilizing the same “terminal continuation” mechanism. That is, as the complementary sequence of the terminal continuation oligonucleotide is incorporated to the 3′ end of first strand cDNA, second strand cDNA synthesis, using the terminal continuation oligonucleotide containing the transcriptional promoter as a primer, results in a transcriptional promoter at the 5′ end of second strand cDNA. Therefore, in vitro transcription using this second strand cDNA as a template is possible, resulting in the RNA amplification of sense-strand RNA.

The orientation of RNAs subsequently transcribed and amplified will have an orientation of either “sense” or “antisense” direction depending on which strand a promoter is attached to. This may be accomplished by designing the terminal continuation oligonucleotide to possess a transcriptional promoter, and to design the first strand cDNA synthesis primer with a different transcriptional promoter. Compared to the 3′-promoter attachment, the RNA synthesized from a 5′ promoter avoids the shortcomings of antisense RNA synthesis presently in use and preferentially preserves the 5′ sequence of mRNAs. This advantage is even more significant when more than one round of amplification is needed. Furthermore, sense RNA can be used as a protein translation template, providing an additional powerful methodology for downstream proteomic investigations.

The present invention provides a highly efficient means for the synthesis of second strand cDNA by providing a sequence-specific priming method. The RNA amplification is subsequently performed by RNA transcription driven by a bacteriophage promoter attached to cDNA. Using this methodology, even a small amount of starting RNA will be amplified linearly, and can be utilized for many downstream applications. The downstream applications of amplified RNA include, but are not restricted to, gene expression profiling, cDNA microarray analysis, cDNA library construction, and subtraction library construction following the conversion of amplified RNA to double stranded cDNA. The synthesized sense RNA of a total starting mRNA population can also be used as template for in vitro protein translations. A variety of reagent kits for the procedures are developed as a result of, and are inclusive under, the present invention.

Another obstacle to increase the sensitivity of current RNA amplification method is the location of the RNA synthesis promoter. A critical component of the method, the bacteriophage transcriptional promoter, is attached to the 3′ end of, for example, a mRNA through a primer comprising of a DNA sequence complementary to poly(A+) sequence of mRNA and a promoter. The subsequent amplification step amplifies the 3′ sequence, whereas the informative protein coding sequence tends to be localized to the 5′ regions of mRNAs. However, the sensitivity of the method is an improvement on other known methods, reducing the loss of informative protein coding sequence.

The reaction of “terminal continuation” is highly efficient. The method, when used in conjunction with RNA amplification, offers improved sensitivity as compared to the relatively inefficient “replacement” synthesis of second strand cDNA synthesis. Furthermore, the synthesis of the second strand cDNA can be performed with any robust DNA polymerase, further improving the efficiency of the method.

Furthermore, this invention further produces multiple experimental advantages over known methods in the art, including: 1). Providing a suitable platform for the correlation between morphology and “molecular fingerprinting”, thus facilitating direct comparison and evaluation of disease states and genetic alterations; 2). Only limited target tissues or cells from a wide variety of sources (for example, but not limited to, fresh tissues and archival paraffin-embedded tissues) are needed. Thus, it is possible to study gene expression in a homogeneous cell population, even a single cell (Ginsberg et al., 1999; Ginsberg et al., 2000); 3). Gene expression levels can be investigated from tissue sections used for diagnostic purposes; 4). When utilized in combination with other molecular methods, such as library construction and/or recombinant protein expression, the applicability can be further extended to subtractive hybridization, cloning of novel gene targets, and ultimately, generating probes and expression of recombinant proteins.

A skilled artisan recognizes, based on the methods and compositions described herein, that the amplification of the RNA from the histologically stained tissue does not include polymerase chain reaction. Specifically, the genetic signals are amplified through RNA synthesis by in vitro transcription, a method distinct from polymerase chain reaction.

An object of the present invention is a method to amplify an RNA molecule, comprising obtaining the RNA molecule; introducing to the mRNA molecule a first primer, wherein the first primer comprises a region that hybridizes under suitable conditions to a complementary region of the RNA molecule; introducing to the RNA molecule and the first primer a second primer, wherein the second primer comprises at least one riboguanine at the 3′ end of the primer; synthesizing a first complementary nucleic acid molecule to the RNA molecule by extending the first primer using reverse transcriptase under conditions wherein the synthesis results in there being more than one cytosine at the 3′ end of the first complementary nucleic acid molecule, wherein the synthesis results in an RNA-first complementary nucleic acid molecule hybrid comprising the first primer and its extension product bound to the second primer and the RNA; removing the RNA molecule and the second primer from the hybrid; synthesizing a second complementary nucleic acid molecule to the first complementary nucleic acid molecule, wherein the synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and transcribing at least one mRNA molecule from the first complementary nucleic acid molecule and second complementary nucleic acid molecule hybrid. In a specific embodiment, the RNA molecule is an mRNA molecule. In a specific embodiment, the RNA is a tRNA molecule. In another specific embodiment, the RNA is a rRNA molecule. In an additional specific embodiment, the RNA molecule is obtained from a plurality of RNA molecules. In another specific embodiment, the plurality of RNA molecules comprises mRNA, tRNA, rRNA, or a combination thereof. In an additional specific embodiment, the first primer further comprises a region comprising at least two poly(dT)s. In another specific embodiment, the first primer is a short primer of random sequence. In a further specific embodiment, the first primer further comprises a region selected from the group consisting of a promoter region, a restriction enzyme digestion sequence, and a combination thereof. In another specific embodiment, the first primer further comprises a promoter region. In an additional specific embodiment, the promoter is a bacteriophage transcription promoter. In another specific embodiment, the bacteriophage transcription promoter is selected from the group consisting of T7 RNA polymerase promoter, T3 RNA polymerase promoter, SP6 RNA polymerase promoter, and a recombinant promoter. In another specific embodiment, the second primer comprises a random sequence at it 5′ end and at least one riboguanine at its 3′ end. In another specific embodiment, the second primer further comprises a region selected from the group consisting of a promoter region, a protein translation start region, a restriction enzyme digestion sequence, and a combination thereof. In an additional specific embodiment, the second primer further comprises a promoter. In another specific embodiment, the promoter is a bacteriophage transcription promoter. In a further specific embodiment, the bacteriophage transcription promoter is selected from the group consisting of T7 RNA polymerase promoter, T3 RNA polymerase promoter, SP6 RNA polymerase promoter, and a recombinant promoter. In a further specific embodiment, the reverse transcriptase is selected from the group consisting of Taq reverse transcriptase, Moloney Murine Leukemia Virus reverse transcriptase, Moloney Murine Leukemia Virus reverse transcriptase lacking RNAseH activity, Avian Myeloblastosis Virus reverse transcriptase, Avian Myeloblastosis Virus reverse transcriptase lacking RNAseH activity, human T-cell leukemia virus type I (HTLV-I), Rous-associated virus 2 (RAV2), bovine leukemia virus (BLV), Rous sarcoma virus (RSV), HIV-1 reverse transcriptase, TERT reverse transcriptase, and Tth reverse transcriptase. In another specific embodiment, the method further comprises at least one step of reverse transcribing the mRNA molecule from the transcription step, wherein the reverse transcription results in generating at least one cDNA molecule. In an additional specific embodiment, the reverse transcribing step is primed by at least one random primer. In another specific embodiment, the reverse transcribing step is primed by a primer attached to the first complementary nucleic acid molecule, the second complementary nucleic acid molecule, or a combination thereof. In an additional specific embodiment, the cDNA molecule comprises at least one promoter sequence. In another specific embodiment, the promoter is a bacteriophage transcription promoter. In a specific embodiment, the bacteriophage transcription promoter is selected from the group consisting of T7 RNA polymerase promoter, T3 RNA polymerase promoter, SP6 RNA polymerase promoter, and a recombinant promoter. In a further specific embodiment, the RNA is removed by RNAase digestion. In an additional specific embodiment, the RNA is removed by RNAse digestion, by heating in solution comprising a low concentration of MgCl₂, or by a combination thereof.

In another embodiment of the present invention, there is a method to amplify an mRNA molecule, comprising obtaining the mRNA molecule; introducing to the mRNA molecule a first primer, wherein the first primer comprises at least two poly(dT)s; and random sequences; introducing to the mRNA molecule and the first primer a second primer, wherein the second primer comprises at least one riboguanine at the 3′ end of the primer; and a bacteriophage promoter sequence; synthesizing a first complementary nucleic acid molecule to the mRNA molecule by extending the first primer using reverse transcriptase under conditions wherein the synthesis results in there being more than one cytosine at the 3′ end of the first complementary nucleic acid molecule, wherein the synthesis results in an mRNA-first complementary nucleic acid molecule hybrid comprising the first primer and its extension product bound to the second primer and the mRNA; removing the mRNA molecule and the second primer from the hybrid; synthesizing a second complementary nucleic acid molecule to the first complementary nucleic acid molecule, wherein the synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and transcribing at least one mRNA molecule from the first complementary nucleic acid molecule and second complementary nucleic acid molecule hybrid.

In another embodiment of the present invention there is a method to amplify an mRNA molecule, comprising obtaining the mRNA molecule; introducing to the mRNA molecule a first primer, wherein the first primer comprises at least two poly(dT)s; and

a bacteriophage promoter sequence; introducing to the mRNA molecule and the first primer a second primer, wherein the second primer comprises at least one riboguanine at the 3′ end of the primer; synthesizing a first complementary nucleic acid molecule to the mRNA molecule by extending the first primer using reverse transcriptase under conditions wherein the synthesis results in there being more than one cytosine at the 3′ end of the first complementary nucleic acid molecule, wherein the synthesis results in an mRNA-first complementary nucleic acid molecule hybrid comprising the first primer and its extension product bound to the second primer and the mRNA; removing the mRNA molecule and the second primer from the hybrid; introducing to the complementary nucleic acid molecule an oligo (dNTP) primer with substantially the same sequence as the second primer; synthesizing a second complementary nucleic acid molecule to the first complementary nucleic acid molecule, wherein the synthesis results in a first complementary nucleic acid molecule and second complementary nucleic acid molecule hybrid; and transcribing at least one mRNA molecule from the first complementary nucleic acid molecule and second complementary nucleic acid molecule hybrid, wherein the at least one mRNA molecule is an antisense mRNA.

In an additional embodiment of the present invention, there is a method to amplify an mRNA molecule, comprising obtaining the mRNA molecule; introducing to the mRNA molecule a first primer, wherein the first primer comprises at least two poly(dT)s or a short primer of random sequence; introducing to the mRNA molecule and the first primer a second primer, wherein the second primer comprises at least one riboguanine at the 3′ end of the primer; and a bacteriophage promoter sequence; synthesizing a first complementary nucleic acid molecule to the mRNA molecule by extending the first primer using reverse transcriptase under conditions wherein the synthesis results in there being more than one cytosine at the 3′ end of the first complementary nucleic acid molecule, wherein the synthesis results in an mRNA-first complementary nucleic acid molecule hybrid comprising the first primer and its extension product bound to the second primer and the mRNA; removing the mRNA molecule and the second primer from the hybrid; introducing to the complementary nucleic acid molecule an oligo (dNTP) primer with substantially the same sequence as the second primer; synthesizing a second complementary nucleic acid molecule to the first complementary nucleic acid molecule, wherein the synthesis results in a first complementary nucleic acid molecule and second complementary nucleic acid molecule hybrid; and transcribing at least one mRNA molecule from the first complementary nucleic acid molecule and second complementary nucleic acid molecule hybrid, wherein the at least one mRNA molecule is a sense mRNA molecule.

In another embodiment of the present invention there is a kit for amplifying an RNA molecule using the method of claim 1, wherein the kit is in a suitable container and comprises the first primer, the second primer, the third primer, or a combination thereof. In a specific embodiment, the first primer is a short primer of random sequences. In another specific embodiment, the first primer further comprises a region selected from the group consisting of a promoter, a restriction enzyme digestion sequence, and a combination thereof. In another specific embodiment, the second primer further comprises a region selected from the group consisting of a promoter, a restriction enzyme digestion sequence, and a combination thereof.

In an additional embodiment of the present invention, there is a method of providing a substrate for in vitro transcription, comprising obtaining the mRNA molecule; introducing to the mRNA molecule a first primer, wherein the first primer comprises a region which anneals under suitable conditions to a complementary region of the mRNA molecule; introducing to the mRNA molecule and the first primer a second primer, wherein the second primer comprises at least one riboguanine at the 3′ end of the primer; synthesizing a first complementary nucleic acid molecule to the mRNA molecule by extending the first primer using reverse transcriptase under conditions wherein the synthesis results in there being more than one cytosine at the 3′ end of the first complementary nucleic acid molecule, wherein the synthesis results in an mRNA-first complementary nucleic acid molecule hybrid comprising the first primer and its extension product bound to the second primer and the mRNA; removing the mRNA molecule and the second primer from the hybrid; synthesizing a second complementary nucleic acid molecule to the first complementary nucleic acid molecule, wherein the synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and transcribing at least one mRNA molecule from the first complementary nucleic acid molecule and second complementary nucleic acid molecule hybrid.

In an embodiment of the present invention, there is a method to amplify an RNA molecule, comprising obtaining said RNA molecule; introducing to said mRNA molecule a first primer, wherein said first primer comprises a region that hybridizes under suitable conditions to a complementary region of said RNA molecule; introducing to said RNA molecule and said first primer a second primer, wherein said second primer comprises at least one riboguanine at the 3′ end of said primer; synthesizing a first complementary nucleic acid molecule to said RNA molecule by extending said first primer using reverse transcriptase under conditions wherein said synthesis results in there being more than one cytosine at the 3′ end of said first complementary nucleic acid molecule, wherein said synthesis results in an RNA-first complementary nucleic acid molecule hybrid comprising the first primer and its extension product bound to the second primer and the RNA; removing said RNA molecule and said second primer from said hybrid; synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and transcribing at least one mRNA molecule from said first complementary nucleic acid molecule and second complementary nucleic acid molecule hybrid. In a specific embodiment, the RNA molecule is an mRNA molecule, a tRNA molecule, or a rRNA molecule. In another specific embodiment, the RNA molecule is obtained from a plurality of RNA molecules. In a further specific embodiment, the plurality of RNA molecules comprises mRNA, tRNA, rRNA, or a combination thereof. In an additional specific embodiment, the first primer further comprises a region comprising at least two poly(dT)s. In an additional specific embodiment, the first primer is a short primer of random sequence. In an additional specific embodiment, the first primer farther comprises a region selected from the group consisting of a promoter region, a restriction enzyme digestion sequence, and a combination thereof. In a further specific embodiment, the first primer further comprises a promoter region. In another specific embodiment, the promoter is a bacteriophage transcription promoter. In an additional specific embodiment, the bacteriophage transcription promoter is selected from the group consisting of T7 RNA polymerase promoter, T3 RNA polymerase promoter, SP6 RNA polymerase promoter, and a recombinant promoter. In another specific embodiment, the second primer comprises a random sequence at it 5′ end and at least one riboguanine at its 3′ end. In a further specific embodiment, the second primer further comprises a region selected from the group consisting of a promoter region, a protein translation start region, a restriction enzyme digestion sequence, and a combination thereof. In an additional specific embodiment, the second primer further comprises a promoter. In another specific embodiment, the promoter is a bacteriophage transcription promoter. In a further specific embodiment, the bacteriophage transcription promoter is selected from the group consisting of T7 RNA polymerase promoter, T3 RNA polymerase promoter, SP6 RNA polymerase promoter, and a recombinant promoter. In a specific embodiment, the reverse transcriptase is selected from the group consisting of Taq reverse transcriptase, Moloney Murine Leukemia Virus reverse transcriptase, Moloney Murine Leukemia Virus reverse transcriptase lacking RNAseH activity, Avian Myeloblastosis Virus reverse transcriptase, Avian Myeloblastosis Virus reverse transcriptase lacking RNAseH activity, human T-cell leukemia virus type I (HTLV-I), Rous-associated virus 2 (RAV2), bovine leukemia virus (BLV), Rous sarcoma virus (RSV), HIV-1 reverse transcriptase, TERT reverse transcriptase, and Tth reverse transcriptase. In another specific embodiment, the method further comprises at least one step of reverse transcribing said mRNA molecule from said transcription step, wherein said reverse transcription results in generating at least one cDNA molecule. In an additional specific embodiment, the reverse transcribing step is primed by at least one random primer. In a further specific embodiment, the reverse transcribing step is primed by a primer attached to said first complementary nucleic acid molecule, said second complementary nucleic acid molecule, or a combination thereof. In another specific embodiment, the cDNA molecule comprises at least one promoter sequence. In a further specific embodiment, the promoter is a bacteriophage transcription promoter. In an additional specific embodiment, the bacteriophage transcription promoter is selected from the group consisting of T7 RNA polymerase promoter, T3 RNA polymerase promoter, SP6 RNA polymerase promoter, and a recombinant promoter. In another specific embodiment, the RNA is removed by RNAase digestion. In a further specific embodiment, the RNA is removed by RNAse digestion, by heating in solution comprising a low concentration of MgCl₂, or by a combination thereof.

In an embodiment of the present invention, there is a method to amplify an mRNA molecule, comprising obtaining said mRNA molecule; introducing to said mRNA molecule a first primer, wherein said first primer comprises at least two poly(dT)s; and random sequences; introducing to said mRNA molecule and said first primer a second primer, wherein said second primer comprises at least one riboguanine at the 3′ end of said primer; and a bacteriophage promoter sequence; synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA; removing said mRNA molecule and said second primer from said hybrid; synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and transcribing at least one mRNA molecule from said first complementary nucleic acid molecule and second complementary nucleic acid molecule hybrid.

In another embodiment of the present invention, there is a method to amplify an mRNA molecule, comprising obtaining said mRNA molecule; introducing to said mRNA molecule a first primer, wherein said first primer comprises at least two poly(dT)s; and a bacteriophage promoter sequence; introducing to said mRNA molecule and said first primer a second primer, wherein said second primer comprises at least one riboguanine at the 3′ end of said primer; synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA; removing said mRNA molecule and said second primer from said hybrid; introducing to said complementary nucleic acid molecule an oligo (dNTP) primer with substantially the same sequence as said second primer; synthesizing a second complementary nucleic acid molecule to said first complementary nucleic acid molecule, wherein said synthesis results in a first complementary nucleic acid molecule and second complementary nucleic acid molecule hybrid; and transcribing at least one mRNA molecule from said first complementary nucleic acid molecule and second complementary nucleic acid molecule hybrid, wherein said at least one mRNA molecule is an antisense mRNA.

In an additional embodiment of the present invention, there is a method to amplify an mRNA molecule, comprising obtaining said mRNA molecule; introducing to said mRNA molecule a first primer, wherein said first primer comprises at least two poly(dT)s or a short primer of random sequence; introducing to said mRNA molecule and said first primer a second primer, wherein said second primer comprises: at least one riboguanine at the 3′ end of said primer; and a bacteriophage promoter sequence; synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA; removing said mRNA molecule and said second primer from said hybrid; introducing to said complementary nucleic acid molecule an oligo (dNTP) primer with substantially the same sequence as said second primer; synthesizing a second complementary nucleic acid molecule to said first complementary nucleic acid molecule, wherein said synthesis results in a first complementary nucleic acid molecule and second complementary nucleic acid molecule hybrid; and transcribing at least one mRNA molecule from said first complementary nucleic acid molecule and second complementary nucleic acid molecule hybrid, wherein said at least one mRNA molecule is a sense mRNA molecule.

In an additional embodiment of the present invention, there is a kit for amplifying an RNA molecule using the method of claim 1, wherein said kit is in a suitable container and comprises said first primer, said second primer, said third primer, or a combination thereof. In a specific embodiment, the first primer is a short primer of random sequences. In another specific embodiment, the first primer further comprises a region selected from the group consisting of a promoter, a restriction enzyme digestion sequence, and a combination thereof. In a further specific embodiment, the second primer further comprises a region selected from the group consisting of a promoter, a restriction enzyme digestion sequence, and a combination thereof.

In an additional embodiment of the present invention, there is a method of providing a substrate for in vitro transcription, comprising obtaining said mRNA molecule; introducing to said mRNA molecule a first primer, wherein said first primer comprises a region which anneals under suitable conditions to a complementary region of said mRNA molecule; introducing to said mRNA molecule and said first primer a second primer, wherein said second primer comprises at least one riboguanine at the 3′ end of said primer; synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA; removing said mRNA molecule and said second primer from said hybrid; synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and transcribing at least one mRNA molecule from said first complementary nucleic acid molecule and second complementary nucleic acid molecule hybrid.

In another embodiment of the present invention, there is a method of detecting an RNA from a histologically-stained cell, comprising obtaining the cell; extracting RNA from the cell; and amplifying the RNA. In a specific embodiment, the cell is in a tissue.

In another embodiment of the present invention, there is a method of detecting an RNA from a cell, comprising obtaining the cell; histologically staining the cell; extracting RNA from the cell; and amplifying the RNA. In a specific embodiment, the cell is in a tissue. In a further specific embodiment, the tissue is fresh tissue or fixed tissue. In another specific embodiment, the tissue is fixed by acetone, aldehyde derivatives, ethanol, or combinations thereof. In a specific embodiment, the cell is from a physiological body fluid, a pathological exudate, or a pathological transudate. In a further specific embodiment, the physiological body fluid is blood, cerebrospinal fluid, urine, sweat, semen, or saliva. In an additional specific embodiment, the cells are in blood, bone marrow, cerebrospinal fluid, or any other physiological body fluids or any pathological exudates or transudates. In a further specific embodiment, the cell is from bone marrow. In an additional specific embodiment, the cell is from in vitro cultured cells. In another specific embodiment, the histological stain identifies cellular structures. In a further specific embodiment, the cellular structures are mitochondria, centrioles, rough endoplasmic reticulum, smooth endoplasmic reticulum, peroxisomes, endosomes, lysosomes, vesicles, Golgi apparatus, nucleus, cytoplasm, or a combination thereof. In a further specific embodiment, the histological stain identifies tissue structures. In an additional specific embodiment, the tissue structures are structures of lamina, matrix, or a combination thereof. In a further specific embodiment, the histological stain is Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In a specific embodiment, the extracting step further comprises dissection of the cell from the tissue. In a specific embodiment, the dissection is from a micropipette on a micromanipulator or by laser capture microdissection. In a further specific embodiment, the amplifying step further comprises synthesis of cDNA from the RNA. In a specific embodiment, the synthesis of cDNA further comprises synthesizing the cDNA by reverse transcriptase with an oligonucleotide that binds the RNA. In an additional specific embodiment, the RNA amplification method is in vitro transcription. In a further specific embodiment, the amplification is by a method which comprises introducing to said RNA molecule a first primer, wherein said first primer comprises a region that hybridizes under suitable conditions to a complementary region of said RNA molecule; introducing to said RNA molecule and said first primer a second primer, wherein said second primer comprises at least one riboguanine at the 3′ end of said primer; synthesizing a first complementary nucleic acid molecule to said RNA molecule by extending said first primer using reverse transcriptase under conditions wherein said synthesis results in there being more than one cytosine at the 3′ end of said first complementary nucleic acid molecule, wherein said synthesis results in an RNA-first complementary nucleic acid molecule hybrid comprising the first primer and its extension product bound to the second primer and the RNA; removing said RNA molecule and said second primer from said hybrid; synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and transcribing at least one mRNA molecule from said first complementary nucleic acid molecule and second complementary nucleic acid molecule hybrid.

A kit, housed in a suitable container, for the detection of RNA from a cell in a histologically-stained tissue, comprising dye/histological stain, RNA extraction reagent, RNA precipitation carrier, oligo (dT) primer, reverse transcriptase, DNA polymerase, RNA polymerase, RNAse inactivating agent, terminal continuation oligonucleotide, dNTPs, NTPs, or a combination thereof. In a specific embodiment, the RNA polymerase is T7 RNA polymerase, T3 RNA polymerase, or SP6 RNA polymerase. In a further specific embodiment, the kit further comprises a vector, a ligase, or a combination thereof. In an additional specific embodiment, the dye/histological stain is Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocarmine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffinan's violet, Imperial red, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulfate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In an embodiment of the present invention, there is a method of incorporating a nucleic acid sequence to a 3′ region of a synthesized nucleic acid strand comprising incubating a target nucleic acid strand with a terminal continuation oligonucleotide, and a first strand synthesis primer which is complementary to a region at the 3′ end or a region upstream of the 3′ end of the target nucleic acid strand under conditions that facilitate hybridization of the first strand synthesis primer to the target nucleic acid strand; and extending the primer, wherein the extending is carried out with a polymerase such that extension synthesizes a nucleic acid strand comprising the first strand synthesis primer, a complementary sequence of the target nucleic acid strand, and a complement of the terminal continuation oligonucleotide. In a specific embodiment, the terminal continuation oligonucleotide contains at least one guanine, deoxyguanine, cytosine, or deoxycytosine at the 3′ end of the terminal continuation oligonucleotide. In a further specific embodiment, the target nucleic acid strand is RNA and the polymerase is reverse-transcriptase, such that the nucleic acid synthesized in the extending step is a first strand cDNA comprising the first strand synthesis primer, a complement of the target nucleic acid strand, and a complement of the terminal continuation oligonucleotide at the 3′ end. In a specific embodiment, the RNA is mRNA. In another specific embodiment, the first strand synthesis primer comprises at least two thymidine residues at its 3′ end. In a further specific embodiment, the first strand synthesis primer comprises a random hexamer sequence of nucleic acid. In another specific embodiment, the terminal continuation oligonucleotide comprises at least two nucleotides selected from a group consisting of guanine, deoxyguanine, cytosine or deoxycytosine bases. In a further specific embodiment, the mehod further comprises the additional steps incubating the first strand cDNA with the terminal continuation oligonucleotide under conditions that facilitate hybridization of the terminal continuation oligonucleotide to the first strand cDNA; and extending the terminal continuation oligonucleotide, wherein said extending is carried out with a DNA polymerase such that extension synthesizes a second strand cDNA comprising the sequence of the terminal continuation oligonucleotide and a complementary sequence of the first strand cDNA. In a specific embodiment, the DNA polymerase is Taq polymerase. In another specific embodiment, the first strand synthesis primer comprises a transcriptional promoter sequence. In an additional specific embodiment, the terminal continuation oligonucleotide comprises a transcriptional promoter sequence and at least one guanine, deoxyguanine, cytosine, or deoxycytosine at the 3′ end of the terminal continuation oligonucleotide. In an additional specific embodiment, the terminal continuation oligonucleotide comprises a transcriptional promoter sequence and at least one guanine or cytosine at the 3′ end of the terminal continuation oligonucleotide. In a further specific embodiment, the method comprises the additional steps incubating the second strand cDNA with a RNA polymerase capable of binding to the transcriptional promoter sequence; and transcribing the second strand cDNA wherein the transcribing synthesizes a RNA transcript complementary in sequence to the second strand cDNA.

In another specific embodiment, the method further comprises the additional steps incubating the first strand cDNA with a RNA polymerase capable of binding to the transcriptional promoter sequence; and transcribing the first strand cDNA wherein the transcribing synthesizes a RNA transcript complementary in sequence to the first strand cDNA. In a specific embodiment, the first strand synthesis primer comprises a transcriptional promoter sequence and wherein the terminal continuation oligonucleotide comprises at least one guanine, deoxyguanine, cytosine, or deoxycytosine at its 3′ end and a transcriptional promoter sequence different from the transcriptional promoter sequence in the first strand synthesis primer. In a specific embodiment, the method further comprises the additional steps incubating the first strand cDNA with a RNA polymerase capable of binding to the transcriptional promoter sequence located on the first strand cDNA; transcribing the first strand cDNA wherein the transcribing synthesizes a RNA transcript complementary in sequence to the first strand cDNA; incubating the second cDNA strand with a RNA polymerase capable of binding to the transcriptional promoter sequence located on the second strand cDNA; and transcribing the second strand cDNA wherein the transcribing synthesizes a RNA transcript complementary in sequence to the second strand cDNA. In a specific embodiment, the synthesized RNA transcripts are used as templates for in vitro translation.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 is a schematic summary of the method of the present invention demonstrating attachment of a T7 promoter to the 3′ region of mRNA and the mechanism of terminal continuation.

FIG. 2 is a schematic summary of the method of the present invention demonstrating attachment of a T7 promoter to the 5′ region of mRNA and the mechanism of terminal continuation.

FIG. 3 is a schematic summary of the method of the present invention demonstrating attachment of a T7 promoter to the 5′ region and a SP6 promoter to the 3′ region of mRNA and the mechanism of terminal continuation.

FIG. 4 shows a diagram of RNA amplification based cDNA library construction.

FIG. 5 illustrates a schematic summary of the method regarding detection of RNA from a histologically stained sample.

FIG. 6 shows microdissection of cells from tissue sections. Individual cells are microdissected with a micropipette under the guidance of a micromanipulator. The cell can be physically attached to the tip of the micropipette (as shown in this schematic) or aspirated into the fluid-filled pipette tip. Laser capture microdissection can also be used to isolate one or more cells from tissue sections adhered to glass slides or coverslips.

FIG. 7 demonstrates expression profiles of normal (NCI) and Alzheimer's diseased (AD) tissues using methods of the present invention.

FIG. 8 shows amplification and detection of various genes of two adjacent regions from the same tissue by present method versus aRNA method in the art. The relative hybridization signal intensity of the low, moderate, and higher expressing genes using the new methodology of present invention are improved compared to aRNA method known in the art.

FIGS. 9A through 9C show the methods of the present invention. FIGS. 9A and 9B schematically illustrate the method. FIG. 9C demonstrates robust linear amplification.

FIGS. 10A through 10C demonstrate amplification with the methods of the present invention. FIG. 10A utilizes biological samples of RNA extracted from a variety of brain sources including post morten hippocampus and basal forebrain. FIG. 10B shows a comparison of different extraction methods. FIG. 10C shows a scatter plot demonstrating a linear relationship between TC RNA input concentration and mean hybridization signal intensity of all cDNA clones and an individual clone (CREB) on a custom-designed cDNA array.

FIGS. 11A and 11B demonstrates that methods of the present invention has increased sensitivity for the threshold of detection of genes with low hybridization signal intensity. FIG. 11A demonstrates a dot blot assay showing increased sensitivity for genes with relative low abundance. FIG. 11B shows a quantitation in total, normalized hybridization signal intensity for custom-designed cDNA array.

FIG. 12 presents a microscopic field during the microdissection of mouse dentate gyrus granule cells described in Example 1. Arrows in frames B & C show the aspiration device removing a single cell.

FIG. 13 presents microarray expression data of Example 8. The top panel shows representative raw microarray data of mRNA expression of GluR1, R2, R3, R4, R6 and R7 genes. Vehicle is a negative control experiment, and KA 1 DPL and KA 5DPL are two different experiments using intracerebral injection of kainate. The bottom panels show the average of mRNA expression levels from multiple experiments.

FIG. 14 presents microarray expression data of Example 9. The top panel shows representative microarray data of mRNA expression of synaptic marker genes from neurons of subjects with either no cognitive impairment (NCI) or Alzheimer's disease (AD). The bottom panel shows the average mRNA expression levels for these genes from multiple experiments.

FIG. 15 presents a schematic of the instrument used for LCM. In section A, cells are identified for isolation through microscopy. These targeted cells are then primed for separation from tissue by an ultraviolet or infrared laser beam. A transfer film attached to either a microfuge cap or membrane adheres the cell(s) of interest for removal. The microfuge cap or membrane containing the cell(s) of interest is then removed from the instrument. Section B shows the part of the apparatus that is responsible for the transfer of cells.

FIG. 16 depicts a comparison of methods of the present invention with different histochemical stains from adjacent tissue sections.

FIG. 17 is a quantitative analysis using methods of the present invention for total signal intensity from adjacent sections stained with an antibody (neurofilament) and histologically (cresyl violet).

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THEMENTION

As used herein the specification, “a” or “an” may mean one or more. As used herein in the claim(s), when used in conjunction with the word “comprising”, the words “a” or “an” may mean one or more than one. As used herein “another” may mean at least a second or more.

I. Definitions

The term “histologically-stained tissue” as used herein is defined as tissue sections or cells stained by any of a great variety of combinations of dyes that color various constituents more or less selectively, or the application to histological preparations of physical and chemical methods of analysis that permit identification of chemical substances in their normal sites in tissues.

The term “in vitro transcription” as used herein is defined as generation of an RNA molecule from a DNA template under conditions outside of a living cell.

The term “laser capture microdissection” as used herein is defined as the use of an infrared (IR) laser beam to remove a desired cell from a nondesired cell. In preferred embodiments, the desired cell is a cancer cell and the nondesired cell is a normal cell.

The term “oligonucleotides” as used herein are short-length, single-stranded polydeoxynucleotides that are chemically synthesized by known methods (such as phosphotriester, phosphite, or phosphoramidite chemistry, using solid phase techniques such as described in EP 266,032, or via deoxynucleoside H-phosphonate intermediates as described by Froehler et al. (1986), followed by purification, such as on polyacrylamide gels. In a specific embodiment, an oligonucleotide is a primer.

The term “primer,” as used herein, is meant to encompass any nucleic acid that is capable of priming the synthesis of a nascent nucleic acid in a template-dependent process.

The term “a short primer of random sequence” as used herein is defined as an oligonucleotide primer having the general formula dN₁-dN₂- . . . dNq, wherein dN represents a deoxyribonucleotide selected randomly from among dAMP, dCMP, dGMP, and dTMP and q represents integer 6 and above, preferably from 6 to 10.

The term “recombinant promoter” as used herein refers to a nucleic acid sequence which regulates expression of a particular nucleic acid sequence, wherein the promoter is genetically engineered through the application of recombinant DNA technology.

The term “template continuation (TC) oligonucleotide” as used herein is defined as an oligonucleotide used in a process of template-dependent synthesis of a complementary strand of DNA by a DNA polymerase using two templates in consecutive order and which are not covalently linked to each other by phosphodiester bonds. The synthesized cDNA strand is a single continuous strand complementary to both templates. In a specific embodiment of the present invention, the first template is poly (A)+ RNA and the second template is a template continuation oligonucleotide which preferably comprises at least two riboguanines at its 3′ end. It has a general formula dN₁-dN₂- . . . dNq-rN₁₋₇, where dN represents a deoxyribonucleotide selected from among dAMP, dCMP, dGMP, and dTMP and q represents integer 6 and above, preferably from 6 to 70, and rN represents a ribonucleotide, preferably riboguanine nucleotide. It typically provides a template for continuous synthesis of the first strand cDNA by attaching at the 3′ terminus of first strand cDNA through its sequence complementary to the 3′ terminal sequence of the first strand cDNA.

The term “terminal continuation reaction” as used herein is defined as a process of synthesizing the first strand cDNA using two templates. The first strand cDNA synthesis continues using a terminal continuation oligonucleotide as the second template at the termination of the first template. The synthesized cDNA is a single strand continuous molecule complementary to both first and second templates. In a specific embodiment of the present invention, the first template is RNA and the second template is a terminal continuation oligonucleotide which preferably comprises at least one riboguanine at the 3′ end. In some embodiments, at least two riboguanines are present at the 3′ end.

II. The Present Invention

A. General Embodiments

The present invention relates to a method of adding a nucleic acid sequence complementary to a “terminal continuation oligonucleotide”, to the 3′ end of a synthesized nucleic acid strand that is complementary to a target nucleic acid strand. The method comprises incubating the target nucleic acid strand in the presence of a terminal continuation oligonucleotide and a primer, the “first strand synthesis primer”, which is complementary to a sequence at the 3′ end, or upstream of the 3′ end, of the target nucleic acid strand. The first strand synthesis primer anneals or hybridizes to its complementary sequence on the target nucleic acid strand, which allows a polymerase to begin the synthesis of a nucleic acid strand complementary to the target nucleic acid strand. The polymerase also facilitates incorporation of sequence complementary to the terminal continuation oligonucleotide into the 3′ end of the synthesized nucleic acid strand by using the terminal continuation oligonucleotide as a template.

When using the above method to generate cDNA, the target nucleic acid strand is preferably RNA, more preferably mRNA. If mRNA is the target nucleic acid strand, then the first strand synthesis primer may preferably contain poly(dT). Random primers, for example random hexamers, and specifically designed primers may also be used as the first strand synthesis primer. With the addition of the first strand synthesis primer, terminal continuation oligonucleotide and reverse-transcriptase, a first-strand cDNA is synthesized that is complementary to the sequence of the target RNA strand sequence. In addition, the synthesized first strand cDNA contains the complementary sequence of the terminal continuation oligonucleotide at its 3′ end and the sequence of the first strand synthesis primer at its 5′ end.

The present invention provides a highly efficient method for the synthesis of second strand cDNA by being able to provide a sequence-specific priming method. As the complementary sequence of the terminal continuation oligonucleotide is incorporated into the 3′ end of first strand cDNA, second strand cDNA synthesis may be primed by the terminal continuation oligonucleotide. This obviates the need for inefficient second strand polymerases, such as Klenow and DNA Pol I, because the second strand synthesis is initiated by a primer, and not for example, by a hairpin loop. Therefore, the present invention provides for the use of robust polymerases, for highly efficient second strand cDNA synthesis.

Any polymerase may be used in the present invention, including but not limited to, polymerases from the following six families of polymerases: Pol I, Pol alpha, Pol beta, DNA-dependent RNA polymerases, reverse transcriptases, and RNA-dependent RNA polymerases (U.S. Pat. No. 5,614,365). Representative examples of Pol I-type DNA polymerases are: bacteriophage T7, T3, T4, T5, Spol, Spo2 and SP6 DNA polymerases, E. coli DNA polymerase I, Klenow fragment of E. coli DNA polymerase I, Thermus aquaticus DNA polymerase I (Taq), Bacillits stearothermophilus DNA polymerase (Bst), Thermus thermophilus DNA polymerase (Tth), Pyrococcus furiosus DNA polymerase (Pfu), Thermococcus litoralis DNA polymerase (Vent), and Thermus flavus DNA polymerase I. In addition to Taq, Vent, Bst, Tth and Pfu, other thermostable DNA polymerases are also included in the present invention. Examples of Pol-alpha or Polymerase II-type polymerases include E. coli DNA polymerase II and S. cerevisiae DNA polymerase II. Representative examples of RNA polymerases include: bacteriophage T7, T3 and SP6 RNA polymerases, E. coli RNA polymerase holoenzyme, E. coli RNA polymerase core enzyme, and human RNA polymerase I, II, III, and human mitochondrial RNA polymerase.

The present invention further provides the incorporation of cis-regulatory elements into synthesized nucleic acid strands through the use of the terminal continuation method. Cis-regulatory elements that may be introduced into nucleic acids, include but are not limited to, transcriptional promoters, bacteriophage transcriptional promoters, enhancers, silencers, methylation sites, origins of replication, matrix attachment regions, locus control regions and recombination signal sequences. Other similar elements known in the art may also be used.

The present invention also provides the incorporation of nucleic acids into synthesized nucleic acid strands by terminal continuation, where the incorporated nucleic acids may encode amino acids, stretches of amino acids and antigenic epitopes. The present invention fuirther provides the incorporation of nucleic acids into synthesized nucleic acid strands by terminal continuation, where the incorporated nucleic acids may serve to function as modification signals.

In one embodiment of the invention, the terminal continuation oligonucleotide and/or the first strand synthesis primer are designed to contain a transcriptional promoter, preferably a bacteriophage transcriptional promoter. In this embodiment of first strand cDNA synthesis, the cDNA strand may contain a transcriptional promoter at its 5′ end due to the annealing of a first strand synthesis primer that has a complementary sequence to the 3′ region of RNA in addition to the sequence that comprises the transcriptional promoter. The first strand cDNA may also contain a sequence complementary to a transcriptional promoter at its 3′ end if a terminal continuation oligonucleotide is designed to contain a transcriptional promoter. Alternatively, the first strand cDNA may contain a sequence of a transcriptional promoter at its 3′ end if a terminal continuation oligonucleotide is designed to contain the complementary sequence of a transcriptional promoter. A second strand of cDNA complementary to the first synthesized strand of cDNA may be synthesized using the first strand of cDNA as a template, and the terminal continuation oligonucleotide as a primer. Sense and/or antisense RNA amplification reaction may be subsequently performed by in vitro RNA transcription, as both the first strand and second strand of cDNA may contain transcriptional promoters incorporated at either the 5′ end, 3′end or both ends.

Using this methodology, even a small amount of starting RNA amplified linearly, such as RNA from a single cell, can be used for many downstream applications. Following the conversion of amplified RNA to double stranded cDNA, the down stream applications of amplified RNA include, but are not restricted to, probe generation, gene expression profiling, genetic polymorphism amplification and/or detection, cDNA microarray analysis, cDNA library construction, expression library construction, single cell cDNA library construction, subtraction library construction and competitive array hybridization. The synthesized sense RNA of a total starting RNA population can also be used as a template for in vitro protein translations, where the resultant protein may then be used for further downstream applications. A variety of reagent kits for the procedures may be developed as a result of, and are encompassed in, the present invention.

Any source of nucleic acid can be used as starting material, including but not limited to, DNA, RNA, ribosomal RNA, mitochondrial DNA, mitochondrial RNA, synthetic DNA, and synthetic RNA. Preferably, total RNA or poly (A)+ mRNA is used as starting material. A small amount (as low as picograms) of total RNA or mRNA extracted from single cells is sufficient for subsequent amplification. Sources of RNAs can include synthetic sources or biological sources, such as tissues from in vitro and in vivo preparations, including, but not restricted to, biopsy samples and post mortem tissues from a variety of species ranging from invertebrates to mammals including humans and genetically altered subjects. RNA from microbial genomes is also a source of starting genetic material. RNAs are extracted using standard molecular biological methods. Care must be taken to avoid RNase contamination along with inactivation of endogenous RNase activity.

Thus, the present invention concerns compositions and methods for amplification of RNA, preferably mRNA. The compositions and methods employ terminal continuation oligonucleotides described herein. The methods of the present invention comprise contacting RNA with a primer which can anneal to the RNA, a reverse transcriptase, and a terminal continuation oligonucleotide under conditions sufficient to permit the template-dependent extension of the annealed primer to generate an mRNA-cDNA hybrid, which is then followed by second strand cDNA synthesis.

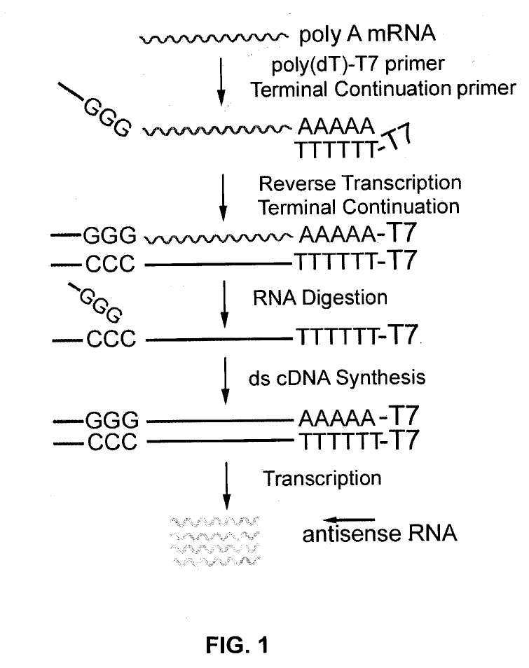

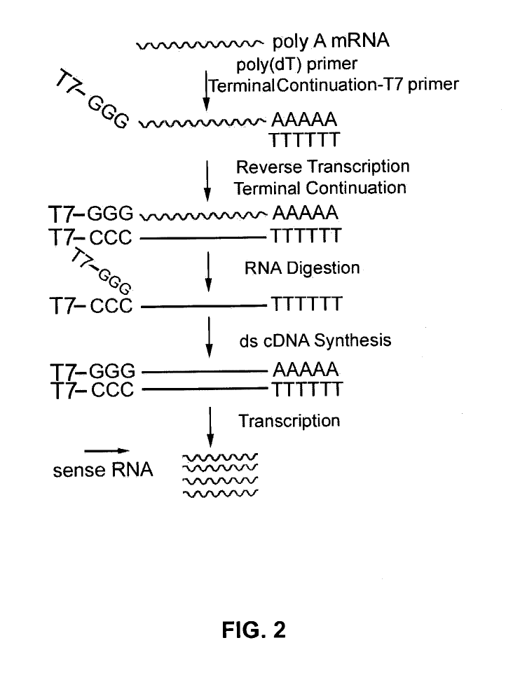

First strand synthesis is preferably primed with an oligonucleotide primer, the “first strand synthesis primer”, containing the sequence complement of a sequence at the 3′ end of the target nucleic acid. First strand synthesis may also be primed with an oligonucleotide primer containing the sequence complement of a sequence located upstream of the 3′ end of the target nucleic acid. If the target nucleic acid is RNA, examples of first strand synthesis primers include, but are not limited to, polythymidylate [poly(dT)s] or random sequences, such as random hexamer. In addition, the first strand synthesis primer can also include other desirable sequences, such as for example, a transcription promoter sequence, or a designed restriction enzyme digestion sequence (FIGS. 1 and 2).

It is preferred that a second primer, the “terminal continuation oligonucleotide”, is also present in the first strand synthesis reaction mixture. In addition, a sequence of a desired bacteriophage promoter, such as T7, T3, or SP6 or other functional sequences may optionally be a component sequence of the terminal continuation oligonucleotide (FIGS. 1 and 2).

It is preferred that the “terminal continuation oligonucleotide” contains at least one guanine or deoxyguanine (G or dG), or cytosine or deoxycytosine (C or dC) at its 3′ end, most preferably at least two G or dG or C or dC at its 3′ end. The terminal continuation oligonucleotide may alternatively contain at least one adenosine or deoxyadenosine (A or dA), or thymidine or deoxythymidine (T or dT) at its 3′ end. The terminal continuation oligonucleotide may also consist of a random sequence or nucleotide. It is preferred that the total length of the terminal continuation oligonucleotide is between about 8-100 nucleotides, more preferably about 15-75 nucleotides, most preferably about 20-50 nucleotides.

One reason for the preference that the “terminal continuation oligonucleotide” contains a short stretch of at least one guanine or deoxyguanine (G or dG), or cytosine or deoxycytosine (C or dC) at its 3′ end, is due to the efficiency in terminal continuation function. Both of the aforementioned structures have comparable efficiency in terminal continuation function. A complete or partial replacement of G, dG, C, dC at the 3′ end of a terminal continuation oligonucleotide with A, dA, T, dT decreases the efficiency of a terminal continuation reaction slightly. However, this reaction also produces terminal continuation products. The number of nucleotides and the sequence at the 3′ end of the terminal continuation oligonucleotide may be optimized empirically, and can readily be determined by the skilled artisan.

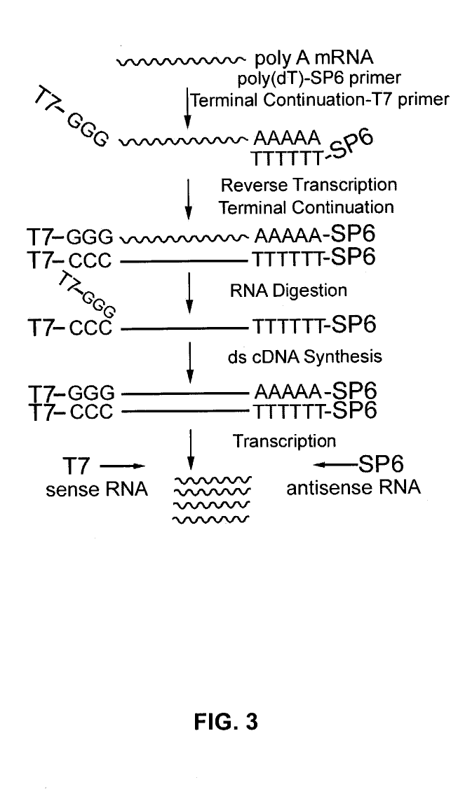

It is desirable for the method to match a primer with the appropriate promoter. For example, the same RNA transcription promoter is preferably not added to both the 5′ and 3′ termini of cDNA. However, two different promoters, such as T7 and T3, may be added at both the 5′ and 3′ ends of cDNA and direct either “sense” or “anti-sense” RNA synthesis. (FIG. 3). It is within the scope of the invention, that any promoter capable of initiating transcription can be used.

The second strand cDNA synthesis is preferably primed by an oligo(dNTP) with the sequence complementary to at least a portion of the terminal continuation oligonucleotide. In the embodiment where the synthesized cDNA strands contain transcriptional promoters, RNA may be transcribed with an RNA polymerase corresponding to the promoter. For example, T7 RNA polymerase may be used to transcribe RNA driven by a T7 promoter, whereas SP6 RNA polymerase may be used to transcribe RNA driven by a SP6 promoter. When two different promoters are attached at both ends of the cDNA, the RNA polymerase is chosen according to the “sense” or “antisense” orientation of the transcribed RNA desired.

More than one round of RNA amplification may be performed when necessary. During subsequent amplifications, the total population of RNA is reverse transcribed back into cDNA. The reverse transcription is primed either with specific primers attached to cDNA previously, by random primers, or by primers designed to amplify specific internal regions. In this embodiment of the invention, it is preferred that at least one RNA transcription promoter is incorporated into the subsequently synthesized double stranded cDNA.

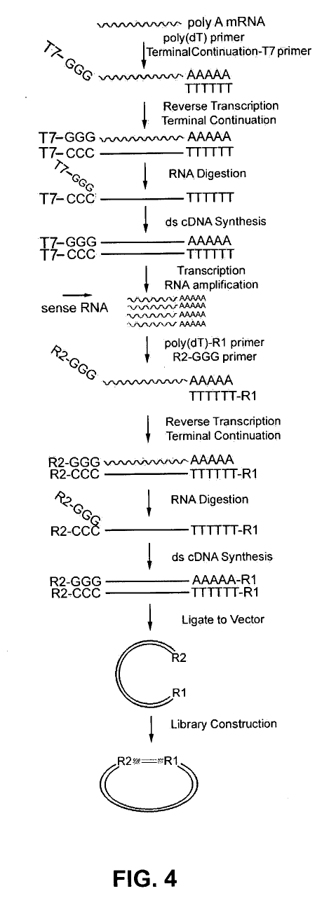

The cDNAs can be further engineered or altered by appropriate enzymatic manipulations prior to downstream applications. The downstream uses of the nucleic acid produced by the present method may include, for example, probe generation, gene expression profiling, genetic polymorphism profiling, cDNA library construction (FIG. 4), expression library construction, subtraction library construction, competitive array hybridization, in vitro translation, and clinical diagnostics independently or in combination with morphological examination.

The present invention may be conveniently developed into appropriate reagent kits for research or diagnostic purposes.

Thus, in a specific embodiment, the process of the present invention comprises at least the following steps:

1. Incubating a sample of poly(A)+RNA or total RNA with a poly (dT) primer or a short primer of random sequence which can anneal to mRNA and an enzyme that possesses reverse transcriptase activity under conditions sufficient to permit the template-dependent extension of the primer to generate an mRNA-cDNA hybrid. In some embodiments, the poly (dT) primer also comprises a bacteriophage promoter sequence, such as T7 RNA polymerase, T3 RNA polymerase, or SP6 RNA polymerase. In some embodiments, a small amount of total RNA or mRNA extracted from single cells is sufficient for subsequent amplification.

2. Incubating the first-strand cDNA synthesis mixture obtained from step 1 with a terminal continuation oligonucleotide of the present invention. The terminal continuation oligonucleotide has at least one riboguanine residue at its 3′-end, a nucleotide sequence at its 5′-end which may be variable, and in some embodiments a restriction enzyme digestion site, an RNA synthesis promoter, a protein translation start signal, or a combination thereof.

3. Second strand cDNA synthesis.

4. In vitro transcription.

Using the methods of the present invention with conventional procedures, first-strand cDNA synthesis is carried out using RNA as a template for reverse transcription. A primer is annealed to RNA forming a primer:RNA complex. Extension of the primer is catalyzed by reverse transcriptase, or by a DNA polymerase possessing reverse transcriptase activity, in the presence of adequate amounts of other components necessary to perform the reaction, for example, deoxyribonucleoside triphosphates dATP, dCTP, dGTP and dTTP, Mg²⁺, and optimal buffer. A variety of reverse transcriptases can be used. Preferably, the reverse transcriptase is isolated from Moloney murine leukemia virus (M-MLV) (U.S. Pat. No. 4,943,531) or M-MLV reverse transcriptase lacking RNaseH activity (U.S. Pat. No. 5,405,776), avian myeloblastosis virus (AMV), human T-cell leukemia virus type I (HTLV-I), Rous-associated virus 2 (RAV2), bovine leukemia virus (BLV), Rous sarcoma virus (RSV), human immunodeficiency virus (HIV) or Thermus aquaticus (Taq) or Thermus thermophilus (Tth) (U.S. Pat. No. 5,322,770). These reverse transcriptases may be isolated from an organism itself or, in some cases, obtained commercially. Reverse transcriptases useful with the subject invention can also be obtained from cells expressing cloned genes encoding the enzyme. As a starting material for cDNA synthesis, poly(A)+RNA or total RNA from yeast and higher organisms such as plants or animals can be used. The first-strand cDNA synthesis step of the subject method can include terminal continuation oligonucleotides of the present invention in the reaction mixture, but are not a necessary component for carrying out first-strand cDNA synthesis. Thus, it is understood that terminal continuation oligonucleotide molecules can be included in the first-strand reaction composition (for example, during the first primer annealing to RNA or when contacting the RNA with an enzyme possessing reverse transcriptase activity) or the oligonucleotides can be added in the course of, or after completion of, the first-strand cDNA synthesis reaction.

In an alternative embodiment, in lieu of a poly (dT) primer, a primer to an inner, non-poly(A)+ portion of the mRNA is utilized. These oligonucleotide primer(s) have the general formula dN₁-dN2 - . . . dNq, where dN represents a deoxyribonucleotide selected from among dAMP, dCMP, dGMP, and dTMP and q represents integer 6 and above.

In an alternative embodiment, a population of short primers of random sequences can be used. The primers are sufficiently short, preferably 6-10 deoxyoligo nucleic acids, and the sequences are sufficiently variable that every RNA present has at least one primer that has the sequence complementary to it and anneals to it to prime the synthesis of a first strand cDNA.

Following the complete synthesis of the first strand cDNA, the terminal transferase activity of reverse transcriptase adds a few additional nucleotides, primarily deoxycytidine and/or deoxyguanine, to the 3′ end of the newly synthesized cDNA strand independent of template. The terminal continuation oligonucleotide, which in some embodiments has an oligo (rG) sequence at its 3′ end, base pairs with the deoxycytidine-rich stretch of nucleotides present on the first cDNA strand, creating an extended template. Reverse transcriptase then continues synthesis of cDNA complementary to the terminal continuation oligonucleotide attached to the terminal of the first stranded cDNA. Thus, the full extension product of the first cDNA synthesis comprises both sequences complementary to the RNA and to the terminal continuation oligonucleotide.

Replacement of the RNA portion of the mRNA:cDNA hybrid with a second-strand cDNA entails removal of the RNA strand in RNA:DNA molecules, and also include DNA synthesis by a DNA polymerase. In a specific embodiment, RNAse H is utilized. In an alternative embodiment, heating in the presence of appropriate concentration (such as in a range of 0.001 mM to 0.15 mM) of magnesium chloride. DNA synthesis is continuous and no ligation step is necessary.

The second strand cDNA synthesis is primed by an oligo (dNTP) with the sequence identical to whole or a portion of the terminal continuation oligonucleotide. A variety of DNA polymerases can be used, such as E. coli DNA polymerase I, bacteriophage T4 DNA polymerase, bacteriophage T7 DNA polymerase, and large fragment of E. coli DNA polymerase I (Klenow fragment). In a specific embodiment, a thermostable and robust DNA polymerase, Taq DNA polymerase, is used for second strand cDNA synthesis.

In other embodiments of the present invention, the present invention is directed to amplification and detection of RNA from a histologically-stained tissue. Until now, the amplification of RNA by in vitro transcription from the same presently histologically-stained source of tissue has not been known, although methods to amplify genetic signals by PCR based methods are known. That is, it is known to use PCR methods, which are exponential, to amplify a dsDNA molecule or to amplify an mRNA by RT-PCR, but the amplification of an RNA molecule derived from the dsDNA molecule, particularly in a linear fashion, is unknown. In a preferred embodiment, the RNA is amplified by aRNA methods (Van Gelder et al. (1990); Eberwine et al. (1992); U.S. Pat. No. 5,545,522), all of which are incorporated herein by reference in their entirety) or by other in vitro transcription methods, such as are the subject of the present invention.

In an object of the present invention, the amplified RNA population is used as a clinical diagnostic tool independently or in combination with morphological examination, such as regarding the treatment and/or diagnosis of an individual.

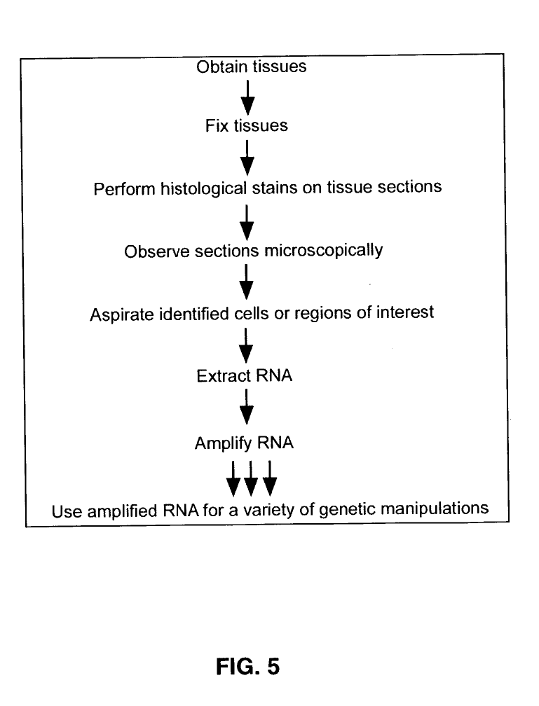

The present invention describes a method for amplification of RNA populations from histologically stained tissues and cells through in vitro transcription (FIG. 5). The amplified RNAs could be further genetically manipulated for the applications of down stream investigations, including, but not restricted to, RNA amplification, cDNA microarray analysis, subtractive hybridization, RT-PCR, library constructions, and clinical molecular diagnoses.

1) Biological tissues from in vitro and in vivo preparations can be used, including, but not restricted to, biopsy samples and post mortem tissues from a variety of species ranging from invertebrates to mammals, including genetically altered subjects and humans.

2) The sample for the present invention is directed to any cellular material including but not limited to muscle, connective tissue, skin, brain, liver, urine, bone marrow, touch preps of surgical specimens, fine needle aspirates and all cellular body fluids, including cerebrospinal fluid, blood, mucus, saliva, nipple aspirates, urine, sweat, and feces. In addition samples can include any pathological tissue including but not limited to tumors, lymph nodes, lesions, blood vessels, and traumatic injured tissues.

3) The fixation conditions are flexible, as both fresh tissues and fixed tissues can be utilized. The samples can be fixed by a wide variety of reagents, including but not restricted to, acetone, aldehyde derivatives, ethanol, and combinations therein. The critical step for the fixation is use of RNAse-free conditions and buffers, prompt accession of tissues, and low temperature. RNAs are preserved best under these conditions. Frozen tissues and various cross-linking and precipitating fixatives such as formalin, paraformaldehyde, acetone and ethanol are utilized. A skilled artisan recognizes that the present invention is utilized to amplify RNA from cells/tissues as well as body fluids, (e.g., cerebrospinal fluid, blood, saliva, urine, feces, sweat).

4) After fixation, tissues are sectioned and histological stains applied for cellular visualization and diagnostic prediction prior to the extraction of RNA. The histological stains include all preparations that depict cellular, regional, laminar, and nuclear structures within tissue samples. Examples of histological stains that can be utilized by this invention include: hematoxylin and eosin, thionin, cresyl violet, acridine orange, and reduced silver preparations. When the presence of RNA is in doubt, acridine orange staining can be used to visualize RNA (Ginsberg et al., 1997; Ginsberg et al., 1998) in the tissues and cell(s) of interest before RNA extraction and subsequent amplification.

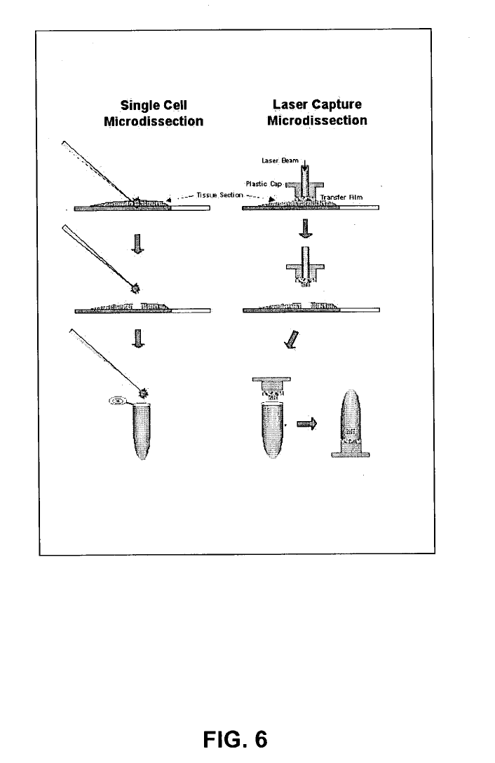

5) Individually-identified cells or populations of cells are dissected from tissues using a micropipette attached to a micromanipulator or by laser capture microdissection (FIG. 6).

6) Microdissected cells should be immediately merged into chaotropic cell lysis buffers to inactivate RNase activity instantaneously. Commercially available RNA extraction reagents (such as trizol) can also be used. In general, no homogenization step is necessary. The usage of an inert carrier, such as glycogen or linear acrylamide, is helpful for maximum RNA precipitation.

7) RNAs of such minute amount will almost always have to be amplified first prior to desired down stream usage. The first step of the RNA amplification is to synthesize ds-cDNA templates. This first strand cDNA is synthesized with a reverse transcriptase primed by an oligonucleotide that anneals to RNAs. In some embodiments, a TC primer is included in the first strand cDNA synthesis mixture, which will serve as a template at the 3′ terminal of the synthesized first strand cDNA. The second strand cDNA is synthesized by a DNA polymerase using first strand cDNA as template and primed by a primer with the sequence substantially similar to TC primer.

8) RNA extracted from histologically stained tissues or cells is amplified through in vitro RNA amplification. In practice, in vitro RNA transcription needs a promoter to drive the reaction. The best promoter candidates are the bacteriophage promoters T7, T3, and SP6.

9) A transcription promoter can be annealed to the 3′ of first strand cDNA by priming mRNA with a specific poly(T) primer that contains the promoter sequence. Alternatively, a promoter can be attached to the 5′ of first strand cDNA through terminal continuation (U.S. Patent Application filed Feb. 14, 2001 entitled “RNA Amplification Method.”)

10) This procedure can be developed conveniently into reagent kits with the essential component of histological stains, an RNA extraction reagent, an RNA precipitation carrier, primers and enzymes for the synthesis of ds-cDNA template and enzymes in vitro RNA transcription.

11) Various modifications or changes in light thereof will be suggested to persons skilled in the art, and are to be included in the scope of the invention.

Also within the scope of the present invention is a method of hybridization using probes generated from an amplified RNA population. RNA probes generated according to the present invention will be labeled, either by radioisotopes, fluorescent dye, biotin and other reporter groups by conventional chemical or enzymatic labeling procedures. On the other hand, a complementary cDNA can be further synthesized and labeled using RNA generated in present invention as a template. Labeled RNA or cDNA can then be used in standard hybridization assays known in the art, i.e., the labeled RNA or cDNA is contacted with the defined oligonucleotide/polynucleotides corresponding to a particular set of the genes immobilized on a solid surface for a sufficient time to permit the formation of patterns of hybridization on the surfaces caused by hybridization between certain polynucleotide sequences in the hybridization probe with the certain immobilized defined oligonucleotide/polynucleotides. The hybridization patterns using available conventional techniques, such as scintillation counting, autoradiography, fluorescence detection, colorimetric assays, optical density assessments, or light emission measurement. Techniques and conditions for labeling, hybridization and detection are well known in the art (see, e.g. Sambrook et al., 1989; Ausubel et al., 1994).

In a preferred embodiment, a microarray is probed with RNA or cDNA generated by methods of the present invention. A microarray is usually a solid support, either a glass slide or a membrane, with hundreds or even thousands known genes or DNAs printed on it. As used herein, the term “solid support” refers to any known substrate which can be used for the immobilization of a binding ligand or oligonucleotide/polynucleotide sequences by any known method. A distinct pattern of hybridization will be generated by probing a microarray with RNA or cDNA generated with the present invention, which leads to the establishment of a gene expression profile of the tissue from which RNA is extracted.

In another embodiment, a RNA or cDNA generated with the present invention can be separated in an agarose gel, transferred to a solid support, such as a nylon or a nitrocellulose membrane, and probed with a labeled known RNA or DNA as in Northern or Southern hybridization analysis.

Also within the scope of the present invention is a method for generating libraries containing cDNAs generated from amplified RNAs. Conventional methods used to generate cDNA libraries require either large quantities starting materials or a PCR step to amplify small quantity of starting materials. Both methods are not suitable for the generation of cDNA from a homogeneous population of cells due to the difficulty of obtaining large quantities of pure material from a homogeneous population. Moreover, a low copy gene can rendered undetectable during PCR amplification. The present method provides an improved alternative to generate cDNA libraries from a homogeneous cell population.

In another aspect, the invention provides methods wherein the resulting cDNA product generated can be used as a starting material for use with cDNA subtraction methods. Specifically, the method of the subject invention can be used in conjunction with cDNA subtraction procedures to prepare a cDNA population containing highly enriched representation of cDNA species that are present in one DNA population (the tester population), but that are less abundant or absent in another DNA population (the driver population). Tester and driver ds cDNA amplified by the methods of the present invention can be used in combination with suppression subtractive hybridization technology described previously (see e.g. U.S. Pat. No. 5,565,340 and U.S. Pat. No. 5,436,142).

A person familiar with the art of the field will be able to devise modifications of the above method for the detection of genes present in the RNA population generated in the present invention.

Thus, the use of the terminal continuation method provides a substantially improved sensitivity and efficiency of linear RNA amplification. The benefit of the improvement is the detection of the presence and the quantity of multiple genes from minimum quantity of starting materials.

III. In vitro Transcription

In vitro transcription requires a purified, linear ds cDNA template, such as is generated with the methods of the present invention, containing a promoter, ribonucleotide triphosphates, a buffer system that preferably includes DTT and magnesium, and an appropriate bacteriophage RNA polymerase. A skilled artisan recognizes that the exact conditions used in the transcription reaction depend on the quantity and quality of RNA needed for a specific application (the reaction conditions will be different for generating labeled RNA as hybridization probes compared to those reaction conditions for obtaining large quantity of RNAs).

The common RNA polymerases used in in vitro transcription reactions are SP6, T7 and T3 polymerases, named for the bacteriophages from which they were cloned. The genes for these proteins have been overexpressed in Escherichia coli, and the polymerases have been purified and are commercially available. RNA polymerases are DNA template-dependant with distinct and very specific promoter sequence requirements. The promoter consensus sequences for each of the phage RNA polymerases are as follows, wherein the first base incorporated into the transcript is bolded, and the minimum sequence required for efficient transcription is underlined:

| | | |
|---|---|---|
| T7: | 5′-TAATACGACTCACTATAGGGAGA-3′ | (SEQ ID NO:1) |
| SP6: | 5′-ATTTAGGTGACACTATAGAAGNG-3′ | (SEQ ID NO:2) |
| T3: | 5′-AATTAACCCTCACTAAAGGGAGA-3′ | (SEQ ID NO:3) |

After the RNA polymerase binds to its double-stranded DNA promoter, the polymerase separates the two DNA strands and uses the 3′ to 5′ strand as template for the synthesis of a complementary 5′ to 3′ RNA strand. Depending on the orientation of DNA sequence relative to the promoter, as generated by the methods described herein, the template may be designed to produce sense strand or antisense strand RNA. Specifically, a transcription promoter has to be attached to a dsDNA template through the mechanism of terminal continuation when sense RNA is to be synthesized, whereas a transcription promoter has to be attached to a ds RNA template through annealing a poly(dT) primer containing a promoter sequence to an mRNA molecule when antisense RNA is to be synthesized. When designing a transcription template, it must be decided whether sense or antisense transcripts are needed. If the RNA is to be used as a probe for hybridization to messenger RNA (e.g. in situ hybridization, or nuclease protection assays), complementary antisense transcripts are required. In contrast, sense strand transcripts are used when performing expression, structural or functional studies or when constructing a standard curve for RNA quantitation using an artificial sense strand RNA. Either sense or antisense RNA can be used in microarray analysis or reverse northern hybridization.

By convention, the single strand of a DNA sequence shown in scientific journals and databases is the coding, (+), or “sense strand”, identical in sequence (with T's changed to U's) to its mRNA copy. The +1 G of the RNA polymerase promoter sequence in the DNA template is the first base incorporated into the transcription product (see above). To make sense RNA, the 5′ end of the coding strand must be adjacent to, or just downstream of, the +1 G of the promoter. For antisense RNA to be transcribed, the 5′ end of the noncoding strand must be adjacent to the +1 G.

IV. Nucleic Acid Detection

In some embodiments of the present invention, detection of nucleic acids, particularly those amplified by the methods described herein, is desired. In a preferred embodiment, a microarray is probed with RNA generated by methods of the present invention.

A. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective (Sambrook et al., 1989). Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the present invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probes and target sequences would be particularly suitable for isolating specific genes or for detecting specific mRNA transcripts. It is generally appreciated that conditions can be rendered more stringent by the addition of increasing amounts of formamide.

For certain applications, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt (such as NaCl), at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. In a specific embodiment, 50% formamide solutions with 6 XSSPE, KCl, MgCI2, 5×Denhardt's. 1M NaPPi, and 200 ng/ml sheared salmon sperm DNA are used.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCRTM, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

In the present invention, it is desirable to first convert an RNA to a complementary DNA, and in a specific embodiment, the resultant cDNA is amplified, such as with primers. Typically, primers are oligonucleotides from ten to twenty and/or thirty base pairs in length, but longer sequences can be employed. Primers may be provided in double-stranded and/or single-stranded form, although the single-stranded form is preferred.

The technique of “polymerase chain reaction,” or “PCR,” as used herein generally refers to a procedure wherein minute amounts of a specific piece of nucleic acid, RNA and/or DNA, are amplified as described in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987); Erlich, ed., PCR Technology, Stockton Press, N.Y., (1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid that is complementary to a particular nucleic acid.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara etal., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

V. Kits

All of the essential materials and/or reagents required for amplifying mRNA according to the methods of the present invention in a sample may be assembled together in a kit. This generally will comprise a probe or primers designed to hybridize specifically to individual nucleic acids of interest in the practice of the present invention. In specific embodiments, the terminal continuation primer, a short random primer, and/or a poly (dT) primer are included in the kit. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

VI. Primer Synthesis

In the present invention, oligonucleotide synthesis for primers necessary to practice methods of the present invention may be performed according to one or more of the standard methods described in the art. See, for example, Itakura and Riggs (1980). Additionally, U.S. Pat. No. 4,704,362; U.S. Pat. No. 5,221,619; and U.S. Pat. No. 5,583,013 each describe various methods of preparing synthetic structural genes.

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

A. Diester Method

The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

B. Triester Method

The main difference between the diester and triester methods is the presence in the latter of an extra protecting group on the phosphate atoms of the reactants and products (Itakara et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

C. Polynucleotide Phosphorylase Method

This is an enzymatic method of DNA synthesis that can be used to synthesize many useful oligodeoxynucleotides (Gillam et al., 1978; Gillam et al., 1979). Under controlled conditions, polynucleotide phosphorylase adds predominantly a single nucleotide to a short oligodeoxynucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

D. Solid-Phase Methods

Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic DNA synthesizers.

Phosphoramidite chemistry (Beaucage and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

VII. Cell Samples

The cell samples to be subjected to methods of the present invention are, in an object of the present invention, from an individual with an unknown or uncertain medical condition or whose medical condition is known but means of therapy remains to be determined. In an alternative embodiment, the cell samples are from individuals whose cells are being tested for inclusion in a database for genomics analysis.

The sample for the present invention is directed to any cellular material including but not limited to urine, bone marrow, blood, touch preps of surgical specimens, fine needle aspirates and all cellular body fluids, including cerebrospinal fluid, blood, mucus, saliva, urine sweat, and feces. In one specific embodiment, the cell is fixed, such as by fixatives known in the art, including acetone, aldehyde derivatives, ethanol, and combinations thereof. In an alternative embodiment, the cell is from fresh tissue. Regardless, it is preferable to maintain the cell sample in RNAse-free conditions and buffers wherein the RNA is preserved.

A skilled artisan recognizes that touch prep specimens are generated by smearing or pressing onto a slide, applying pressure to the tissue, and fixing in ethanol under cool temperatures. In a specific embodiment, the tissue is extracted surgically and smeared onto a glass slide by applying relatively weak pressure to tumor tissue and relatively strong pressure to normal tissue, followed by fixing in about 100% ethanol for approximately 10 minutes at about 4° C. In another specific embodiment, the samples to be analyzed by methods of the present invention are originally frozen in liquid nitrogen and stored at about −80° C.

In a specific embodiment, the sample to be analyzed contains primarily a cancer cell, an epithelial cell, a bone marrow cell, a red blood cell, a white blood cell, a muscle cell, a bone cell, a connective tissue cell, a nerve cell and/or a brain cell.

Specimens, or samples, of a cellular body fluid or material are received and may be concentrated and/or diluted, depending on the source. In a specific embodiment, the samples are further processed or prepared. For example, cell suspensions may be purified by standard techniques including ficoll-hypaque density centrifugation. Microscopic slides are prepared using the concentrated or processed specimen to optimize cellular content and, in a preferred embodiment, are stained with propidium iodide for DNA content and with stains or markers for additional cell characteristics such as cytokeratin, CD19, CD34, CD3, annexin V, and a combination thereof.

VIII. Histological Staining

In particular embodiments of the present invention, the tissue or cell from which the RNA is amplified is histologically stained at some point prior to the genetic signal analysis. The histological stains include all preparations that depict cellular, regional, and laminar structures within tissue samples. The histological stains also include all preparations that depict nuclear, cytoplasmic, mitochondria, centrioles, rough endoplasmic reticulum, smooth endoplasmic reticulum, Golgi apparatus structures, peroxisomes, endosomes, lysosomes and carbohydrates, glycoproteins, lipids and nucleoproteins components. The examples of staining methods include hematoxylin and eosin, Congo red, Gallyas silver, thioflavin, Masson's trichrome, Movat's pentachrome, Verhoeff-van Gieson, Ricinus communis lectin, phosphorungstic acid hematoxylin, Prussian blue, Oil red O, Sudan, Fontana-Masson, bleached granules, Giemsa, Mucicarmine, alcian blue-PAS, Luxol fast blue, toluidine blue, Holmes, Hicks, methyl green-pyronine, thionin, cresyl violet, acridine orange, and reduced silver preparations as opposed to protein mediated, e.g. immunohistochemistry, or nucleic acid mediated, e.g. in situ hybridization or in situ PCR mediated staining.

A skilled artisan recognizes that there are a variety of histological stains known in the art, examples of which are listed in Table 1.

TABLE 1
COMMONLY USED HISTOLOGICAL STAINS

| Name | Class | Common name |
|---|---|---|
| Acid black 1 | Disazo | Amido black 10B |
| Acid blue 22 | Triarylmethane | Water blue I |
| Acid blue 93 | Triarylmethane | Methyl blue |
| Acid fuchsin | Triarylmethane | Acid fuchsin |
| Acid green | Triarylmethane | Light green SF yellowish |
| Acid green 1 | Nitroso | Naphthol green B |
| Acid green 5 | Triarylmethane | Light green SF yellowish |
| Acid magenta | Triarylmethane | Acid fuchsin |
| Acid orange 10 | Monoazo | Orange G |
| Acid red 26 | Monoazo | Xylidine ponceau |
| Acid red 29 | Azo | Chromotrope 2R |
| Acid red 44 | Azo | Ponceau 6R |
| Acid red 51 | Fluorone | Erythrosin B |
| Acid red 66 | Disazo | Biebrich scarlet |
| Acid red 87 | Fluorone | Eosin Y ws |
| Acid red 91 | Fluorone | Eosin B |
| Acid red 92 | Fluorone | Phloxine B |
| Acid red 94 | Fluorone | Rose bengal |
| Acid red 101 | Quinone-Imine | Azocarmine B |
| Acid red 103 | Quinone-Imine | Azocarmine B |
| Acid roseine | Triarylmethane | Acid fuchsin |
| Acid rubin | Triarylmethane | Acid fuchsin |
| Acid violet 19 | Triarylmethane | Acid fuchsin |
| Acid yellow 1 | Nitro | Naphthol yellow S |
| Acid yellow 9 | Nitro | Fast yellow |
| Acid yellow 23 | Azo | Tartrazine |
| Acid yellow 24 | Nitro | Martius yellow |
| Acid yellow 36 | Azo | Metanil yellow |
| Acid yellow 73 | Fluorone | Fluorescein |
| Acid yellow S | Nitro | Naphthol yellow S |
| Acridine orange | Acridine | Acridine orange |
| Acriflavine | Acridine | Acriflavine |
| Alcian blue | Phthalocyanine | Alcian blue |
| Alcian yellow | Azo | Alcian yellow |
| Alcohol soluble eosin | Fluorone | Ethyl eosin |
| Alizarin | Anthraquinone | Alizarin |
| Alizarin blue 2RC | Anthraquinone | Anthracene blue SWR |
| Alizarin carmine | Anthraquinone | Alizarin red S |
| Alizarin cyanin BBS | Anthraquinone | Alizarin cyanin BBS |
| Alizarol cyanin R | Triarylmethane | Eriochrome cyanin R |
| Alizarin red S | Anthraquinone | Alizarin red S |
| Alizarin purpurin | Anthraquinone | Purpurin |
| Aluminon | Triphenylmethane | Chrome violet CG |
| Amido black 10B | Disazo | Amido black 10B |
| Amidoschwarz | Disazo | Amido black 10B |
| Aniline blue WS | Triarylmethane | Aniline blue WS |
| Anthracene blue SWR | Anthraquinone | Anthracene blue SWR |
| Auramine O | Triarylmethane | Auramine O |
| Azocarmine B | Quinone-Imine | Azocarmine B |
| Azocarmine G | Quinone-Imine | Azocarmine B |
| Azoic diazo 5 | Diazonium salt | Fast red B |
| Azoic diazo 48 | Diazonium salt | Fast blue B |
| Azure A | Thiazin | Azure A |
| Azure B | Thiazin | Azure B |
| Azure C | Thiazin | Azure C |
| Basic blue 8 | Triarylmethane | Victoria blue 4R |
| Basic blue 9 | Thiazin | Methylene blue |
| Basic blue 12 | Oxazin | Nile blue A |
| Basic blue 15 | Triarylmethane | Night blue |
| Basic blue 17 | Thiazin | Toluidine blue O |
| Basic blue 20 | Triarylmethane | Methyl green |
| Basic blue 26 | Triarylmethane | Victoria blue B |
| Basic brown 1 | Disazo | Bismarck brown Y |
| Basic fuchsin | Triarylmethane | Basic fuchsin |
| Basic green 4 | Triarylmethane | Malachite green |
| Basic orange 14 | Acridine | Acridine orange |
| Basic red 2 | Safranin | Safranin O |
| Basic red 5 | Eurhodin | Neutral red |
| Basic red 9 | Triarylmethane | Pararosanilin |
| Basic violet 2 | Triarylmethane | New fuchsin |
| Basic violet 3 | Triarylmethane | Crystal violet |
| Basic violet 4 | Triarylmethane | Ethyl violet |
| Basic violet 10 | Rhodamine | Rhodamine B |
| Basic violet 14 | Triarylmethane | Rosanilin |
| Basic yellow 1 | Thiazole | Thioflavine T |
| Basic yellow 2 | Diarylmethane | Auramine O |
| Biebrich scarlet | Disazo | Biebrich scarlet |
| Bismarck brown Y | Disazo | Bismarck brown Y |
| Brilliant crystal scarlet 6R | Azo | Ponceau 6R |
| Calcium red | Anthraquinone | Nuclear fast red |
| Carmine | Natural | Carmine |
| Carminic acid | Natural | Carmine |
| Celestine blue B | Oxazin | Celestine blue B |
| China blue | | Aniline blue |
| Cochineal | Natural | Carmine |
| Coelestine blue | Oxazin | Celestine blue B |
| Chrome violet CG | Triphenylmethane | Chrome violet |
| Chromotrope 2R | Azo | Chromotrope 2R |
| Chromoxane cyanin R | Triarylmethane | Eriochrome cyanin R |
| Congo corinth | Disazo | Congo corinth |
| Congo red | Disazo | Congo red |
| Cotton blue | Triarylmethane | Methyl blue |
| Cotton red | Disazo | Congo red |
| Croceine scarlet | Diazo | Biebrich scarlet |
| Crocin | Natural | Saffron |
| Crystal ponceau 6R | Azo | Ponceau 6R |
| Crystal violet | Triarylmethane | Crystal violet |
| Dahlia | Triarylmethane | Hoffman's violet |
| Diamond green B | Triarylmethane | Malachite green |
| Direct blue 14 | Daizo | Trypan blue |
| Direct blue 58 | Disazo | Evans blue |
| Direct red | Disazo | Congo red |
| Direct red 10 | Disazo | Congo corinth |
| Direct red 28 | Disazo | Congo red |
| Direct red 80 | Tetrakisazo | Sirius red F3B |
| Direct yellow 7 | Thiazole | Thioflavine S |
| Eosin B | Fluorone | Eosin B |
| Eosin Bluish | Fluorone | Eosin B |
| Eosin | Fluorone | Eosin Y ws |
| Eosin Y | Fluorone | Eosin Y ws |
| Eosin yellowish | Fluorone | Eosin Y ws |
| Eosinol | Fluorone | Eosinol |
| Erie garnet B | Disazo | Congo corinth |
| Eriochrome cyanin R | Triarylmethane | Eriochrome cyanin R |
| Erythrosin B | Fluorone | Erythrosin B |
| Ethyl eosin | Fluorone | Ethyl eosin |
| Ethyl green | Triarylmethane | Ethyl green |
| Ethyl violet | Triarylmethane | Ethyl violet |
| Evans blue | Disazo | Evans blue |
| Fast blue B | Diazonium salt | Fast blue B |
| Fast green FCF | Triarylmethane | Fast green FCF |
| Fast red B | Diazonium salt | Fast red B |
| Fast yellow | Nitro | Fast yellow |
| Fluorescein | Fluorone | Fluorescein |
| Food green 3 | Triarylmethane | Fast green FCF |
| Gallein | Fluorone | Gallein |
| Gallamine blue | Oxazin | Gallamine blue |
| Gallocyanin | Oxazin | Gallocyanin |
| Gentian violet | Triarylmethane | Methyl violet 2B |
| Haematein | Natural | Hematein |
| Haematine | Natural | Hematein |
| Haematoxylin | Natural | Hematoxylin |
| Helio fast rubin BBL | Anthraquinone | Nuclear fast red |
| Helvetia blue | Triarylmethane | Methyl blue |
| Hematein | Natural | Hematein |
| Hematine | Natural | Hematein |
| Hematoxylin | Natural | Hematoxylin |
| Hoffman's violet | Triarylmethane | Hoffman's violet |
| Imperial red | Fluorone | Eosin B |
| Ingrain blue | Phthalocyanine | Alcian blue |
| Ingrain blue 1 | Phthalocyanine | Alcian blue |
| Ingrain yellow 1 | Azo | Alcian yellow |
| INT | Tetrazolium salt | Iodonitrotetrazolium |
| Kermes | Natural | Kermes |
| Kermesic acid | Natural | Kermes |
| Kernechtrot | Anthraquinone | Nuclear fast red |
| Lac | Natural | Laccaic acid |
| Laccaic acid | Natural | Laccaic acid |
| Lauth's violet | Thiazin | Thionin |
| Light green | Triarylmethane | Light green SF yellowish |
| Lissamine green SF | Triarylmethane | Light green SF yellowish |
| Luxol fast blue | Phthalocyanine | Luxol fast blue MBS |
| Magenta O | Triarylmethane | Pararosanilin |
| Magenta I | Triarylmethane | Rosanilin |
| Magenta II | Triarylmethane | Magenta II |
| Magenta III | Triarylmethane | New fuchsin |
| Malachite green | Triarylmethane | Malachite green |
| Manchester brown | Disazo | Bismarck brown Y |
| Martius yellow | Nitro | Martius yellow |
| Merbromin | Fluorone | Mercurochrome 220 |
| Mercurochrome | Fluorone | Mercurochrome 220 |
| Metanil yellow | Azo | Metanil yellow |
| Methylene azure A | Thiazin | Azure A |
| Methylene azure B | Thiazin | Azure B |
| Methylene azure C | Thiazin | Azure C |
| Methylene blue | Thiazin | Methylene blue |
| Methyl blue | Triarylmethane | Methyl blue |
| Methyl green | Triarylmethane | Methyl green |
| Methyl violet | Triarylmethane | Methyl violet 2B |
| Methyl violet 2B | Triarylmethane | Methyl violet 2B |
| Methyl violet 10B | Triarylmethane | Crystal violet |
| Mordant blue 3 | Triarylmethane | Eriochrome cyanin R |
| Mordant blue 10 | Oxazin | Gallocyanin |
| Mordant blue 14 | Oxazin | Celestine blue B |
| Mordant blue 23 | Anthraquinone | Alizarin cyanin BBS |
| Mordant blue 32 | Anthraquinone | Anthracene blue SWR |
| Mordant blue 45 | Oxazin | Gallamine blue |
| Mordant red 3 | Anthraquinone | Alizarin red S |
| Mordant red 11 | Anthraquinone | Alizarin |
| Mordant violet 25 | Fluorone | Gallein |
| Mordant violet 39 | Triphenylmethane | Chrome violet CG |
| Naphthol blue black | Disazo | Amido black 10B |
| Naphthol green B | Nitroso | Naphthol green B |
| Naphthol yellow S | Nitro | Naphthol yellow S |
| Natural black 1 | Natural | Hematein |
| Natural red | Anthraquinone | Purpurin |
| Natural red 3 | Natural | Kermes |
| Natural red 4 | Natural | Carmine |
| Natural red 8 | Anthraquinone | Purpurin |
| Natural red 16 | Anthraquinone | Purpurin |
| Natural red 25 | Natural | Laccaic acid |
| Natural red 28 | Natural | Orcein |
| Natural yellow 6 | Natural | Saffron |
| NBT | Tetrazolium salt | Nitro blue tetrazolium |
| Neutral red | Eurhodin | Neutral red |
| New fuchsin | Triarylmethane | New fuchsin |
| Niagara blue 3B | Diazo | Trypan blue |
| Night blue | Triarylmethane | Night blue |
| Nile blue | Oxazin | Nile blue A |
| Nile blue A | Oxazin | Nile blue A |
| Nile blue oxazone | Oxazone | Nile red |
| Nile blue sulphate | Oxazin | Nile blue A |
| Nile red | Oxazone | Nile red |
| Nitro BT | Tetrazolium salt | Nitro blue tetrazolium |
| Nitro blue tetrazolium | Tetrazolium salt | Nitro blue tetrazolium |
| Nuclear fast red | Anthraquinone | Nuclear fast red |
| Oil red O | Disazo | Oil red O |
| Orange G | Monoazo | Orange G |
| Orcein | Natural | Orcein |
| Pararosanilin | Triarylmethane | Pararosanilin |
| Phloxine B | Fluorone | Phloxine B |
| Picric acid | Nitro | Picric acid |
| Ponceau 2R | Monoazo | Xylidine ponceau |
| Ponceau 6R | Azo | Ponceau 6R |
| Ponceau B | Disazo | Biebrich scarlet |
| Ponceau de Xylidine | Monoazo | Xylidine ponceau |
| Ponceau S | Disazo | Ponceau S |
| Primula | Triarylmethane | Hoffman's violet |
| Purpurin | Anthraquinone | Purpurin |
| Pyronin B | Pyronin | Pyronin B |
| Pyronin G | Pyronin | Pyronin Y |
| Pyronin Y | Pyronin | Pyronin Y |
| Rhodamine B | Rhodamine | Rhodamine B |
| Rosanilin | Triarylmethane | Rosanilin |
| Rose bengal | Fluorone | Rose bengal |
| Saffron | Natural | Saffron |
| Safranin O | Safranin | Safranin O |
| Scarlet R | Disazo | Sudan IV |
| Scarlet red | Disazo | Sudan IV |
| Scharlach R | Disazo | Sudan IV |
| Shellac | Natural | Laccaic acid |
| Sirius red F3B | Tetrakisazo | Sirius red F3B |
| Solochrome cyanin R | Triarylmethane | Eriochrome cyanin R |
| Soluble blue | N/A | Aniline blue |
| Solvent black 3 | Disazo | Sudan black B |
| Solvent blue 38 | Phthalocyanine | Luxol fast blue MBS |
| Solvent red 23 | Disazo | Sudan III |
| Solvent red 24 | Disazo | Sudan IV |
| Solvent red 27 | Disazo | Oil red O |
| Solvent red 45 | Fluorone | Ethyl eosin |
| Solvent yellow 94 | Fluorone | Fluorescein |
| Spirit soluble eosin | Fluorone | Ethyl eosin |
| Sudan III | Disazo | Sudan III |
| Sudan IV | Disazo | Sudan IV |
| Sudan black B | Disazo | Sudan black B |
| Sulfur yellow S | Nitro | Naphthol yellow S |
| Swiss blue | Thiazin | Methylene blue |
| Tartrazine | Azo | Tartrazine |
| Thioflavine S | Thiazole | Thioflavine S |
| Thioflavine T | Thiazole | Thioflavine T |
| Thionin | Thiazin | Thionin |
| Toluidine blue | Thiazin | Toluidine blue O |
| Toluyline red | Eurhodin | Neutral red |
| Tropaeolin G | Azo | Metanil yellow |
| Trypaflavine | Acridine | Acriflavine |
| Trypan blue | Diazo | Trypan blue |
| Uranin | Fluorone | Fluorescein |
| Victoria blue 4R | Triarylmethane | Victoria blue 4R |
| Victoria blue B | Triarylmethane | Victoria blue B |
| Victoria green B | Triarylmethane | Malachite green |
| Water blue I | Triarylmethane | Water blue I |
| Water soluble eosin | Fluorone | Eosin Y ws |
| Xylidine ponceau | Monoazo | Xylidine ponceau |
| Yellowish eosin | Fluorone | Eosin Y ws |

Furthermore, a skilled artisan recognizes which stains and their related methods are useful for the characterization of particular tissues, cells, subcellular structures, and so forth, examples of which are illustrated in Table 2.

TABLE 2
INDEX OF METHODS AND STAINS

| STAIN | TISSUE/CELL |
|---|---|
| Acridine orange fluorescence (Chick stain) | Fungi |
| Acridine orange/acriflavine fluorescent Schiff | Fungi |
| Acriflavine fluorescent PAS | Carbohydrates |
| Alcian blue, Lendrum, Slidders & Fraser | Amyloid |
| Alcian yellow toluidine blue Leung & Gibbon | Helicobacter pylori |
| Anderson's alum hematoxylin | Nuclei |
| Anderson's iron hematoxylin | Acid resistant nuclear |
| | stain and others |
| Bennhold's congo red | Amyloid |
| Burns, Pennock & Stoward's Thioflavine T | Amyloid |
| Congo red fluorescence | Amyloid |
| Eastwood and Cole's Congo red | Amyloid |
| Highman's congo red | Amyloid |
| Lendrum, Slidders & Fraser's Alcian blue | Amyloid |
| Llewellyn's sirius red | Amyloid |
| Puchtler, Sweat and Levine's congo red | Amyloid |
| Stokes' congo red | Amyloid |
| Sweat and Puchtler's sirius red | Amyloid |
| Vassar & Culling's thioflavine T | Amyloid |
| Apathy's alum hematoxylin | Nuclei |
| Bennett's alum hematoxylin | Nuclei |
| Bennhold's congo red | Amyloid |
| Bensley's trichrome | Collagen, muscle |
| Böhmer's alum hematoxylin | Nuclei |
| Brillmeyer's trichrome | Collagen, muscle |
| Burns, Pennock & Stoward's thioflavine T | Amyloid |
| Bullard's alum hemxtoxylin | Nuclei |
| Carazzi's alum hematoxylin | Nuclei |
| Carbohydrates | Periodic acid fluorescent |
| | Schiff |
| Cason's trichrome | Collagen, muscle |
| Chick stain (acridine orange fluorescence) | Fungi |
| Chromic acid fluorescent Schiff | Fungi |
| Cole's alum hematoxylin | Nuclei |
| Picro-fuchsin variants | Collagen |
| Puchtler's Picro-sinius red | Collagen |
| Van Gieson's picro-fuchsin | Collagen |
| Kohashi's trichrome | Collagen, elastic |
| Möllendorf's trichrome | Collagen, muscle |
| Mollier's trichrome | Collagen, elastic |
| Paquin & Goddard's Trichrome | Collagen, elastic |
| Pasini's Trichrome | Collagen, elastic |
| Walter's Trichrome | Collagen, elastic |
| Garvey et. al. | Collagen, elastic, fibrin |
| Garvey-Movat pentachnome | Collagen, elastic, fibrin, |
| | mucin |
| Hollande's trichrome | Collagen, mitoses, |
| | keratin |
| Bensley's trichrome | Collagen, muscle |
| Cason's trichrome | Collagen, muscle |
| Gomori's trichrome | Collagen, muscle |
| Heidenhain's Azan trichrome | Collagen, muscle |
| Kricheski's trichrome | Collagen, muscle |
| Ladewig's trichrome | Collagen, muscle |
| Lee-Brown's trichrome | Collagen, muscle |
| Lillie's trichrome | Collagen, muscle |
| Mallory's trichrome | Collagen, muscle |
| Masson's trichrome, standard type | Collagen, muscle |
| Masson's trichrome, original | Collagen, muscle |
| Masson's trichrome, original variant | Collagen, muscle |
| Masson's trichrome, yellow variant | Collagen, muscle |
| Milligan's trichrome | Collagen, muscle |
| Patay's Trichrome | Collagen, muscle |
| Lendrum, Slidders & Fraser's trichrome | Connective tissue |
| Shoobridge's polychrome | Connective tissue and |
| | more |
| Congo red, Bennhold | Amyloid |
| Congo red, Eastwood and Cole | Amyloid |
| Congo red fluorescence | Amyloid |
| Conogo red, Highman | Amyloid |
| Congo red, Puchtler, Sweat and Levine | Amyloid |
| Congo red, Stokes | Amyloid |
| Crossman's trichrome | Collagen, muscle |
| Culling & Vassar's thioflavine T | Amyloid |
| Cunningham & Engel's (Gomori's) trichrome | Muscle fibres, types I |
| | and II |
| Cytology—vaginal cells (cancer screening) | Papanicolaou's alcoholic |
| | trichrome |
| Cytology—vaginal cells | Papanicolaou's trichrome |
| Debiden's alum hematoxylin | Nuclei |
| de Groot's alum hematoxylin | Nuclei |
| Delafield's alum hematoxylin | Nuclei |
| Duprès' magenta | Nuclei (red) |
| Duprès' trichrome | Collagen, muscle |
| Eastwood and Cole's Congo red | Amyloid |
| Chrlich's alum hematoxylin | Nuclei |
| Hart's Iron resorcin fuchsin | Elastic fibres |
| Humberstone's Iron resorcin dye | Elastic fibres |
| Weigert's Iron resorcin fuchsin | Elastic fibres |
| Kohashi's trichrome | Elastic fibres, collagen |
| Mollier's trichrome | Elastic fibres, collagen |
| Paquin & Goddard's Trichrome | Elastic fibres, collagen |
| Pasini's Trichrome | Elastic fibres, collagen |
| Walter's Trichrome | Elastic fibres, collagen |
| Garvey et. al. | Elastic, fibrin, collagen |
| Garvey-Movat pentachrome | Elastic, fibrin, collagen, |
| | mucin |
| Muscle fibres, types I & II | Engel & Cunningham's |
| | (Gomori's) trichrome |
| Counterstain to alum hematoxylin | Eosin, Meter's |
| Papanicolaou's alcoholic trichrome | Exfoliated vaginal cells |
| | (cancer screening) |
| Papanicolaou's trichrome | Exfoliated vaginal cells |
| Acid resistant nuclear stain and others | Faure's iron hematoxylin |
| Perls Prussian blue | Ferric iron |
| Garvey et. al. | Fibrin, elastic, collagen |
| Garvey-Movat pentachrome | Fibrin, elastic, collagen, |
| | mucin |
| Fluorescent congo red | Amyloid |
| Fluorescent Gridley (chromic acid Schiff) | Fungi |
| Fluorescent periodic acid Schiff | Carbohydrates |
| Friedländer's alum hematoxylin | Nuclei |
| Chick stain (fluorescent) | Fungi |
| Chromic acid—fluorescent Schiff | Fungi |
| Periodic acid—fluorescent Schiff | Fungi |
| Gadsdon's alum hematoxylin | Nuclei |
| Gage's alum hematoxylin | Nuclei |
| Gallego's carbol fuchsin | Nuclei, blue-black |
| Garvey's alum hematoxylin | Nuclei |
| Garvey et. al. | Elastin, fibrin, collagen |
| Garvey-Movat pentachrome | Elastic, fibrin, collagen, |
| | mucin |
| Gibbon & Leung | Heliobacter pylori |
| Gill's alum hematoxylin | Nuclei |
| Goddard & Paquin's Trichrome | Elastic fibres, collagen |
| Goldman's iron hematoxylin | Protozoa |
| Goldner's trichrome | Collagen, muscle |
| Gomori's trichrome | Collagen, muscle |
| (Gomori) Engel & Cunningham's trichrome | Muscle fibres, types I |
| | and II |
| Gridley—Fluorescent | Fungi |
| Groot's (de Groot's) alum hematoxylin | Nuclei |
| Hamilton alum hematoxylin | Nuclei |
| Hansen's iron hematoxylin | Acid resistant nuclear |
| | stain and others |
| Harris's alum hematoxylin | Nuclei |
| Harris & Power's alum hematoxylin | Nuclei |
| Hart's iron resorcin fuchsin | Elastic fibres |
| Haug's alum hematoxylin | Nuclei |
| Haythorne's trichrome | Collagen, muscle |
| Heidenhain's Azan trichrome | Collagen, muscle |
| Heidenhain's iron hematoxylin | Acid resistant nuclear |
| | stain and others |
| Held's iron hematoxylin | Acid resistant nuclear |
| | stain and others |
| Leung & Gibbon's Alcian yellow toluidine blue | Helicobacter pylori |
| Sayeed's PAS-toluidine blue | Helicobacter pylori |
| Toluidine blue Helicobacter pylori | |
| Hematoxylin formula index | |
| Anderson | Hematoxylin, alum |
| Apathy | Hematoxylin, alum |
| Bennett | Hematoxylin, alum |
| Böhmer | Hematoxylin, alum |
| Bullard | Hematoxylin, alum |
| Carazzi | Hematoxylin, alum |
| Cole | Hematoxylin, alum |
| Debiden | Hematoxylin, alum |
| De Groot | Hematoxylin, alum |
| Delafield | Hematoxylin, alum |
| Ehrlich | Hematoxylin, alum |
| Friedländer | Hematoxylin, alum |
| Gadsdon | Hematoxylin, alum |
| Gage | Hematoxylin, alum |
| Garvey | Hematoxylin, alum |
| Gill | Hematoxylin, alum |
| Hamilton | Hematoxylin, alum |
| Harris | Hematoxylin, alum |
| Harris & Power | Hematoxylin, alum |
| Haug | Hematoxylin, alum |
| Krutsay | Hematoxylin, alum |
| Kleinenberg | Hematoxylin, alum |
| Langeron | Hematoxylin, alum |
| Launoy | Hematoxylin, alum |
| Lee | Hematoxylin, alum |
| Lillie | Hematoxylin, alum |
| McLachlan | Hematoxylin, alum |
| Martinotti | Hematoxylin, alum |
| Mallory | Hematoxylin, alum |
| Mann | Hematoxylin, alum |
| Masson | Hematoxylin, alum |
| Mayer | Hematoxylin, alum |
| Papamiltiades | Hematoxylin, alum |
| Pusey | Hematoxylin, alum |
| Rawitz | Hematoxylin, alum |
| Sass | Hematoxylin, alum |
| Schmorl | Hematoxylin, alum |
| Watson | Hematoxylin, alum |
| Meter's eosin | Hematoxylin counterstain |
| Anderson | Hematoxylin, iron |
| Faure | Hematoxylin, iron |
| Goldman | Hematoxylin, iron |
| Hansen | Hematoxylin, iron |
| Heidenhain | Hematoxylin, iron |
| Held | Hematoxylin, iron |
| Janssen | Hematoxylin, iron |
| Kefalas | Hematoxylin, iron |
| Krajian | Hematoxylin, iron |
| La Manna | Hematoxylin, iron |
| Lillie | Hematoxylin, iron |
| Lillie & Earle | Hematoxylin, iron |
| Masson (Heidenhain) | Hematoxylin, iron |
| Morel & Bassal | Hematoxylin, iron |
| Murray (Heidenhain) | Hematoxylin, iron |
| Paquin & Goddard | Hematoxylin, iron |
| Rozas | Hematoxylin, iron |
| Hematoxylin van Gieson | Collagen |
| Hollande's trichrome | Mitoses, keratin, |
| | collagen |
| Highman's congo red | Amyloid |
| Humberstone's iron resorcin dye | Elastic fibres |
| Inclusions, acitophil | Laidlaw's trichrome |
| Inclusions, acidophil | Lendrum's phioxine |
| | tartrazine |
| Iron—ferric | Perls' Prussian blue |
| Iron resorcin dye—Humberstone | Elastic fibres |
| Iron resorcin fuchsin—Hart | Elastic fibres |
| Iron resorcin fuchsin—Weigert | Elastic fibres |
| Keratin, mitoses, collagen | Hollande's trichrome |
| Janssen's iron hematoxylin | Acid resistant nuclear |
| | stain and others |
| Kefalas's iron hematoxylin | Acid resistant nuclear |
| | stain and others |
| Kohashi's trichrome | Collagen, elastic |
| Koneff's trichrome | Pituitary cells |
| Krajian's iron hematoxylin | Acid resistant nuclear |
| | stain and others |
| Kricheski's trichrome | Collagen, muscle |
| Krutsay's alum hematoxylin | Nuclei |
| Kleinenberg's alum hematoxylin | Nuclei |
| Ladewig's trichrome | Collagen, muscle |
| Laidlaw's trichrome | Acidophil cell inclusions |
| Landeron's alum hematoxylin | Nuclei |
| Launoy's alum hematoxylin | Nuclei |
| Lee's alum hematoxylin | Nuclei |
| Lee-Brown's trichrome | Collagen, muscle |
| Lendrum's phloxine tartrazine | Acidophil cell inclusions |
| Lendrum & McFarlan's trichrome | Collagen, muscle |
| Lendrum, Slidders & Fraser's Alcian blue | Amyloid |
| Lendrum, Slidders & Fraser's trichrome | Connective tissue |
| Leung & Gibbon's alcian yellow-toluidine blue | Helicobacter pylori |
| Lewis & Miller's trichrome | Pituitary cells |
| Lillie 's alum hematoxylin | Nuclei |
| Lillie's trichrome | Collagen, muscle |
| Llewellyn's sirius red | Amyloid |
| McFarlane's trichrome, one-step | Collagen, muscle |
| McFarlane's trichrome #1 | Collagen, muscle |
| McFarlane's trichrome #2 | Collagen, muscle |
| McLachlan alum hematoxylin | Nuclei |
| Magenta, Duprès' | Nuclei (red) |
| Mallory's alum hematoxylin | Nuclei |
| Mallory's trichrome | Collagen, muscle |
| Mann's alum hematoxylin | Nuclei |
| Masson's alum hematoxylin | Nuclei |
| Masson's iron hematoxylin (Heidenhain) | Acid resistant nuclear |
| | stain and others |
| Masson's trichrome, standard type | Collagen, muscle |
| Masson's trichrome, original | Collagen, muscle |
| Masson's trichrome, original variant | Collagen, muscle |
| Masson's trichrome, yellow variant | Collagen, muscle |
| Martinotti's alum hematoxylin | Nuclei |
| Mayer's alum hematoxylin | Nuclei |
| Meter's eosin | Counterstain to alum |
| | hematoxylin |
| Miller & Lewis' trichrome | Pituitary cells |
| Milligan's trichrome | Collagen, muscle |
| Mitoses, keratin, collagen | Hollande's trichrome |
| Möllendorf's trichrome | Collagen, muscle |
| Mollier's trichrome | Collagen, elastic |
| Morel & Bassal's iron hematoxylin | Acid resistant nuclear |
| | stain and others |
| Movat-Garvey pentachrome | Elastic, fibrin, collagen, |
| | mucin |
| Mucin, elastic, fibrin, collagen | Garvey-Movat |
| | pentachrome |
| Murray's iron hematoxylin (Heidenhain) | Acid resistant nuclear |
| | stain and others |
| Bensley's trichrome | Muscle, collagen |
| Cason's trichrome | Muscle, collagen |
| Gomori's trichrome | Muscle, collagen |
| Heidenhain's Azan trichrome | Muscle, collagen |
| Kricheski's trichrome | Muscle, collagen |
| Ladewig's trichrome | Muscle, collagen |
| La Manna's iron hematoxylin | Acid resistant nuclear |
| | stain and others |
| Lee-Brown's trichrome | Muscle, collagen |
| Lillie & Earle's iron hematoxylin | Acid resistant nuclear |
| | stain and others |
| Lillie's iron hematoxylin | Acid resistant nuclear |
| | stain and others |
| Lillie's trichrome | Muscle, collagen |
| Mallory's trichrome | Muscle, collagen |
| Masson's trichrome, standard type | Muscle, collagen |
| Masson's trichrome, original | Muscle, collagen |
| Masson's trichrome, original variant | Muscle, collagen |
| Masson's trichrome, yellow variant | Muscle, collagen |
| Möllendorf's trichrome | Muscle, collagen |
| Milligan's trichrome | Muscle, collagen |
| Patay's Trichrome | Muscle, collagen |
| Engel & Cunningham's (Gomori's) trichrome | Muscle fibres, types |
| | I & II |
| Neutral red counterstain | Nuclei |
| Celestine blue-hemalum | Nuclei, acid resistant |
| Gallego's carbol fuchsin | Nuclei, blue-black |
| Duprès' magenta | Nuclei, red |
| Neutral red | Nuclei, red counterstain |
| Lendrum's phloxine tartrazine | Paneth cell granules |
| Papamiltiades's alum hematoxylin | Nuclei |
| Papanicolaou's alcoholic trichrome | Exfoliated vaginal cells |
| | (cancer screening) |
| Papanicolaou's trichrome | Exfoliated vaginal cells |
| PAS-toluidine blue, Sayeed | Helicobacter pylori |
| Paquin & Goddard's iron hematoxylin | Acid resistant nuclear |
| | stain and others |
| Paquin & Goddard's Trichrome | Elastic fibres, collagen |
| Pasini's Trichrome | Elastic fibres, collagen |
| Patay's Trichrome | Collagen, muscle |
| Pentachrome, Garvey-Movat | Mucin, elastic, fibrin, |
| | collagen |
| Periodic acid fluorescent Schiff | Carbohydrates |
| Perls Prussian blue | Ferric iron |
| Phloxine tartrazine—Lendrum | Acidophil cell inclusions |
| Picro-fuchsin, van Gieson | Collagen and muscle |
| Picro-fuchsin variants | Collagen and muscle |
| Pituitary cells | Koneff's trichrome |
| Pituitary cells | Lewis & Miller's |
| | trichrome |
| Pollak's trichrome | Collagen, muscle |
| Polychrome—Shoobridge | Connective tissue and |
| | more |
| Protozoa | Goldman's iron |
| | hematoxylin |
| Puchtler's Picro-sirius red | Collagen |
| Puchtler and Sweat's Sirius red | Amyloid |
| Puchtler, Sweat and Levine's congo red | Amyloid |
| Pusey's alum hematoxylin | Nuclei |
| Rawitz' alum hematoxylin | Nuclei |
| Rozas' iron hematoxylin | Acid resistant nuclear |
| | stain and others |
| Sass's alum hematoxylin | Nuclei |
| Sayeed's PAS-toluidine blue | Helicobacter pylori |
| Schmorl's alum hematoxylin | Nuclei |
| Shoobridge's polychrome | Connective tissue and |
| | more |
| Sirius red, Llewellyn | Amyloid |
| Sirius red-picric acid, Puchtler | Collagen |
| Sirius red, Sweat and Puchtler | Amyloid |
| Stokes congo red | Amyloid |
| Sweat and Puchtler's sirius red | Amyloid |
| Thioflavine T, Burns, Pennock & Stoward | Amyloid |
| Thioflavine T, Vassar & Culling | Amyloid |
| Toluidine blue | Helicobacter pylori |
| Toluidine blue alcian yellow, Leung & Gibbon | Helicobacter pylori |
| Trichrome methods—Index to methods | |
| Trichrome methods—Comparison chart | |
| Trichrome, Lendrum, Slidders & Fraser | Connective tissue |
| Vaginal cells, exfoliated (cancer screening) | Papanicolaou's alcoholic |
| | trichrome |
| Vaginal cells, exfoliated | Papanicolaou's trichrome |
| van Gieson | Collagen |
| Vassar & Culling's thioflavine T | Amyloid |
| Verhoeff—Garvey et. al. | Elastin, fibrin, collagen |
| Virus inclusions, acidophil | Laidlaw's trichrome |
| Virus inclusions, acidophil | Landrum's phloxine |
| | tartrazine |
| Wallart & Honette trichrome | Collagen, muscle |
| Walter's Trichrome | Collagen, muscle |
| Watson's alum hematoxylin | Nuclei |
| Weigert's iron resorcin fuchsin | Elastic |
| Weiss' trichrome | Collagen, muscle |

IX. Histological Preparation

Because living cells are minute and relatively translucent, little of their inner structure can be seen without applying one or more histological stains. Pathologists routinely examine tissues after the most commonly used histochemical staining of the tissues, e.g. hematoxylin and eosin staining. The processing involves a series of steps: fixation, dehydration, embedding, and subsequent sectioning with an instrument such as a microtome. These steps are time consuming and often alter the cell structure in subtle ways. For example, fixing cells with formaldehyde will preserve the general organelle structure of the cell but may destroy agents such as antigens and enzymes which are intracellularly located.

Pathologists routinely examine tissues which have been fixed in formaldehyde and embedded in paraffin wax prior to sectioning. The process requires a minimum of 24 hours, which is crucial when a diagnosis of benign or malignant cancer is at issue. Valuable time can be saved by freezing the tissue in a modified microtome, such as the cryostat, and omitting the fixation and dehydration steps required for paraffin embedding. Additionally, frozen sections will more often retain their enzyme and antigen functions. Although the use of frozen sections can reduce the processing time, it is inadequate for long term preservation of the tissues, and the formation of ice crystals within the cells destroys subcellular features. Given that frozen sections do not section as thin as paraffin, they are also thicker. This results in poor microscopic resolution and poor images of remaining subcellular structures. If time or enzyme function is critical, frozen sections are the preferred process. If subcellular detail is important, other procedures must be used. Selection of the correct procedure depends on what the analyst is analyzing. The histologist must choose among hundreds of procedures to prepare tissues in a manner that is most appropriate to the task at hand.

A. Fixation

Since cellular decomposition begins immediately after the death of an organism, biologists must fix the cells to prevent alterations in their structure through decomposition. Routine fixation involves the chemical cross-linking of proteins (to prevent enzyme action and digestion) and the removal of water to further denature the proteins of the cell. Heavy metals may also be used for their denaturing effect.

A typical laboratory procedure involves the use of an aldehyde as the primary fixative. Glutaraldehyde is used for transmission electron microscopy (TEM), and formaldehyde is used for routine light microscopy. The formaldehyde solution most often employed was originally formulated by Baker in 1944.

Baker's Formalin Fixative contains: calcium chloride 1.0 g, cadmium chloride 1.0 g, formalin, concentrated 10.0 ml, and distilled water 100.0 ml. Blocks of tissue (liver, kidney, pancreas, and so forth) of approximately 1 cm are rapidly removed from a freshly killed organism and placed in the fixative. They are allowed to remain in the fixative for a minimum of four hours but usually overnight. The longer the blocks remain in the fixative, the deeper the fixative penetrates into the block and the more protein cross-linking occurs. The fixative is therefore termed progressive. Blocks may remain in this fixative indefinitely, although the tissues will become increasingly brittle with long exposures and will be more difficult to section. While it is not recommended, sections have been cut from blocks left for years in formalin.

Formalin has lately been implicated as a causative agent for strong allergy reactions (contact dermatitis with prolonged exposure) and may be a carcinogen—it should be used with care and always in a well ventilated environment. Formalin is a 39% solution of formaldehyde gas. The fixative is generally used as a 10% formalin or the equivalent 4% formaldehyde solution.

B. Dehydration

Fixatives, such as formaldehyde, have the potential to further react with any staining procedure which may be used later in the process. Consequently, any remaining fixative is washed out by placing the blocks in running water overnight or by successive changes of water and/or a buffer. There are myriad means of washing the tissues (using temperature, pH and osmotically controlled buffers), but usually simple washing in tap water is sufficient.

If the tissues are to be embedded in paraffin or plastic, all traces of water must be removed: water and paraffin are immiscible. The removal of water is dehydration. The dehydration process is accomplished by passing the tissue through a series of increasing alcohol concentrations. The blocks of tissue are transferred sequentially to 30%, 50%, 70%, 80%, 90%, 95%, and 100% alcohols for about two hours each. The blocks are then placed in a second 100% ethanol solution to ensure that all water is removed. Note that ethanol is hydroscopic and absorbs water vapor from the air. Absolute ethanol is only absolute if steps are taken to ensure that no water has been absorbed.

C. Embedding

After dehydration, the tissues can be embedded in paraffin, nitrocellulose or various formulations of plastics. Paraffin is the least expensive and therefore the most commonly used material. More recently, plastics have come into increased use, primarily because they allow thinner sections (about 1.5 microns compared to 5-7 microns for paraffin).

D. Paraffin

For paraffin embedding, first clear the tissues. Clearing refers to the use of an intermediate fluid that is miscible with ethanol and paraffin, since these two compounds are immiscible. Benzene, chloroform, toluene or xylol are the most commonly used clearing agents, although some histologists prefer mixtures of various oils (cedarwood oil, methyl salicylate, creosote, clove oil, amyl acetate or Cellosolve). Dioxane is frequently used and has the advantage of short preparation times.

The most often used clearing agent is toluene. It is used by moving the blocks into a 50:50 mixture of absolute ethanol:toluene for two hours. The blocks are then placed into pure toluene and then into a mixture of toluene and paraffin (also 50:50). They are then placed in an oven at 56-58° C. (the melting temperature of paraffin).

The blocks are transferred to pure paraffin in the oven for 1 hour and then into a second pot of melted paraffin for an additional 2-3 hours. During this time the tissue block is completely infiltrated with melted paraffin.

Subsequent to infiltration, the tissue is placed into an embedding mold and melted paraffin is poured into the mold to form a block. The blocks are allowed to cool and are then ready for sectioning.

E. Plastic

More recent developments in the formulation of plastic resins have begun to alter the way sections are embedded. For electron microscopy that requires ultrathin sections, paraffin is simply not suitable. Paraffin and nitrocellulose are too soft to yield thin enough sections.

Instead, special formulations of hard plastics are used, and the basic process is similar to that for paraffin. The alterations involve placing a dehydrated tissue sample of about 1 mm into a liquid plastic which is then polymerized to form a hard block. The plastic block is trimmed and sectioned with an ultramicrotome to obtain sections of a few hundred Angstroms.

Softer plastics are also being used for routine light microscopy. The average thickness of a paraffin-sectioned tissue is between 7 and 10 microns. Often this will consist of two cell layers and, consequently lack definition for cytoplasmic structures. With a plastic such as Polysciences JB-4 it is possible to section tissues in the 1-3 micron range with increased sharpness. This is particularly helpful if photomicrographs are to be taken. With the decrease in section thickness, however, comes a loss of contrast, and thin sections (1 micron) usually require the use of a phase contrast microscope as well as special staining procedures.

Soft plastics can be sectioned with a standard steel microtome blade and do not require glass or diamond knives, as with the harder plastics used for EM work.

F. Sectioning

A microtome is a simple device consisting of a stationary knife holder/blade and a specimen holder which advances by pre-set intervals with each rotation of the flywheel mounted on the right hand side. In operation, it is similar to the meat and cheese slicers found within delicatessens. A control knob adjusts internal cams which advance the paraffin block with each stroke. It is relatively easy to section paraffin at 10 microns but requires a lot of skill and practice to cut at 5 microns. Since each section comes off of the block serially, it is possible to align all of the sections on a microscope slide and produce a serial section from one end of a tissue to the other.

The Ultramicrotome is the offspring of the standard microtome, in that it also is a mechanical device that involves a stationary knife (glass or diamond) and a moving specimen. The specimen, or block, is a plastic embedded tissue that advances in nanometers rather than microns. Operationally, the only difference is that smaller samples are handled, which in turn requires a binocular dissecting microscope mounted over the blade. The tissue sections are too thin to see their thickness with the naked eye, one usually estimates thickness by the color of the diffraction pattern on the section as it floats off the knife onto the surface of a water bath. The sections are also too thin to be handled directly, and they are therefore transferred with wire loops, or picked off the water directly onto an EM grid. This process requires a good light source mounted to cast the light at just the correct angle to see the color pattern.

Since the plastics are hard enough to break steel knives, freshly prepared glass knives or commercially available diamond knives are used. A glass knife costs several dollars each, while a good diamond knife will cost in excess of $3,000. Either can be permanently damaged with a single careless stroke by the operator. Diamond knives are used in research laboratories by trained technicians because they have the advantage of a consistent knife edge (unlike glass which varies with each use) and can last for years if treated properly. They can usually be resharpened several times before discarding.

To minimize vibrations (which lead to uneven sections) ultramicrotomes are cast in heavy metal, are mounted on shock absorbent tables and, preferably, kept in draft free environments of relatively constant temperature. To further minimize vibrations, some manufacturers have replaced the block's mechanical advance mechanism with a thermal bar, which advances the tissue by heating a metal rod. This can be exquisitely precise and is the ultimate in thin sectioning. Of course with this advancement comes increased cost and maintenance, and decreased ability to withstand rough treatment. The mechanically advanced ultramicrotome remains as the workhorse of the cell biology laboratory.

G. The Cryostat

Whether the sectioning is performed with a microtome or an ultramicrotome, one of the major delays in preparing a tissue section is the time required to dehydrate and embed the tissue. This can be overcome by direct sectioning of a frozen tissue. Typically a piece of tissue can be quick frozen to about −15 to −20° C. (for light microscopic work) and sectioned immediately in a device termed a cryostat. The cryostat is merely a microtome mounted within a freezer box.

A piece of tissue is removed from an organism, placed onto a metal stub and covered with a viscous embedding compound to keep it in a form convenient for sectioning. The stud and tissue are placed within the cryostat and quick frozen. This method has the advantage of speed, maintenance of most enzyme and immunological functions (fixation is unnecessary) and relative ease of handling (far fewer steps to manipulate). It has the disadvantage that ice crystals formed during the freezing process will distort the image of the cell (bursting vacuoles and membranes for example) and the blocks tend to freeze-dry or sublimate. Thus, the blocks must be used immediately and great care must be taken to guard against induced artifact from the freezing process.

When temperature-sensitive (or lipid-soluble) molecules are to be studied, or where speed is of the essence (such as pathological examination during an operation) this is the preferred method. Sectioning operation with the cryostat is similar to that of the microtome, with the exception that one handles single frozen sections and thus all operations must be handled at reduced temperatures.

X. Laser Capture Microdissection

Developments in gene sequencing and amplification techniques, among others, now allow detailed molecular analysis of normal as well as diseased samples. The efficacy of these sophisticated genetic testing methods, however, depends on the purity and precision of the cell populations being analyzed. Simply homogenizing large tissue samples results in an impure combination of healthy and diseased cells or the cells of different populations. Using mechanical tools to manually separate cells of interest from the histologic section is time-consuming and extremely labor-intensive. None of these methods offers the ease, precision and efficiency necessary for modern molecular diagnosis.

The process of laser capture microdissection (LCM) circumvents many problems in the art regarding accuracy, efficiency and purity. A laser beam focally activates a special transfer film which bonds specifically to cells identified and targeted by microscopy within the tissue section. The transfer film with the bonded cells is then lifted off the thin tissue section, leaving all unwanted cells behind (which would contaminate the molecular purity of subsequent analysis). The transparent transfer film is applied to the surface of the tissue section. Under the microscope, the diagnostic pathologist or researcher views the thin tissue section through the glass slide on which it is mounted and chooses microscopic clusters of cells to study. When the cells of choice are in the center of the field of view, the operator pushes a button which activates a near IR laser diode integral with the microscope optics. The pulsed laser beam activates a precise spot on the transfer film immediately above the cells of interest. At this precise location the film melts and fuses with the underlying cells of choice. When the film is removed, the chosen cell(s) are tightly held within the focally expanded polymer, while the rest of the tissue is left behind. This allows multiple homogeneous samples within the tissue section or cytological preparation to be targeted and pooled for extraction of molecules and analysis.

In a commercial system, such as with the instruments and methods of Arcturus (Mountain View, Calif.) (http://www.arctur.com/), the film is permanently bonded to the underside of a transparent vial cap. A mechanical arm precisely positions the transfer surface onto the tissue. The microscope focuses the laser beam to discrete sizes (presently either 30 or 60 micron diameters), delivering precise pulsed doses to the targeted film. Targeted cells are transferred to the cap surface, and the cap is placed directly onto a vial for molecular processing. The size of the targeting pulses is selected by the operator. The cells adherent to the film retain their morphologic features, and the operator can verify that the correct cells have been procured.

Examples of LCM with, for example, breast tissue include those available at http://www.arctur.com/technology/1cm_examples/ex_breast.html.

Methods regarding the specific preparations and techniques associated with LCM are well known in the art and are provided at (http://www.arctur.com/technology/protocols.html), including: Paraffin-Embedded Tissue, Frozen Tissue, White Blood Cell Cytospin, De-Paraffinization of Tissue Sections, Hematoxylin and Eosin Staining, Immunohistochemical Staining (IHC), Intercalator Dye Staining (Fluorescence), Methyl Green Staining, Nuclear Fast Red Staining, and Toluidine Blue O Staining.

An example of Laser Capture Microdissection steps, particularly for use with Acturus instruments, includes the following:

1. Prepare. Follow routine protocols for preparing a tissue or smear on a standard microscope slide. Apply a Prep Strip™ to flatten the tissue and remove loose debris prior to LCM.

2. Place. Place a CapSure™ HS onto the tissue in the area of interest. The CapSure™ HS is custom designed to keep the transfer film out of contact with the tissue.

3. Capture. Pulse the low power infrared laser. The laser activates the transfer film which then expands down into contact with the tissue. The desired cell(s) adhere to the CapSure™ HS transfer film.

4. Microdissect. Lift the CapSure™ HS film carrier, with the desired cell(s) attached to the film surface. The surrounding tissue remains intact.

5. Extract. Snap the ExtracSure™ onto the CapSure™ HS. The ExtracSure™ is designed to accept low volumes of digestion buffer while sealing out any non-selected material from the captured cells. Pipette the extraction buffer directly into the digestion well of the ExtracSure™. Place a microcentrifuge tube on top.

6. Analyze. Invert the microcentrifuge tube. After centrifuging, the lysate will be at the bottom of the tube. The cell contents, DNA, RNA or protein, are ready for subsequent molecular analysis.

XI. Enzymes and Nucleic Acids: Modifying Enzymes

In specific embodiments of the present invention, an enzyme, such as one described as follows are utilized in the methods of the present invention, including a kit for the methods.

A. Restriction Enzymes

Examples of restriction enzymes are provided in the following Table 3.

TABLE 3
RESTRICTION ENZYMES

| | |
|---|---|
| AatII | GACGTC |
| Acc65 I | GGTACC |
| Acc I | GTMKAC |
| Aci I | CCGC |
| Acl I | AACGTT |
| Afe I | AGCGCT |
| Afl II | CTTAAG |
| Afl III | ACRYGT |
| Age I | ACCGGT |
| Ahd I | GACNNNNNGTC |
| Alu I | AGCT |
| Alw I | GGATC |
| AlwN I | CAGNNNCTG |
| Apa I | GGGCCC |
| ApaL I | GTGCAC |
| Apo I | RAATTY |
| Asc I | GGCGCGCC |
| Ase I | ATTAAT |
| Ava I | CYCGRG |
| Ava II | GGWCC |
| Avr II | CCTAGG |
| Bae I | NACNNNNGTAPyCN |
| BamH I | GGATCC |
| Ban I | GGYRCC |
| Ban II | GRGCYC |
| Bbs I | GAAGAC |
| Bbv I | GCAGC |
| BbvC I | CCTCAGC |
| Bcg I | CGANNNNNNTGC |
| BciV I | GTATCC |
| Bcl I | TGATCA |
| Bfa I | CTAG |
| Bgl I | GCCNNNNNGGC |
| Bgl II | AGATCT |
| Blp I | GCTNAGC |
| Bmr I | ACTGGG |
| Bpm I | CTGGAG |
| BsaA I | YACGTR |
| BsaB I | GATNNNNATC |
| BsaH I | GRCGYC |
| Bsa I | GGTCTC |
| BsaJ I | CCNNGG |
| BsaW I | WCCGGW |
| BseR I | GAGGAG |
| Bsg I | GTGCAG |
| BsiE I | CGRYCG |
| BsiHKA I | GWGCWC |
| BsiW I | CGTACG |
| Bsl I | CCNNNNNNNGG |
| BsmA I | GTCTC |
| BsmB I | CGTCTC |
| BsmF I | GGGAC |
| Bsm I | GAATGC |
| BsoB I | CYCGRG |
| Bsp1286 I | GDGCHC |
| BspD I | ATCGAT |
| BspE I | TCCGGA |
| BspH I | TCATGA |
| BspM I | ACCTGC |
| BsrB I | CCGCTC |
| BsrD I | GCAATG |
| BsrF I | RCCGGY |
| BsrG I | TGTACA |
| Bsr I | ACTGG |
| BssH II | GCGCGC |
| BssK I | CCNGG |
| Bst4C I | ACNGT |
| BssS I | CACGAG |
| BstAP I | GCANNNNNTGC |
| BstB I | TTCGAA |
| BstE II | GGTNACC |
| BstF5 I | GGATGNN |
| BstN I | CCWGG |
| BstU I | CGCG |
| BstX I | CCANNNNNNTGG |
| BstY I | RGATCY |
| BstZ17 I | GTATAC |
| Bsu36 I | CCTNAGG |
| Btg I | CCPuPyGG |
| Btr I | CACGTG |
| Cac8 I | GCNNGC |
| Cla I | ATCGAT |
| Dde I | CTNAG |
| Dpn I | GATC |
| Dpn II | GATC |
| Dra I | TTTAAA |
| Dra III | CACNNNGTG |
| Drd I | GACNNNNNNGTC |
| Eae I | YGGCCR |
| Eag I | CGGCCG |
| Ear I | CTCTTC |
| Eci I | GGCGGA |
| EcoN I | CCTNNNNNAGG |
| EcoO109 I | RGGNCCY |
| EcoR I | GAATTC |
| EcoRV | GATATC |
| Fau I | CCCGCNNNN |
| Fnu4H I | GCNGC |
| Fok I | GGATG |
| Fse I | GGCCGGCC |
| Fsp I | TGCGCA |
| Hae II | RGCGCY |
| Hae III | GGCC |
| Hga I | GACGC |
| Hha I | GCGC |
| Hinc II | GTYRAC |
| Hind III | AAGCTT |
| Hinf I | GANTC |
| HinPl I | GCGC |
| Hpa I | GTTAAC |
| Hpa II | CCGG |
| Hph I | GGTGA |
| Kas I | GGCGCC |
| Kpn I | GGTACC |
| Mbo I | GATC |
| Mbo II | GAAGA |
| Mfe I | CAATTG |
| Mlu I | ACGCGT |
| Mly I | GAGTCNNNNN |
| Mnl I | CCTC |
| Msc I | TGGCCA |
| Mse I | TTAA |
| Msl I | CAYNNNNRTG |
| MspAl I | CMGCKG |
| Msp I | CCGG |
| Mwo I | GCNNNNNNNGC |
| Nae I | GCCGGC |
| Nar I | GGCGCC |
| Nci I | CCSGG |
| Nco I | CCATGG |
| Nde I | CATATG |
| NgoMI V | GCCGGC |
| Nhe I | GCTAGC |
| Nla III | CATG |
| Nia IV | GGNNCC |
| Not I | GCGGCCGC |
| Nru I | TCGCGA |
| Nsi I | ATGCAT |
| Nsp I | RCATGY |
| Pac I | TTAATTAA |
| PaeR7 I | CTCGAG |
| Pci I | ACATGT |
| PflF I | GACNNNGTC |
| PflM I | CCANNNNNTGG |
| Ple I | GAGTC |
| Pme I | GTTTAAAC |
| Pml I | CACGTG |
| PpuM I | RGGWCCY |
| PshA I | GACNNNNGTC |
| Psi I | TTATAA |
| PspG I | CCWGG |
| PspOM I | GGGCCC |
| Pst I | CTGCAG |
| Pvu I | CGATCG |
| Pvu II | CAGCTG |
| Rsa I | GTAC |
| Rsr II | CGGWCCG |
| Sac I | GAGCTC |
| Sac II | CCGCGG |
| Sal I | GTCGAC |
| Sap I | GCTCTTC |
| Sau3A I | GATC |
| Sau96 I | GGNCC |
| Sbf I | CCTGCAGG |
| Sca I | AGTACT |
| ScrF I | CCNGG |
| SexA I | ACCWGGT |
| SfaN I | GCATC |
| Sfc I | CTRYAG |
| Sfi I | GGCCNNNNNGGCC |
| Sfo I | GGCGCC |
| SgrA I | CRCCGGYG |
| Sma I | CCCGGG |
| Sml I | CTYRAG |
| SnaB I | TACGTA |
| Spe I | ACTAGT |
| Sph I | GCATGC |
| Ssp I | AATATT |
| Stu I | AGGCCT |
| Sty I | CCWWGG |
| Swa I | ATTTAAAT |
| Taq I | TCGA |
| Tfi I | GAWTC |
| Tli I | CTCGAG |
| Tse I | GCWGC |
| Tsp45 I | GTSAC |
| Tsp509 I | AATT |
| TspR I | CAGTG |
| Tth111 I | GACNNNGTC |
| Xba I | TCTAGA |
| Xcm I | CCANNNNNNNNNTG |
| | G |
| Xho I | CTCGAG |
| Xma I | CCCGGG |
| Xmn I | GAANNNNTTC |

The term “restriction enzyme digestion” of DNA as used herein refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 μg of plasmid or DNA fragment is used with about 1-2 units of enzyme in about 20 μl of buffer solution. Appropriate buffers and substrate amounts for particular restriction enzymes are specified by the manufacturer. Incubation of about 1 hour at 37° C. is ordinarily used, but may vary in accordance with the supplier's instructions. After incubation, protein or polypeptide is removed by extraction with phenol and chloroform, and the digested nucleic acid is recovered from the aqueous fraction by precipitation with ethanol. Digestion with a restriction enzyme may be followed with bacterial alkaline phosphatase hydrolysis of the terminal 5′ phosphates to prevent the two restriction cleaved ends of a DNA fragment from “circularizing” or forming a closed loop that would impede insertion of another DNA fragment at the restriction site. Unless otherwise stated, digestion of plasmids is not followed by 5′ terminal dephosphorylation. Procedures and reagents for dephosphorylation are conventional as described in Sambrook et al. (1989).

B. Polymerases and Reverse Transcriptases

Thermostable DNA Polymerases

OmniBase™ Sequencing Enzyme

Pfu DNA Polymerase

Taq DNA Polymerase

Taq DNA Polymerase, Sequencing Grade

Taq Mini Kit

TaqBead™ Hot Start Polymerase, 1.25 u/bead, Nonbarrier

Tfl DNA Polymerase

Tfl DNA Polymerase Mini Kits

Tli DNA Polymerase

Tth DNA Polymerase

DNA Polymerases

DNA Polymerase I, Klenow Fragment, Exonuclease Minus

DNA Polymerase I

DNA Polymerase I Large (Klenow) Fragment

DNA Polymerase I Large (Klenow) Fragment Mini Kit

Terminal Deoxynucleotidyl Transferase

T4 DNA Polymerase

RNA Polymerases

SP6 RNA Polymerase

T3 RNA Polymerase

T7 RNA Polymerase

Reverse Transcriptases

AMV Reverse Transcriptase

M-MLV Reverse Transcriptase

C. DNA/RNA Modifying Enzymes

Ligases

T4 DNA Ligase

T4 RNA Ligase

Kinases

T4 Polynucleotide Kinase

Nucleases

Exonuclease III

Mung Bean Nuclease

Nuclease BAL 31

Ribonuclease H

RNase ONETM Ribonuclease

RQ1 RNase-Free DNase

S1 Nuclease

Phosphatases

Alkaline Phosphatase, Calf Intestinal (CIAP)

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Amplification of Sense and Antisense RNA by Terminal Continuation

The method of terminal continuation allows for the efficient linear amplification of nucleic acids, including sense and antisense strand RNA. Current methods of RNA amplification either distort the quantitative relationship between gene populations or are limited to inefficiently synthesizing antisense RNA.

mRNA is purified using standard methods that prevent RNA degradation. Small amounts of mRNA, as low as picogram amounts, are used as the target nucleic acid strand. First strand synthesis primers containing poly(dT) and an SP6 transcriptional promoter at its 5′ end, terminal continuation oligononucleotides having the T7 transcriptional promoter sequence and three deoxyguanines at the 3′ end, and reverse-transcriptase enzyme are added to the mRNA. The poly(dT) sequence of the first strand synthesis primer anneals to the poly(A) tail of mRNA, serving as a primer for reverse-transcriptase to synthesize first strand cDNA. At the 3′ end of the first strand cDNA, reverse-transcriptase adds the nucleic acid sequence that is complementary to the terminal continuation oligonucleotide, in this case, the complementary sequence to T7 transcriptional promoter-GGG (FIG. 3). The 5′ end of first strand cDNA has the SP6 promoter followed by a poly(T) stretch, as this sequence was used as the primer for first strand synthesis.

RNA digestion or heat denaturation is used to disassociate the mRNA with the first strand cDNA. mRNA::first strand cDNA complex may now be isolated for use as a reagent in other biological applications. To the disassociated first strand cDNA, the terminal continuation oligonucleotide is added to serve as a primer for Taq polymerase for second strand cDNA synthesis. The terminal continuation primer anneals to its complementary sequence at the 3′ end of first strand cDNA. The Taq polymerase then synthesizes the second strand cDNA, which contains the sequence of the terminal continuation primer at its 5′ end and the complementary sequence of first strand cDNA. Thus at this point, a double-strand cDNA molecule has been formed which contains a functional T7 transcriptional promoter at the 5′ end of second strand cDNA and a functional SP6 transcriptional promoter at the 5′ end of first strand cDNA.

In vitro RNA transcription is conducted using the second strand cDNA and/or first strand cDNA as a template. With the addition of T7 polymerase and rNTPs, T7 polymerase initiates transcription at the 5′ end of second strand cDNA. With the second strand cDNA as the template of transcription, sense strand RNA is amplified. With the addition of SP6 polymerase, SP6 polymerase initiates transcription from the 5′ end of first strand cDNA. With the first strand cDNA as the template of transcription, antisense strand RNA is amplified. The amplified RNA can be reverse-transcribed to generate abundant amounts of cDNA. In addition, the amplified sense strand RNA may be used as templates for in vitro translation.

### Example 2

### Method to Linearly Amplify RNA

The amplification of RNA through in vitro transcription has the advantage over RT-PCR because of its ability to better preserve the quantitative relationship between different genetic signals, which is a feature that makes it a preferred method for gene profiling.

The key step of the procedure is the synthesis of ds cDNA template for the subsequent RNA transcription. Traditional methods use either self-priming (Van Gelder et al., 1990; Eberwine et al., 1992; U.S. Pat. No. 5,545,522) or replacement methods to prime the second strand synthesis. However, both suffer from the low efficiency in generating ds cDNA template for subsequent RNA transcription. The methods and materials of the present invention significantly increase the efficiency of ds cDNA template synthesis. The flow chart of FIG. 1, describes the “terminal continuation” technology for the synthesis of ds cDNA template. Some obvious modifications in protocol, e.g., the choice of first or/and second primer to attach the promoters, the choice of different promoters, the reduction or addition of functional sequences, such as restriction enzyme digestion sequences or protein synthesis starting sequences, all fall within the scope of the present invention.

Step 1. First Strand Synthesis

10 pmol of first primer (oligo d(T) primer)

5′-d(T)24VN-3′ (where V=G or A or C; N=G or A or T or C) and

10 pmol of second primer (terminal continuation (TC) primer)-

5′d(AAACGACGGCCAGTGAATTGTAATACGACTCACTATAGGCG CDAGAG)r(GGGG)-3′ (SEQ ID NO: 4) (TC primer contains a T7 RNA synthesis promoter) are annealed to total RNA from a single neuron (containing approximately 0.1-1 pg mRNA) in volume of 7 μl of RNase free water, by heating the mixture at 85° C. for 2 minutes, followed by cooling on ice for at least 2 minutes. First-strand cDNA synthesis is initiated by adding to the annealed primer-RNA 200 units of M-MLV RNase H- reverse transcriptase in a final volume of 20 μl, containing 50 mM Tris-HCl, pH 8.3, 75 mM KCl; 3 mM MgCl₂; 1 mM DTT; and 1 mM each of dATP, dGTP, dCTP, and dTTP. The first strand synthesis reaction is incubated at 42° C. for 60 minutes.

Primers other than listed above, e.g. an oligo d(T) primer containing a RNA synthesis promoter, or short primers of random sequences can also be used as the first primer in the reaction; and an oligo with a RNA synthesize promoter other than T7 promoter or a primer with random sequence at its 5′ and multiple rG at 3′ can be used as TC primers.

Step 2. Second Strand cDNA Synthesis

Second strand cDNA synthesis is initiated by mixing 5 units of Taq DNA polymerase with the first strand synthesis reaction in a final volume of 100 μl, containing 1 unit of RNase H, 25 mM Tris-HCl, pH 8.3, 65 mM KCl, and 2 mM MgCl₂. The reaction is performed in a thermocycler with these sequential temperature changes; 37° C. for 10 minutes, 95° C. for 3 minutes, 50° C. for 3 minutes and, finally, 75° C. for 30 minutes. The reaction is terminated by extracting with phenol/chloroforn/isoamyl alcohol (25:24:1) once and the synthesized ds cDNA is precipitated with 2.5 M of ammonium acetate (final concentration), and 1 ml cold 100% ethanol. Ten jig linear acrylamide is added to facilitate the precipitation. The ds cDNA is pelleted by centrifugation at 14,000 rpm at room temperature in a tabletop microfuge and the pellet then air-dried. The cDNA is then drop dialyzed to rid excess salt for 2 hours at room temperature and the final volume adjusted as determined by the desired by downstream experiments.

Step 3. In vitro RNA amplification. In a suitable condition, each ds cDNA template is used to transcribe hundreds to thousands copies of RNA through in vitro transcription, which leads to the amplification of the original genetic signals. In vitro transcription was done by adding 1,000 units of T7 RNA polymerase to the reaction mixture in a final volume of 20 μl containing 40 mM Tris-HCl, pH 7.5, 7 mM MgCl₂, 10 mM NaCl, 2 mM sperrnidine, 5 mM DTT, 20 units of RNase inhibitor and 0.5 mM of each of ATP, GTP, CTP and UTP. The reaction is done at 37° C. for four hours. In some applications, the transcribed RNA was subjected to the further amplification before the downstream processing. In this situation, the above steps 1, 2 and 3 can be repeated at least once.

### Example 3

### Detection of Weak Genetic Signals by Hybridization of Amplified RNA Probes

Methods and materials of the present invention are used effectively to generate RNA probes to detect genetic signals. Following the amplification steps illustrated in Example 1, genetic signals, especially weak signals, are substantially amplified. Therefore, the signals too weak to be detected without amplification can be detected readily. This feature is especially useful when the supply of starting material is limited, e.g. clinical samples or specific cell types such as tumor cells or discrete neuronal populations. It will be apparent to those skilled in the art that each individual step or material used for the procedure, e.g. reporter group used to label RNA probe, supporting materials or hybridization procedures, can be varied without changing the final result of the procedure. Any such variations in the preferred protocol, which are based on using methods and materials of the subject invention, are within the scope of the invention.

Step 1. Generation of Amplified Hybridization Probes

The generation of amplified RNA probes requires first converting original RNA population into ds cDNA template as described in Example 1. In some applications, RNA was directly labeled during the transcription by incorporating radioisotope, e.g. 40 μCi ³³P-UTP, to generate the RNA probe for hybridization. To increase the specific activity of the labeled RNA probe, unlabeled UTP is adjusted to final concentration of 5 μM.

In an alternative embodiment, a cDNA probe is generated with a reverse transcription procedure in the presence of labeled deoxyribonucleic acid. Briefly, 0.5 μg random hexomers hexamers are annealed to amplified RNA in volume of 7 μl of RNase free water, by heating the mixture at 72° C. for 2 minutes, followed by cooling on ice for 2 minutes. The reverse transcription is initiated by adding into the annealed primer:RNA mixture 200 units of M-MLV RNase H-reverse transcriptase in a final volume of 20 μl, containing 50 mM Tris-HCl, pH 8.3, 75 mM KCl; 3 mM MgCl₂; 1 mM DTT; 6.5 μM fluorescent Cy3 Cy5 labelled dCTP, 1 mM each of dATP, dGTP, dTTP, and 0.1 mM dCTP. The probe synthesis reaction is incubated at 42° C. for 60 minutes. One unit of RNase H is then added, and the reaction mixture is incubated at 37° C. for 10 minutes. The probe is purified using a Qiagen commercially available PCR purification kit.

Step 2. Hybridization

In a specific embodiment, the generated RNA probes are used in reverse Northern hybridization analysis. Genes of known DNA sequences are arrayed or directly spotted on a solid support, which is subjected to prehybridization for four hours at 42° C. prior to addition of the RNA probe. When a nylon membrane is used, the pre-hybridization step is performed in a final volume of 10 ml prehybridization solution containing 50% formamide, 6×SSPE, 5×Denhardt's solution, 0.1% SDS and 10 mM Na₂PPi and 200 ng/ml salmon sperm DNA. After a labeled RNA probe is added into the prehybridization solution, the hybridization continues for another eighteen hours. The membrane blots are washed sequentially with 10 ml 2×SSC, 0.1% SDS, 1×SSC, 0.1% SDS and 0.5 SSC, 0.1% SDS at 42° C. for 15 minutes. Hybridization signal intensity is detected by a phosphorimager.

In an alternative embodiment, Cy3 or Cy5 labeled probes are used in cDNA microarray analysis. When glass slides are used, the prehybridization step is performed by immersing the glass slides in 0.2% SDS in room temperature for 5 minutes, 3 times followed by H₂O at 95° C. for 2 minutes and drying with nitrogen gas. The hybridization is performed in 5×SSC, 0.2% SDS, 65° C. for four hours. The slides are washed sequentially with 3×SSC, 0.2% SDS for 5 minutes at 65° C., 0.1×SSC 0.2% SDS for 5 minutes at room temperature and 0.1×SSC and room temperature for 30 seconds. The slides are dried and imaged using a laser scanning apparatus.

### Example 4

### RNA Amplification Based cDNA Library Construction

Conventional procedures for constructing a cDNA library starts with obtaining an mRNA population from tissues of interest, which is then converted into first strand cDNA by reverse transcription. Double stranded cDNA can usually be generated through a single step second strand synthesis or PCR when an amplification of the cDNA is necessary. However, the conventional procedures are not suitable for constructing a cDNA library from a homogeneous cell population, especially when the quantity of starting materials is limited. Although some genetic signals can be amplified by PCR, genes of low copy number in a minority cell population of a tissue can easily be obscured and/or lost after the amplification of PCR. With the methods of the present invention, minute amounts of mRNAs harvested from a variety of different tissues can be amplified linearly before constructing a library. Therefore, cell specific genes, especially genes of low copy number, are enriched and subsequently identified.

The amplified RNA population is generated through the three steps illustrated in Example 1, which was subjected to the following further treatment. (illustrated in FIG. 4)

Step 1. First-Strand Synthesis-Terminal Continuation

100 ng First primer 5′-d(CCCAGAATTC(T)₂₀VN)-3′ (SEQ ID NO: 5)

100 ng terminal continuation primer 5′-d(GGGCAATTCAAGCCTA)r(GGG)-3′ (SEQ ID NO: 6) are annealed to the amplified RNA in a volume of 7 μl RNase/DNase free water by heating the mixture for 2 minutes at 85° C., followed by cooling on ice for 2 minutes. First-strand cDNA synthesis is initiated by mixing the annealed primer-RNA with 200 units of M-MLV RNase H reverse transcriptase in a final volume of 20 μl, containing 50 mM Tris-HCl, pH 8.3, 75 mM KCl; 3 MM MgCl₂; 1 mM DTT; and 1 mM each of dATP, dGTP, dCTP, and dTTP. The first strand synthesis reaction is incubated at 42° C. for 60 minutes.

Step 2. Second Strand cDNA Synthesis

The second-strand cDNA synthesis is initiated by mixing 5 units of Taq DNA polymerase with the first-strand synthesis reaction in a final volume of 100 μl, containing 1 unit of RNase H, 25 mM Tris-HCl, pH 8.3, 65 mM KCl, and 2 mM MgCl₂. The reaction is performed in a thermocycler with the following steps; 37° C. for 10 minutes, 95° C. for 3 minutes, 50° C. for 3 minutes, and 75° C. for 30 minutes. Five units of EcoR I restriction enzyme are then added to the reaction and incubated in room temperature for 30 minutes. The reaction is terminated by extraction with phenol:chloroform once and the synthesized ds cDNA is precipitated by adding 2.5 M of ammonium acetate (final concentration), and 1 ml cold 100% ethanol. Ten mg linear acrylamide is added to facilitate precipitation. The pellet is washed once with 1 ml 95% ethanol and air-dried.

Step 3. Ligation the Double Stranded DNA into a Suitable Cloning Vector

The EcoR I restriction enzyme digested ds cDNA is ligated into a suitable cloning vector using standard protocols (e.g., lambda ZAP II vector (Stratagene; La Jolla, Calif.) and packaged with Gigapack III gold Extract (Stratagene; La Jolla, Calif.) according to manufacturer's instructions).

### Example 5

### Microarray Detection using Methods of the Present Invention

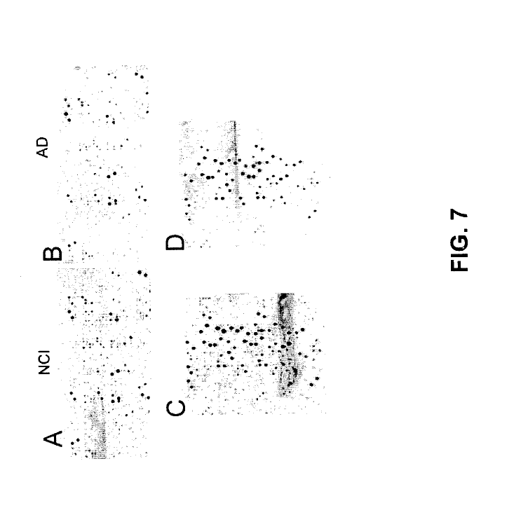

FIG. 7 illustrates how the methods of the present invention are useful for amplification and detection using high-density arrays. In FIG. 7, an Incyte life grid microarray having approximately 8,400 ESTs was obtained from Ambion (Austin, Tex.). FIG. 7 shows significant signal intensity and distribution, as well as some poignant differences between normal (7A and 7C; NCI) and Alzheimers's disease (7B and 7D; AD).

### Example 6

### Comparison of Methods of the Present Invention

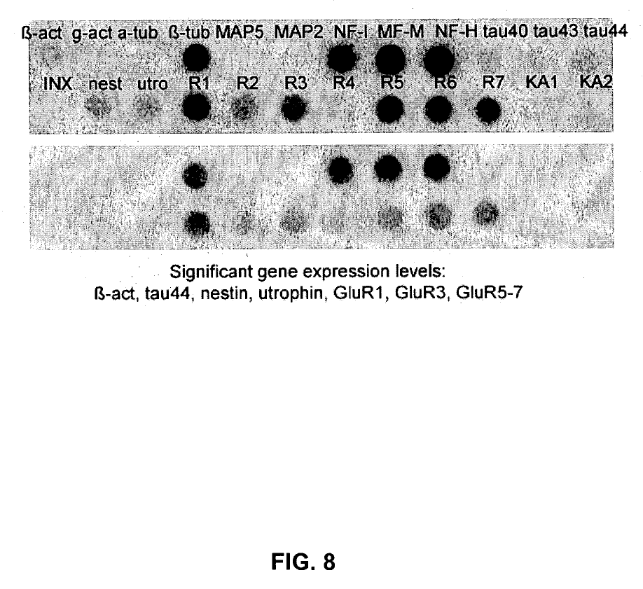

FIG. 8 illustrates the comparison of two identical aliquots of RNA extracted from the same tissue section amplified by methods of the present invention versus aRNA methods in the art (Van Gelder et al., 1990; Eberwine et al., 1992; Miyashiro et al., 1994). The relative hybridization signal intensity of the low, moderate, and higher expressing genes using the new methodology of the present invention are improved using the new methods of the present invention compared to aRNA methods known in the art. All other steps in the procedure were performed identically, such as hybridization time, identical washing regimens, and source of the array. Significant gene expression levels are detected for β-act, tau44, nestin, utrophin, GluR1, GluR3, and GluR5-7.

### Example 7

### Amplification of RNA

Materials and methods for this example are as follows:

RNA preparation. RNAs, either total or mRNAs, are extracted from tissues, single cells, or bodily fluids (Van Deerlin et al., 2002; Ginsberg et al., 2001). The TC method is especially useful when employed in conjunction with single cell (or population cell) laser capture microdissection or microaspiration. For optimal extraction from fixed tissues, single cells or populations are incubated in 250 μl of Proteinase K solution (Ambion, 50 μg/ml) for 12 hours at 37° C. prior to extraction. RNA can be extracted using conventional organic methods (e.g. Trizol reagent, Invitrogen) or semi-automated magnetic mRNA extraction methods (e.g., KingFisher, ThermoLabsystems).

RNA amplification. Amplification of genetic signals includes synthesizing first strand cDNA complementary to the RNA template, subsequently generating second strand cDNA complementary to the first strand cDNA, and finally in vitro RNA transcription using the ds cDNA as template. For synthesis of the first strand cDNA complementary to template mRNA, two oligonucleotide primers are used, a poly d(T) primer and a TC primer. The poly d(T) primer used in TC RNA amplification is similar to conventional primers that exploit the poly A⁺ sequence present on most mRNAs, typically containing 24 TTPs (plus a bacteriophage promoter sequence for antisense amplification; see Table 4).

### Table 4. Oligonucleotide Sequences Utilized for the Poly D(T) and TC Primers for the TC RNA Amplification Method

Antisense RNA Orientation

| | | |
|---|---|---|
| poly d(T)-T7 primer (66 bp): | 3′- AAA CGA CGG CCA GTG AAT TGT AAT ACG ACT | (SEQ ID NO:5) |
| | CAC TAT AGG CGC TTT TTT TTT TTT TTT TTT TTT TTT -5′ | |
| TC primer (17 bp): | 5′- TAT CAA CGC AGA GTC CC -3′ | (SEQ ID NO:6) |

Sense RNA Orientation

| | | |
|---|---|---|
| poly d(T) primer (18 bp): | 3′- TTT TTT TTT TTT TTT TTT -5′ | (SEQ ID NO:7) |
| TC-T7 primer (51 bp): | 5′- AAA CGA CGG CCA GTG AAT TGT AAT ACG ACT | (SEQ ID NO:8) |
| | CAC TAT AGG CGC GAG AGC CCC-3′ | |

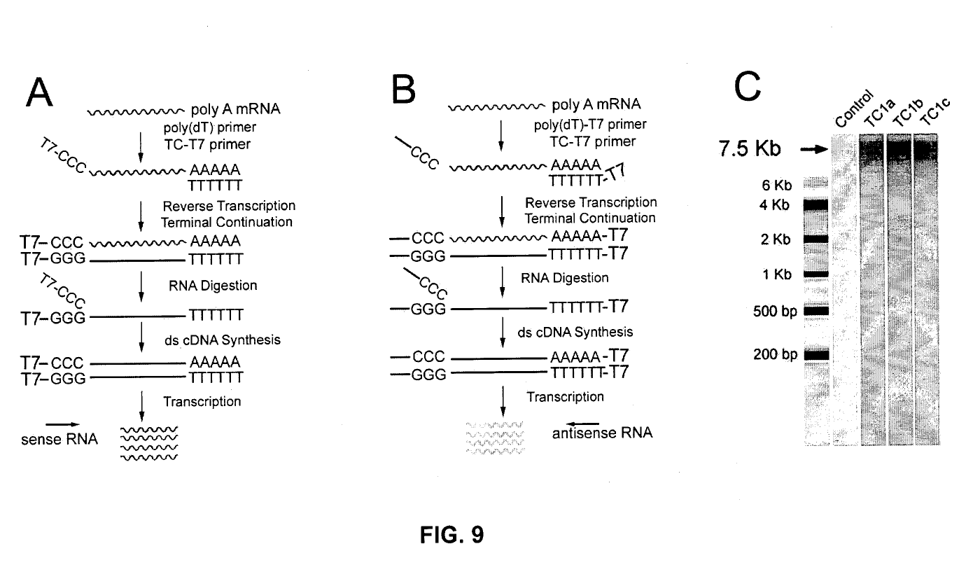

The TC primer consists essentially of two parts, an oligonucleotide sequence at the 5′ terminus and a short span of three cytosines (CTPs) at the 3′ terminus. An advantage of using this methodology is that in vitro transcription can be directed either in a ‘sense’ or ‘antisense’ (or both sense and antisense) orientation, depending on where the bacteriophage promoter(s) are attached (Table 4). Specifically, for antisense RNA amplification (similar to the conventional aRNA), the bacteriophage promoter (i.e., T7, T3, SP6) sequence is placed on the poly d(T) primer. For the novel sense orientation, the bacteriophage sequence is attached to the TC primer (FIG. 9A).

Extracted RNAs are reverse transcribed in the presence of the poly d(T) primer (100 ng/μl) and TC primer (200 ng/μl) in 1× first strand buffer (Invitrogen; Carlsbad, Calif.), 1 mM dNTPs, 5 mM DTT, 20 U of RNase inhibitor (Ambion; Austin, Tex.) and 5 U reverse transcriptase (Superscript II; Invitrogen; Carlsbad, Calif.) in a final volume of 20 μl. The synthesized single stranded (ss) cDNAs are converted into ds cDNAs by adding into the reverse transcription reaction the following: 10 mM Tris (pH 8.3), 50 mM KCl, 1.5 mM MgCl₂, 0.5 U RNase H (Invitrogen), and 5 U Taq polymerase (PE Biosystems) in a total volume of 100 μl. The samples are placed in a thermal cycler and second strand synthesis proceeds as follows: RNase H digestion step 37° C., 10 min.; denaturation step 95° C., 3 min., annealing step 50° C., 3 min; elongation step 75° C., 30 min. The reaction is terminated with 5M ammonium acetate. The samples are then extracted in phenol:chloroform:isoamyl alchohol (25:24:1) and ethanol precipitated. The cDNAs are pelleted in a tabletop centrifuge and washed once with 95% ethanol and air-dried. The cDNAs are then resuspended and drop dialyzed on 0.025 μm filter membranes (Millipore) against 50 ml of RNase-free H₂O for 2 hours. The sample is collected off the dialysis membrane and hybridization probes are synthesized by in vitro transcription using radiolabel, fluorescent, or biotin incorporation. For example, radiolabeling with ³³P occurs in the following solution: 40 mM Tris (pH 7.5), 7 mM MgCl₂, 10 mM NaCl, 2 mM spermidine, 5 mM of DTT, 0.5 mM of ATP, GTP, and CTP, 10 μM of cold UTP, 20 U of RNase inhibitor, and 40 μCi of ³³P-UTP (Amersham Biosciences). The reaction is performed at 37° C. for 4 hours. The synthesized radioisotope-labeled RNA probes are added into the prehybridization solution directly without further purification.

cDNA array analysis. Labeled probes can be used for a variety of downstream applications including expression profiling in combination with a myriad of cDNA array platforms. We typically utilize single cell microdissection in conjunction with TC RNA amplification to hybridize to custom-designed cDNA arrays consisting of (220-384) cDNAs and ESTs for analysis of neurodegeneration-related paradigms in mouse brain and human postmortem brain tissues (Ginsberg et al., 1999; Ginsberg et al., 1999; Ginsberg et al., 2000). Specifically, 1 μg of linearized cDNA purified from plasmid preparations is adhered to arrays using high-density nitrocellulose (Hybond XL, Amersham Biosciences). Each cDNA/EST on the custom-designed cDNA arrays is verified by restriction digestion and sequence analysis. Mouse, rat, and human clones are successfully employed on the arrays. Arrays are prehybridized (12 hours) and hybridized (48 hours) in a solution consisting of 6×SSPE, 5×Denhardt's solution, 50% formamide, 0.1% sodium dodecyl sulfate (SDS), and denatured salmon sperm DNA (200 μg/ml) at 42° C. in a rotisserie oven (Ginsberg et al., 2001; Ginsberg et al., 2000; Ginsberg et al., 1999). Following hybridization, arrays are washed sequentially with 2×SSC/0.1% SDS, 0.5×SSC/0.1% SDS and 0.1×SSC/0.1% SDS for 20 min each at 42° C. TC hybridization signal intensity is detected by phosphor imaging. Specific signal intensity (minus background using the empty vector pBs) of TC amplified RNA bound to each linearized cDNA is expressed as a ratio of the total hybridization signal intensity of the array, thereby minimizing variations due to differences in the specific activity of the probe and the absolute quantity of probe present. Data analyzed in this manner does not allow the absolute quantitation of mRNA levels, but generates an expression profile of the relative changes in mRNA levels. Relative changes in individual mRNAs are analyzed using ANOVA with post-hoc analysis (Newman-Keuls test) for individual comparisons. Differentially expressed genes are also clustered into functional protein categories for multivariate coordinate gene expression analysis.

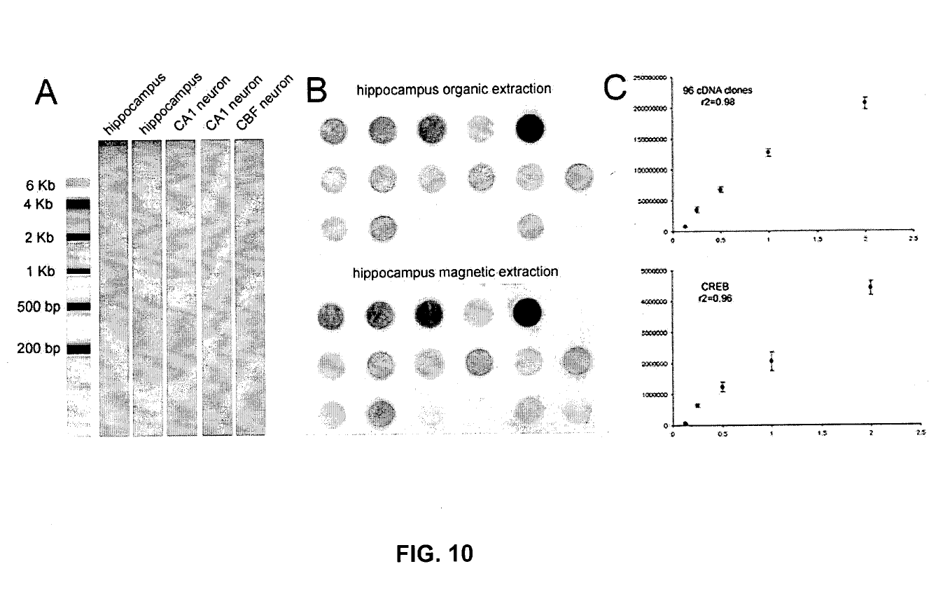

TC provides reproducible, linear RNA amplification. To evaluate the ability of the TC method to amplify RNA species, yield and size distribution profiles are estimated by bioanalysis (2100 Bioanalyzer, Agilent Technologies) using a RNA6000 LabChip (Agilent Technologies). This assay utilizes a capillary device and a sensitive fluorescent RNA dye for electrophoretic separation and detection of RNA profiles. Using a 7.5 Kb purified control poly(A⁺) obtained commercially (Invitrogen), highly reproducible, robust linear amplification is demonstrated (FIG. 9C). Concordance analysis of amplification using aliquots of the control poly(A⁺) mRNA as starting template (n=6; series run twice in triplicate) is r2=0.97, also indicates a high level of reproducibility. Amplification efficiency (as estimated using bioanalysis) of approximately 2500-3000 fold is demonstrated with the control poly(A⁺) mRNA. Amplification of approximately 1000-1500 fold is demonstrated using biological samples of RNA extracted from a variety of brain sources including post mortem hippocampus and basal forebrain (FIG. 10A). The efficiency of RNA amplification appears independent of the method of RNA extraction, as both conventional phenol:chloroform extraction and semi-automated magnetic bead extraction both yield high quality transcripts for subsequent TC RNA amplification (FIG. 10B). In addition, scatter plots demonstrate a linear relationship between TC RNA input concentration and mean hybridization signal intensity of all cDNA clones (n=96) and an individual clone (CREB is depicted) on a custom-designed cDNA array (FIG. 10C). These observations are strikingly similar to linearity data obtained by this group using an aRNA amplification methodology (Ginsberg et al., 2000).

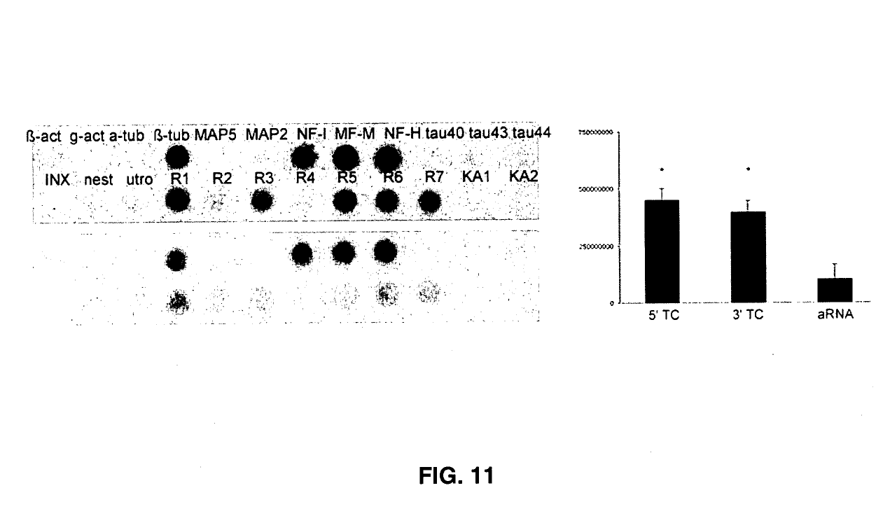

TC has increased sensitivity. The TC RNA amplification methodology produces robust and reproducible hybridization signal intensity after one round of amplification. The threshold of detection of genes with low hybridization signal intensity is also greatly increased. For example, several genes that are at the limit of detection using conventional aRNA can be readily observed with the TC method (FIG. 11A). An approximate 3.5-4 fold increase in total, normalized hybridization signal intensity is observed on custom-designed cDNA arrays (FIG. 11B). Importantly, the increased sensitivity appears greatest for genes with relative low abundance (FIG. 11A). Genes with a relatively high hybridization signal intensity display the nearly the same normalized signal value as in conventional aRNA methodology. This apparent asymptote of highly expressed genes can be readily explained by an overall increase in total hybridization signal intensity of all genes using the TC RNA amplification method. Thus, the denominator for normalization becomes larger and normalized signal values become greater for the lower expressed genes and remain approximately the same for highly expressed genes.

TC is effective in a variety of tissue sources. The TC methodology has been shown to work with total tissues as well as fixed regions such as paraffin-embedded postmortem hippocampus (FIG. 10A; lanes 1-2). Further, single cells and populations of single cells obtained through laser capture microdissection or microaspiration can be utilized with one round of amplification (FIG. 10A; lanes 3-5). Individual cells can be identified in paraffin-embedded tissues as well as fixed, frozen sectioned tissues using a variety of histochemical stains (e.g., cresyl violet, thionin, hematoxylin & eosin, and others) as well as immunohistochemical methods.

TC allows for amplification in ‘antisense’ and ‘sense’ orientations. A bacteriophage transcription promoter drives linear amplification of genetic signals, either attached to 3′ of mRNA through hybridization of the poly(A⁺) tail with a poly d(T)-promoter, similar to conventional methods, or the transcription promoter can be attached to the 5′ end of transcripts using the TC method, directing RNA synthesis in the sense direction. To date, no overall quantitative differences have been detected in total hybridization signal intensity between 3′ and 5′ TC RNA amplification reactions (FIG. 11B). However, individual genes have been identified that are expressed differentially. For example, the neurofilament genes NF-M and NF-H display a relative increase in the 3′ TC amplification as compared to the 5′ TC RNA amplication version using single neurofilament-immunoreactive CA1 pyramidal neurons from normal human hippocampus (Table 5).

### Table 5. Gene Expression Analysis of Individual, Neurofilamint-Immunoreactive CA1 Pyramidal Neurons (N=25) from Adult Human Brains (N=5 Brains; 5 CA1 Neurons Apiece) using 3′ and 5′ TC RNA Amplification Combined with Custom-Designed cDNA Arrays (230 cDNAS).

A. Classes of Transcripts that do not Vary Between 3′ and 5′ TC RNA Amplification Procedures:

acetylcholine receptors/synthesis (14), Alzheimer's disease associated genes (n=16), catehcolamine synthesis/transporters (10), cell death/transcriptional activators (n=15), cytoskeletal elements (n=20), dopamine receptors/synthesis (n=8), GABA receptors/synthesis (n=15), glial-enriched proteins (n-7), glutamate receptors/interacting proteins (n=24), phosphatases/kinases (n=21), neuropeptides (15), neurotrophins/neurotrophin receptors (n=12), synaptic/vesicular proteins (n=16), potassium/sodium channels (n=14), and others (n=6).

B. cDNAs that have a significantly higher hybridization signal intensity following 3′ TC RNA amplification versus 5′ TC RNA amplification (n=7) include: fos B, GluR3, KA2, Kv 1.2, NF-M, NF-H, and nestin.

C. cDNAs that have a significantly higher hybridization signal intensity following 5′ TC RNA amplification versus 3′ TC RNA amplification (n=6) include: αCAMKII, D2, GABA Aα1, GABA Aγ3, nAch rα1, and nAch rα7.

In contrast, the nicotinic acetylchoilne receptor subunits nAchr α1 and nAchr α7 display a relative increase in the 5′ TC amplification versus the 3′ TC RNA amplification. Therefore, hybridization signal intensity of individual genes and/or cDNAs/ESTs can vary between 3′ and 5′ TC RNA amplification, yet total populations of mRNAs have similar expression levels, indicating relatively equivalent signal detection efficiency.

Mechanism of TC primer annealing to 5′ regions of transcripts. In one specific embodiment of the present invention, there is a mechanism for the ability of the TC primer to anneal preferentially to the 5′ regions of transcripts, which was investigated using cloning to evaluate the 5′ regions of genes from a variety of brain tissue sources, including post mortem human brains and mouse brains. In a specific embodiment, the TC primer, with its span of C's (or G's) anneals preferentially within CpG islands. CpG islands are nonrethylated GC-rich regions of the genome that tend to include the 5′ end of genes. Estimates suggest that upwards of 60% of all human genes are located near CpG islands (Antequera et al., 1993; Cross et al., 1999). By annealing to regions that contain the 5′ regions of genes, the TC primer potential yields the highest likelihood of amplifying the overall population of genes, and accounts for the large transcript lengths following TC RNA amplification (FIGS. 9 and 10) and high sensitivity and hybridization signal intensity using cDNA arrays (FIG. 11).

Thus, as illustrated in this example, gene profiling is a powerful tool to examine the expression of multiple genes simultaneously. This paradigm can provide valuable insight into the pathophysiology of disease, tools for diagnosis, and guidance for the development of new pharmacotherapeutic interventions. However, one significant obstacle for the most effective application of gene profiling technology is the relative difficulty in utilizing small samples for subsequent downstream genetic analysis. The development of techniques such as laser capture microdissection (Emmert-Buck et al., 1996; Bonner et al., 1997) and single cell microaspiration (Ginsberg et al., 2001; Hemby et al., 2001) has allowed for the accession of minute amounts of starting materials including single cells as well as clusters of homogeneous cells in vitro and in vivo. However, an RNA amplification procedure is requisite to generate significant hybridization signal intensity for cDNA microarray platforms. PCR is not suitable for this application because exponential amplification cannot preserve optimally the quantitative relationships between the expressed genes, a parameter that is critical for gene profiling. The TC RNA amplification method is a protocol that meets both requirements of amplifying genetic signals as well as preserving the quantitative relationships between expressed genes. Essentially, the TC method amplifies genetic signals stepwise through in vitro RNA transcription. Therefore, transcripts can be amplified in linear fashion, preserving initial quantitative relationship(s) between the amplified genes. Compared to conventional RNA amplification methodologies, the TC method is more robust (approximately 3.5-4 fold stronger signal intensity) and significantly less laborious (the procedure takes approximately two days to complete).

A critical component of the TC RNA amplification method is the highly efficient second strand cDNA synthesis. Traditionally, this step is inefficient when the 5′ sequence of the first strand cDNA is not known. Under these conditions, a sequence-specific primer can not be generated to prime the second strand synthesis. Therefore, the generation of non-sequence specific primers by either self-priming or replacement strategies have been employed. In contrast, the TC method can attach an oligonucleotide primer of known sequence to 3′ of synthesized the first strand cDNA, thus providing a specific sequence platform for the priming of the second strand synthesis. As with the majority of mRNA amplification procedures, the first strand synthesis of the TC method is primed by a poly d(T) oligonucleotide primer. Following reverse transcription, along with the presence of the second (TC) primer, however, the reverse transcriptase continues DNA synthesis using the second primer as template. Therefore, the synthesized first strand cDNA will have a short stretch of oligonucleotides at the 3′ end that are complementary to the second (TC) primer. This paradigm enables the knowledge of the overhang 3′ sequence for first strand cDNA (at the 5′ end), thus a specific oligonucleotide acts to prime the synthesis of the second strand cDNA. Essential structural requirements of the second (TC) primer include a short stretch of cytosines or guanosines at the 3′ of the second primer. Replacement of the cytosines and guanosines with adenines or thymidines vastly diminishes the terminal continuation effect of the second primer.

In specific embodiments, the second primer has to base pair with the complementary C's or G's at the termination site of the reverse transcription reaction in order to provide a short template for DNA synthesis to continue. Several potential locations have been implicated for this complementary interaction to occur. For example, the reverse transcriptase reaction will add a few d(C)'s nonspecifically at the end of mRNA template. It has been observed that both d(C)'s and d(G)'s are added by reverse transcriptase activity that may base pair with the TC primer oligonucleotide sequence. Based upon the present results, however, a short stretch of C's and G's in mRNAs can also base pair with the G's and C's at the 3′ end of the second primer, thus providing a continuous template platform for reverse transcription under the appropriate conditions. Short regions of CG-rich CpG islands are prevalent at the 5′ region of approximately 60% of all human genes, and are found at a significantly less frequency (CpGs are 25% less frequent than predicted) throughout the rest of the genome (Antequera et al., 1993; Cross et al., 1999 ; Bird et al., 1987). CpG islands may represent a site whereby TC primers preferentially anneal, and would explain the long transcripts that are synthesized during the RNA amplification procedure. Further, replacement of G's or C's with A's or T's will almost completely abolish the efficacy of the TC primer. In addition, random base pairing of A's and T's with complementary T's and A's in mRNAs may interrupt a proper reverse transcription process that is essential for generation of the first strand cDNA.

The present series of results are primarily from brain tissues accrued from post mortem human samples and animal models of neurodegeneration. The brain is an obvious site for single cell RNA exploratory studies, due to the plethora of cell types and intricate connectivity of regions. The TC RNA amplification methodology, however, has much broader applications. Virtually an in vivo or in vitro setting can be employed for TC RNA amplification and subsequent downstream genetic analysis. Disciplines include, but are not restricted to, cancer biology, development, musculoskeletal, and a myriad of other sources of RNA. Current tissue sources include human, monkey, rat, and mouse tissues, and other sources are being investigated. The requirement appears to be polyadenylation on the 3′ end (no different than standard RNA amplification and a stretch of C's or G's on the 5′ region (either through CpG islands or other structures).

Thus, TC RNA amplification provides a technical means to amplify minute amounts of mRNAs for subsequent microarray or proteomic-based analyses. Conceivably, the downstream applications of synthesized RNA are expanded and the direction of RNA can be chosen according to the need. For example, antisense orientation may be selected for plasmid-designed cDNA microarray analysis, whereas a sense orientation may be selected for library construction or transcription for downstream proteomic applications and oligonucleotide-based microarray platforms.

### Example 8

### cDNA Microarray Analysis of Single Mouse Dentate Gyrus Granule Cells using Terminal Continuation for RNA Amplification

In this example, the efficiency of terminal continuation for downstream RNA amplification allowed for the study of individual neuron gene expression with relation to synaptic plasticity and neuronal remodeling following injury. Two paradigm methods were utilized in this example to cause synaptic and dendritic reorganization of adult mouse dentate gyrus granule cells: a unilateral perforant path (PP) transection and an intracerebral injection of kainate (KA).

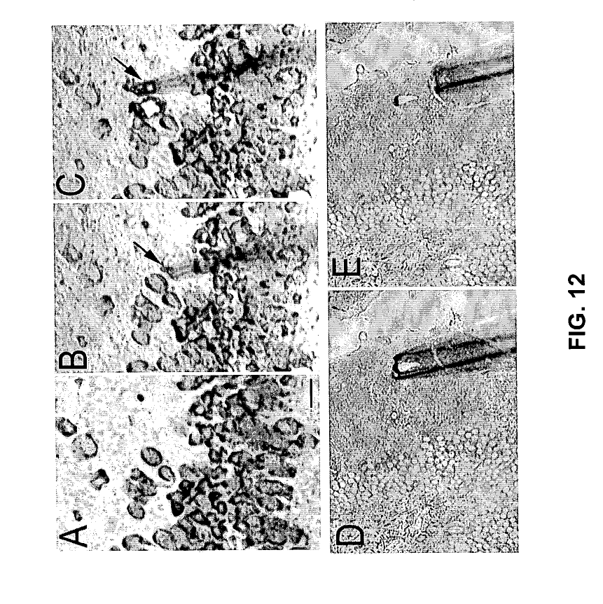

After injury through PP or KA, RNA was isolated at a short term after injury (1-5 days post-lesion) and at a long term after injury (10-90 days post-lesion), and after no injury (control). PP or KA was performed on adult C57BL/6 mice. Histology was conducted on brain sections of these mice to identify mouse dentate gyrus granule cells that have undergone synaptic and dendritic reorganization. Single cells were microdissected from the tissue section slides using single cell microdissection (FIG. 6). The cells of interest were identified through microscopy and recovered through a microaspiration device (FIG. 12). RNA was subsequently isolated using standard techniques. Single cell RNA was then amplified using the terminal continuation method (FIG. 3).

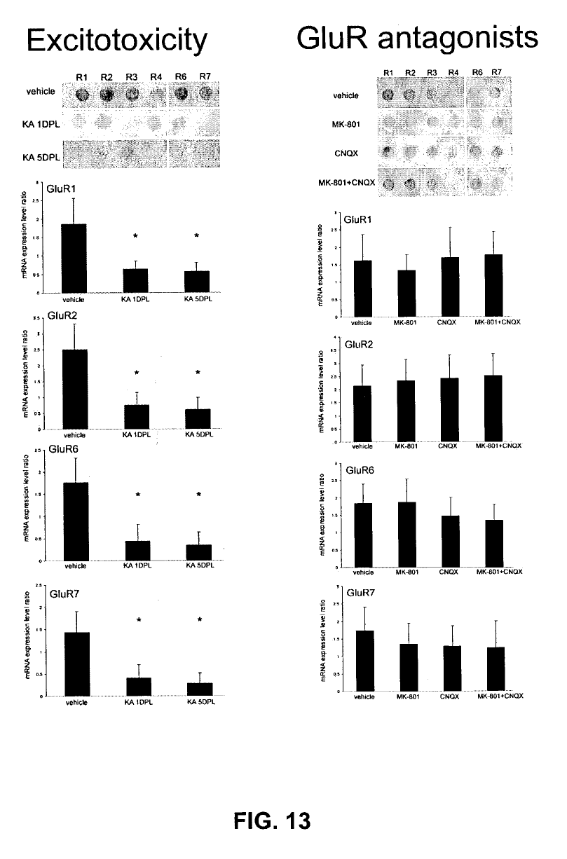

Amplified RNA was used to generate cDNA microarray probes to screen high-density (˜8,400 ESTs) and custom-designed (>225 cDNAs) cDNA array platforms. The results in FIG. 13 indicate a significant downregulation of GluR1, GluR2, GluR6 and GluR7 receptor subunits following both PP transections and KA injections. These expression profiles may provide early biomarkers for synaptic and dendritic changes and reveal novel targets for pharmacotherapeutic intervention.

### Example 9

Single Cell and Regional cDNA Microarray Analysis of Cholinergic Basal Forebrain Neurons using Terminal Continuation for RNA Amplification

The invention of terminal continuation allows the combination of precise tissue microdissection, RNA amplification, and expression profiling to test hypotheses that are difficult to attempt by assessing single genes or proteins in larger amounts of starting material. In this example, the principal goal was to utilize expression profiling methods to evaluate gene regulation in vulnerable cell types early in the pathogenesis of Alzheimer's disease (AD) for pharmacotherapeutic intervention.

RNA was isolated from individual neurons harvested from the various subfields of the cholinergic basal forebrain (CBF). Cholinergic neurons from the subfields of the CBF was obtained postmortem from subjects either with no cognitive impairment (NCI) or with Alzheimer's disease (AD).

Cholinergic basal forebrain tissues were sectioned and fixed. Histological stains were conducted to observe sections microscopically to identify a cell or cells/regions of interest. Individual cholinergic neurons were thus identified, and isolated using the single cell microdissection cell aspiration method (FIG. 12).

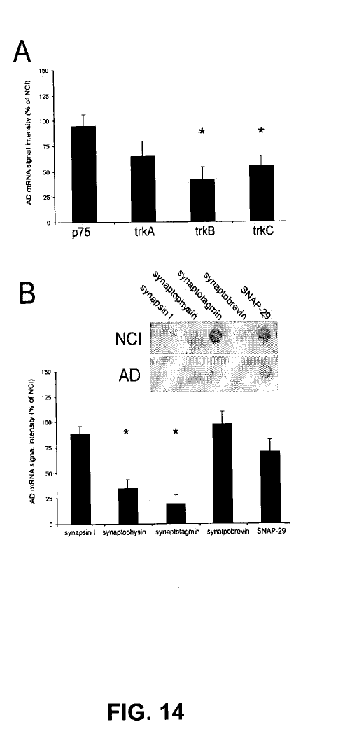

RNA was amplified using the terminal continuation method (FIG. 3), and cDNA was subsequently synthesized in sufficient amounts using the amplified RNA as templates. Resultant cDNA from NCI subjects or AD subjects was used to generate custom-designed cDNA microarrays and probes for use with these microarrays. Such single cell analyses revealed alterations between NCI and AD subjects in relevant classes of transcripts including neurotrophin receptors, protein phosphatases and kinases, and synaptic markers (synapsin I, synaptophysin, synaptotagmin, synaptobrevin, SNAP-29, FIG. 14). In FIG. 14, the expression of synaptic markers was significantly reduced in cells recovered from subjects with Alzheimer's disease. These studies provided novel regional and single cell molecular fingerprints of vulnerable cells to neurodegeneration that may help to define early biomarkers and mechanisms of pathogenesis of AD and related dementia disorders.

### Example 10

### Profile of Gene Expression from Microdysgenic Cortical Neurons using Terminal Continuation RNA Amplification

Intractable seizures during childhood are frequently associated with cellular neuropathology. For example, neuromigrational abnormalities resulting in microdysgenesis are a common feature. A gene expression profile of microdysgenic neurons was created by use of terminal continuation based RNA amplification, microdissection and microarray analysis.

Microdysgenic neurons were obtained from a biopsy resection of the temporal cortex of a child with an intractable seizure disorder. The epileptic focus was surgically removed to control the seizures. Neuropathological observation of the resected tissue indicated extensive microdysgenesis within the temporal cortex in addition to a ganglionglioma.

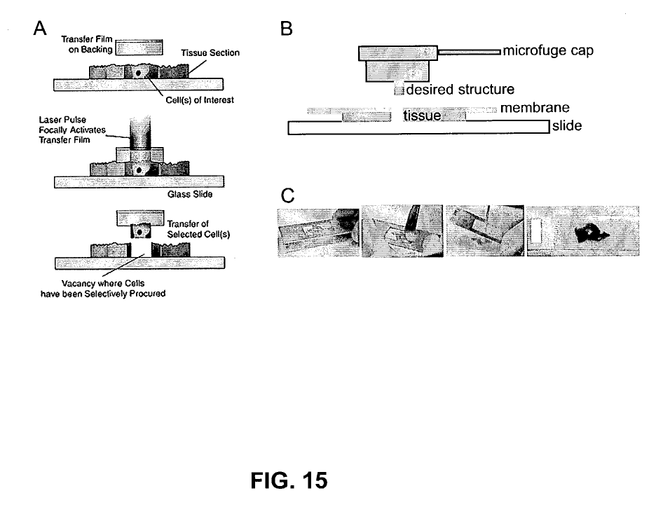

Tissue from the dysgenic temporal cortex was removed in accordance with standard approved surgical procedures and processed for further neuropathological analysis. Thin paraffin sections were immunohistochemically stained with anti-NeuN antibodies to reveal the location of neurons. Abnormal neurons that appeared to be in direct contact with each other (“clustered neurons”) were isolated using the laser capture microdissection cell aspiration method (FIG. 6). Laser capture microdissection (LCM) uses a microscopy based instrumentation (FIG. 15). Essentially, cells of interest are identified using the microscopy part of the LCM instrument, and then these cells are transferred either to a microfuge cap or membrane (section B, FIG. 12) through the use of a laser, either infrared or ultraviolet (section A, FIG. 12). Normal neurons (non-clustered) were also isolated from surrounding and adjacent cortical areas for use as controls.

Terminal continuation based RNA amplification was performed in combination with custom-designed cDNA arrays for the simultaneous analysis of over 200 genes relevant towards neurodegeneration and brain function. Five pairs of “clustered” neurons and 5 pairs of “non-clustered” control neurons were processed for analysis with 96 blot gene arrays. As expected, a dynamic range of gene expression levels was observed across the 207 genes studied. Preliminary results indicated that several subsets of genes from distinct cellular pathways were differentially regulated between clustered and non-clustered cells. These data provided an initial molecular fingerprint of microdysgenetic cells from a human biopsy sample that will be relevant towards the study of the molecular pathophysiology of migrational and seizure disorders.

### Example 11

### Gene Expression Analysis from Adjacent Tissue Stained Differentailly

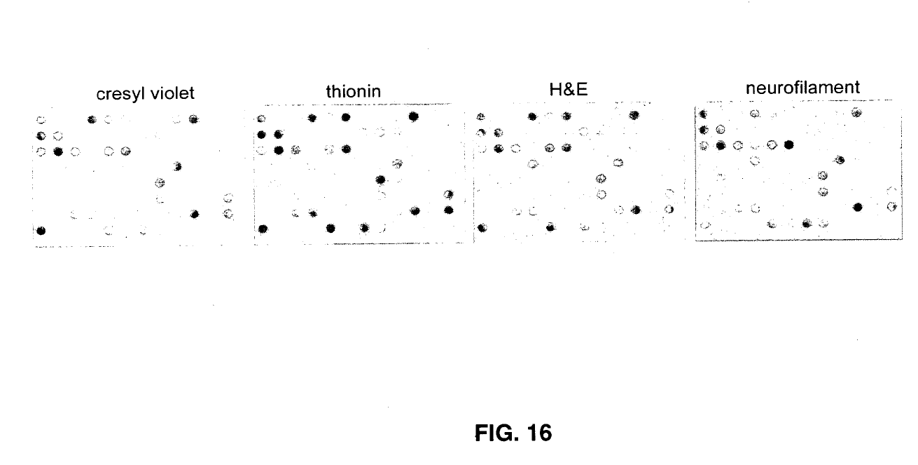

FIG. 16 illustrates gene expression profiles from serial adjacent 6 μm-thick tissue sections (paraffin embedded, 70% ethanol buffered with 150 mM sodium chloride) from the same human hippocampus stained with different stains. All of the arrays were synthesized concomitantly, and the RNA amplification was performed simultaneously. No apparent differences are detectable using tissue aspirated from the different staining conditions.

Nissl stain: cresyl violet:

1. Deparaffinize slides and hydrate to ddH2O (xylenes 2×5 min, 100% EtOH 2×1 min, 95%, 80%, 70%, ddH2O 1 min each).

2. Immerse sections in filtered 1% cresyl violet for 2 min.

3. Differentiate sections in 95% EtOH until only cell bodies are visualized and background is low. Check background level of each slide under microscope before step 4.

4. Immerse sections in 100% EtOH for 30 seconds.

Hemotoxylin and eosin stain:

1. Deparaffinize slides and hydrate to ddH₂O (xylenes 2×5 min, 100% EtOH 2×1 min, 95%, 80%, 70%, ddH₂O 1 min each).

2. Immerse sections in filtered undiluted Hematoxylin (Gills #2) 1 min and rinse in ddH₂O.

3. Immerse sections in 1% lithium carbonate for approximately 30 seconds and rinse in ddH₂O.

4. Immerse sections in 1% eosin solution for 1 minute, differentiate in 80% EtOH and rinse in ddH₂O.

Acridine orange stain:

1. Deparaffinize slides and hydrate to ddH2O (xylenes 2×5 min, 100% EtOH 2×1 min, 95%, 80%, 70%, ddH2O 1 min each).

2. Immerse sections in 0.2 M dibasic sodium phosphate/0.1 M citric acid (SC buffer; pH 4.0) solution for 5 min.

3. Immerse sections in acridine orange solution (10 μg/ml in SC buffer) for 15 min.

4. Rinse the sections SC buffer (3×1 min) and immerse sections in 50% ethanol in phosphate-buffered saline (PBS; 0.12 M; pH 7.4) for 2 min.

In FIG. 16, Nissl stains include cresyl violet and thionin, and H&E stands for hemotoxylin and eosin The neurofilament section is stained with an antibody against neurofilaments by standard methods in the art.

### Example 12

### Comparison of Total Signal Intensity using Different Stains

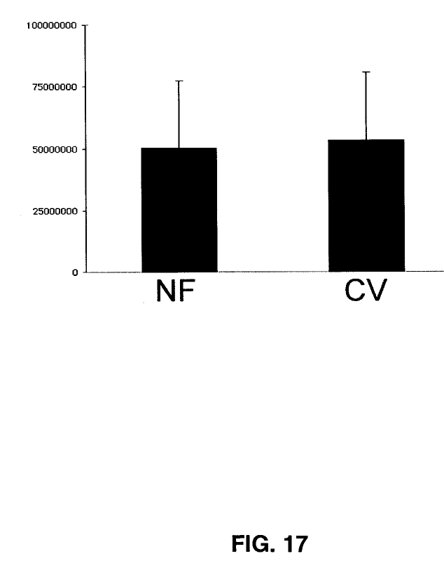

The methods of the present invention were utilized to compare adjacent sections stained with an antibody (neurofilament, NF) and a histological stain (cresyl violet, CV) (FIG. 17). Total hybridization signal intensity on the array (220 cDNAs) is presented with means and standard deviations. No significant differences are seen in antibody versus histological stained sections, particularly given that cresyl violet did not render the RNA inaccessible by, for example, the primer.

Arrays are generated using high-density nitrocellulose, 96 well slot blot apparatus, and a 12-channel micropipettor. One microgram of linearized cDNA purified from plasmid preparations is adhered to nitrocellulose membranes in a final volume of 50 μl. cDNA clones/ESTs (approximately 220) corresponding to specific subgroups include: glutamate receptors/transporters (n=22), glutamate receptor interacting proteins (n=6), synaptic/vesicular proteins (n=10), immediate early/cell death genes (n=19), GABA synthesis/receptors/transporters (n=17), cytoskeletal elements (n=15), protein phosphatases/kinases (n=23), neurotrophins/neurotrophin receptors (n=12), AD-linked genes (n=12), calcium binding proteins/calcium channels (n=7), glial/microglial enriched markers (n=6), monoamine synthesis/transporters (n=7), dopamine receptors/transporters (n=6), neuropeptides/neuropeptide receptors (n=15), acetylcholine synthesis/receptors (n=15), potassium/sodium channels (n=11), positive controls (n=2), negative controls (n=2), and others (n=15). ). Each cDNA/EST on the custom-deigned cDNA arrays is verified by restriction digestion and sequence analysis. Arrays are prehybridized (12 hours) and hybridized (48 hours) in a solution consisting of 6×SSPE, 5×Denhardt's solution, 50% formamide, 0.1% sodium dodecyl sulfate (SDS), and denatured salmon sperm DNA (200 μg/ml) at 42° C. in a rotisserie oven. Following hybridization, arrays are washed sequentially with 2×SSC/0.1% SDS, 0.5×SSC/0.1% SDS and 0.1×SSC/0.1% SDS for 20 min each at 42° C. aRNA hybridization signal intensity is detected by phosphor imaging. The hybridization signal intensity of the empty vector pBs (double spotted on the arrays) serves to identify background. The specific signal intensity (minus background) of aRNA bound to each linearized cDNA is expressed as a ratio of the total hybridization signal intensity of the array, thereby minimizing variations due to differences in the specific activity of the probe and the absolute quantity of probe present. Data analyzed in this manner does not allow the absolute quantitation of mRNA levels, but generates an expression profile of the relative changes in mRNA levels. Relative changes in individual mRNAs are analyzed using ANOVA with post-hoc analysis (Newman-Keuls test) for individual comparisons.

### REFERENCES

The following references, to the extent that they provide exemplary procedural or other details supplementary to those set forth herein, are specifically incorporated herein by reference.

### PATENTS

European Application No. 320 308

European Application No. 329 822

PCT Application No. PCT/US87/00880

PCT Application No. PCT/US89/01025

PCT Application WO 88/10315

PCT Application WO 89/06700

PCT Application WO 90/07641

U.S. Pat. No. 4,683,195

U.S. Pat. No. 4,883,750

U.S. Pat. No. 4,946,773

U.S. Pat. No. 5,279,721

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,483

U.S. Pat. No. 5,849,487

U.S. Pat. No. 5,849,497

U.S. Pat. No. 5,849,546

U.S. Pat. No. 5,849,547

U.S. Pat. No. 5,851,770

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,337

U.S. Pat. No. 5,866,366

U.S. Pat. No. 5,882,864

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,630

U.S. Pat. No. 5,922,574

U.S. Pat. No. 5,925,517

U.S. Pat. No. 5,925,525

U.S. Pat. No. 5,928,862

U.S. Pat. No. 5,928,869

U.S. Pat. No. 5,928,870

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,825

U.S. Pat. No. 5,939,291

U.S. Pat. No. 5,942,391

U.S. Pat. No. 4,683,202

U.S. Pat. No. 4,800,159

U.S. Pat. No. 5,849,486

U.S. Pat. No. 5,851,772

U.S. Pat. No. 5,900,481

U.S. Pat. No. 5,919,626

GB Application No. 2 202 328

### PUBLICATIONS

Bellus, 1994.

Eberwine, J., Crino, P. and Dichter, M., Single-cell mRNA amplification: implications for basic and clinical neuroscience, The Neuroscientist, 1 (1995) 200-211.

Eberwine, J., Yeh, H., Miyashiro, K., Cao, Y., Nair, S., Finnell, R., Zettel, M. and Coleman, P., Analysis of gene expression in single live neurons, Proc. Natl. Acad. Sci. USA, 89 (1992) 3010-3014.

Florell S R, Coffin C M, Holden J A, Zimmermann J W, Gerwels J W, Summers B K, Jones D A, Leachman S A. 2001. Preservation of RNA for flnctional genomic studies: a multidisciplinary tumor bank protocol. Mod. Pathol. 14(2): 116-128.

Frohman, In: PCR PROTOCOLS. A GUIDE TO METHODS AND APPLICATIONS, Academic Press, N.Y., 1990.

Ginsberg, S. D., Expression profile of dentate gyros granule cells following perforant path lesions in adult mice using gene microarrays, Proc. Soc. Neurosci., 26 (2000) 607.

Ginsberg, S. D., Crino, P. B., Hemby, S. E., Weingarten, J. A., Lee, V. M. -Y., Eberwine, J. H. and Trojanowski, J. Q., Predominance of neuronal mRNAs in individual Alzheimer's disease senile plaques, Ann. Neurol., 45 (1999) 174-181.

Ginsberg, S. D., Hemby, S. E., Lee, V. M. -Y., Eberwine, J. H. and Trojanowski, J. Q., Expression profile of transcripts in Alzheimer's disease tangle-bearing CA1 neurons, Ann. Neurol., 48 (2000) 77-87.

Ginsberg, S. D., Schmidt, M. L., Crino, P. B., Eberwine, J. H., Lee, V. M. -Y. and Trojanowski, J. Q., Molecular pathology of Alzheimer's disease and related disorders. In A. Peters and J. H. Morrison (Eds.), Cerebral Cortex, vol. 14. Neurodegenerative and Age-related Changes in Structure and Function of Cerebral Cortex, Kluwer Academic/Plenum, New York, 1999, pp. 603-653.

Hemby, S. E., Ginsberg, S. D., Brunk, B., Arnold, S. E., Overton, C., Trojanowski, J. Q. and Eberwine, J. H., A mRNA expression profile for schizophrenia: single-neuron transcription patterns from the entorhinal cortex, Submitted for publication (2001).

Innis et al., “DNA sequencing with Thermus aquaticus DNA polymerase and direct sequencing of polymerase chain reaction-amplified DNA,” Proc Natl Acad Sci U S A. 85(24):9436-9440, 1988.

Kwoh et al., “Transcription-based amplification system and detection of amplified human immunodeficiency virus type 1 with a bead-based sandwich hybridization format, Proc Natl Acad Sci U S A. 86(4):1173-1177, 1989.

Lillie, R. D. (1969), Conn's Biological Stains, 8th ed., Williams and Wilkins Company, Baltimore, Md.

Luzzi V, Holtschlag V, Watson M A. 2001. Expression profiling of ductal carcinoma in situ by laser capture microdissection and high-density oligonucleotide arrays. Am J Pathol 158:2005-2010.

Merck Index, 12 ed., Susan Budavari, (ed.), Merck & Co., NJ, USA.

Mufson, E. J. and Ginsberg, S.D., Expression profile of cholinergic basal forebrain neurons in mild Alzheimer's disease using gene microarrays, Proc. Soc. Neurosci., 26 (2000) 232.

Ohara et al., “One-sided polymerase chain reaction: the amplification of cDNA,”

Phillips J, Eberwine J H. 1996. Antisense RNA Amplification: A Linear Amplification Method for Analyzing the mRNA Population from Single Living Cells. Methods Enzymol. Suppl. 10:283-288.

Saito K, Sullivan D, Haruna Y, Theise N D, Thung W N, Gerber M A. 1997. Detection of hepatitus C virus RNA sequences in hepatocellular carcinoma and its precursors by microdissection polymerase chain reaction. Arch Pathol Lab Med 121:400-403.

Sambrook et al., In: Molecular Cloning: A Laboratory Manual, Vol. 1, Cold Spring Harbor Laboratory Press, Cold Spring Harbor, N.Y., Ch. 7,7.19-17.29, 1989.

To M D, Done S J, Redston M, Andrulis I L. 1998. Analysis of mRNA from microdissected frozen tissue sections without RNA isolation. Am. J. Pathol. 153(1):47-51.

VanGelder, R., von Zastrow, M., Yool, A., Dement, W., Barchas, J. and Eberwine, J., Amplified RNA (aRNA) synthesized from limited quantities of heterogeneous cDNA, Proc. Natl. Acad. Sci. USA, 87 (1990) 1663-1667.

Walker et al., “Strand displacement amplification—an isothermal, in vitro DNA amplification technique,” Nucleic Acids Res. 20(7): 1691 -1696, 1992

It will be apparent to those skilled in the art that some modifications, such as modifications of sequences in first and second primers in the preferred protocol can lead to the expand applications of amplified RNA population, such as constructing subtractive cDNA libraries, and expression libraries. Therefore, it should be understood that the examples and embodiments described herein are for illustrative purposes only and that various modifications or changes in light thereof will be suggested to persons skilled in the art and are to be included within the spirit and purview of this application and the scope of the claims.

One skilled in the art readily appreciates that the patent invention is well adapted to carry out the objectives and obtain the ends and advantages mentioned as well as those inherent therein. Methods, procedures, techniques, and kits described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

We claim:

1. A method to amplify an RNA molecule, comprising:
obtaining said RNA molecule;
introducing to said mRNA molecule a first primer, wherein said first primer comprises a region that hybridizes under suitable conditions to a complementary region of said RNA molecule;
introducing to said RNA molecule and said first primer a second primer, wherein said second primer comprises at least one riboguanine at the 3′ end of said primer;
synthesizing a first complementary nucleic acid molecule to said RNA molecule by extending said first primer using reverse transcriptase under conditions wherein said synthesis results in there being more than one cytosine at the 3′ end of said first complementary nucleic acid molecule, wherein said synthesis results in an RNA-first complementary nucleic acid molecule hybrid comprising the first primer and its extension product bound to the second primer and the RNA;
removing said RNA molecule and said second primer from said hybrid;
synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and
transcribing at least one mRNA molecule from said first complementary nucleic acid molecule and second complementary nucleic acid molecule hybrid.

2. The method of claim 1, wherein said RNA molecule is an mRNA molecule.

3. The method of claim 1, wherein said RNA is a tRNA molecule.

4. The method of claim 1, wherein said RNA is a rRNA molecule.

5. The method of claim 1, wherein said RNA molecule is obtained from a plurality of RNA molecules.

6. The method of claim 5, wherein said plurality of RNA molecules comprises mRNA, tRNA, rRNA, or a combination thereof.

7. The method of claim 1, wherein said first primer further comprises a region comprising at least two poly(dT)s.

8. The method of claim 1, wherein said first primer is a short primer of random sequence.

9. The method of claim 1, wherein said first primer further comprises a region selected from the group consisting of a promoter region, a restriction enzyme digestion sequence, and a combination thereof.

10. The method of claim 1, wherein said first primer further comprises a promoter region.

11. The method of claim 10, wherein said promoter is a bacteriophage transcription promoter.

12. The method of claim 11, wherein said bacteriophage transcription promoter is selected from the group consisting of T7 RNA polymerase promoter, T3 RNA polymerase promoter, SP6 RNA polymerase promoter, and a recombinant promoter.

13. The method of claim 1, wherein said second primer comprises a random sequence at it 5′ end and at least one guanine, deoxyguanine, cytosine, or deoxycytosine at its 3′ end.

14. The method of claim 1, wherein said second primer comprises a random sequence at it 5′ end and at least one guanine or cytosine at its 3′ end.

15. The method of claim 13, wherein said second primer further comprises a region selected from the group consisting of a promoter region, a protein translation start region, a restriction enzyme digestion sequence, and a combination thereof.

16. The method of claim 1, wherein said second primer further comprises a promoter.

17. The method of claim 16, wherein said promoter is a bacteriophage transcription promoter.

18. The method of claim 17, wherein said bacteriophage transcription promoter is selected from the group consisting of T7 RNA polymerase promoter, T3 RNA polymerase promoter, SP6 RNA polymerase promoter, and a recombinant promoter.

19. The method of claim 1, wherein said reverse transcriptase is selected from the group consisting of Taq reverse transcriptase, Moloney Murine Leukemia Virus reverse transcriptase, Moloney Murine Leukemia Virus reverse transcriptase lacking RNAseH activity, Avian Myeloblastosis Virus reverse transcriptase, Avian Myeloblastosis Virus reverse transcriptase lacking RNAseH activity, human T-cell leukemia virus type I (HTLV-I), Rous-associated virus 2 (RAV2), bovine leukemia virus (BLV), Rous sarcoma virus (RSV), HIV-1 reverse transcriptase, TERT reverse transcriptase, and Tth reverse transcriptase.

20. The method of claim 1, wherein said method further comprises at least one step of reverse transcribing said mRNA molecule from said transcription step, wherein said reverse transcription results in generating at least one cDNA molecule.

21. The method of claim 20, wherein said reverse transcribing step is primed by at least one random primer.

22. The method of claim 20, wherein said reverse transcribing step is primed by a primer attached to said first complementary nucleic acid molecule, said second complementary nucleic acid molecule, or a combination thereof.

23. The method of claim 20, wherein said cDNA molecule comprises at least one promoter sequence.

24. The method of claim 23, wherein said promoter is a bacteriophage transcription promoter.

25. The method of claim 24, wherein said bacteriophage transcription promoter is selected from the group consisting of T7 RNA polymerase promoter, T3 RNA polymerase promoter, SP6 RNA polymerase promoter, and a recombinant promoter.

26. The method of claim 1, wherein said RNA is removed by RNAase digestion.

27. The method of claim 1, wherein said RNA is removed by RNAse digestion, by heating in solution comprising a low concentration of MgCl₂, or by a combination thereof.

28. A method to amplify an mRNA molecule, comprising:
obtaining said mRNA molecule;
introducing to said MrRNA molecule a first primer, wherein said first primer comprises:
at least two poly(dT)s; and
random sequences;
introducing to said mRNA molecule and said first primer a second primer, wherein said second primer comprises:
at least one riboguanine at the 3′ end of said primer; and
a bacteriophage promoter sequence;
synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA;
removing said mRNA molecule and said second primer from said hybrid;
synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and
transcribing at least one mRNA molecule from said first complementary nucleic acid molecule and second complementary nucleic acid molecule hybrid.

29. A method to amplify an mRNA molecule, comprising:
obtaining said mRNA molecule;
introducing to said mRNA molecule a first primer, wherein said first primer comprises:
at least two poly(dT)s; and
a bacteriophage promoter sequence;
introducing to said mRNA molecule and said first primer a second primer, wherein said second primer comprises at least one riboguanine at the 3′ end of said primer;
synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA;
removing said mRNA molecule and said second primer from said hybrid;
introducing to said complementary nucleic acid molecule an oligo (dNTP) primer with substantially the same sequence as said second primer;
synthesizing a second complementary nucleic acid molecule to said first complementary nucleic acid molecule, wherein said synthesis results in a first complementary nucleic acid molecule and second complementary nucleic acid molecule hybrid; and
transcribing at least one mRNA molecule from said first complementary nucleic acid molecule and second complementary nucleic acid molecule hybrid, wherein said at least one mRNA molecule is an antisense mRNA.

30. A method to amplify an mRNA molecule, comprising:
obtaining said mRNA molecule;
introducing to said mRNA molecule a first primer, wherein said first primer comprises at least two poly(dT)s or a short primer of random sequence;
introducing to said mRNA molecule and said first primer a second primer, wherein said second primer comprises:
at least one riboguanine at the 3′ end of said primer; and
a bacteriophage promoter sequence;
synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA;
removing said mRNA molecule and said second primer from said hybrid;
introducing to said complementary nucleic acid molecule an oligo (dNTP) primer with substantially the same sequence as said second primer;
synthesizing a second complementary nucleic acid molecule to said first complementary nucleic acid molecule, wherein said synthesis results in a first complementary nucleic acid molecule and second complementary nucleic acid molecule hybrid; and
transcribing at least one mRNA molecule from said first complementary nucleic acid molecule and second complementary nucleic acid molecule hybrid, wherein said at least one mRNA molecule is a sense mRNA molecule.

31. A kit for amplifying an RNA molecule using the method of claim 1, wherein said kit is in a suitable container and comprises said first primer, said second primer, said third primer, or a combination thereof.

32. The kit of claim 31, wherein said first primer is a short primer of random sequences.

33. The kit of claim 31, wherein said first primer further comprises a region selected from the group consisting of a promoter, a restriction enzyme digestion sequence, and a combination thereof.

34. The kit of claim 31, wherein said second primer further comprises a region selected from the group consisting of a promoter, a restriction enzyme digestion sequence, and a combination thereof.

35. A method of providing a substrate for in vitro transcription, comprising:
obtaining said mRNA molecule;
introducing to said mRNA molecule a first primer, wherein said first primer comprises a region which anneals under suitable conditions to a complementary region of said mRNA molecule;
introducing to said mRNA molecule and said first primer a second primer, wherein said second primer comprises at least one riboguanine at the 3′ end of said primer;
synthesizing a first complementary nucleic acid molecule to said mRNA molecule by extending said first primer using reverse transcriptase under conditions wherein said synthesis results in there being more than one cytosine at the 3′ end of said first complementary nucleic acid molecule, wherein said synthesis results in an mRNA-first complementary nucleic acid molecule hybrid comprising the first primer and its extension product bound to the second primer and the mRNA;
removing said mRNA molecule and said second primer from said hybrid;
synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and
transcribing at least one mRNA molecule from said first complementary nucleic acid molecule and second complementary nucleic acid molecule hybrid.

36. A method of detecting an RNA from a histologically-stained cell, comprising:
obtaining the cell;
extracting RNA from the cell; and
amplifying the RNA.

37. The method of claim 36, wherein the cell is in a tissue.

38. A method of detecting an RNA from a cell, comprising:
obtaining the cell;
histologically staining the cell;
extracting RNA from the cell; and
amplifying the RNA.

39. The method of claim 38, wherein the cell is in a tissue.

40. The method of claim 39, wherein the tissue is fresh tissue.

41. The method of claim 39, wherein the tissue is fixed tissue.

42. The method of claim 41, wherein the tissue is fixed by acetone, aldehyde derivatives, ethanol, or combinations thereof.

43. The method of claim 36 or 38, wherein said cell is from a physiological body fluid, a pathological exudate, or a pathological transudate.

44. The method of claim 43, wherein the physiological body fluid is blood, cerebrospinal fluid, urine, sweat, semen, or saliva.

45. The method of claim 38, wherein the cells are in blood, bone marrow, cerebrospinal fluid, or any other physiological body fluids or any pathological exudates or transudates.

46. The method of claim 36 or 38, wherein said cell is from bone marrow.

47. The method of claim 36 or 38, wherein said cell is from in vitro cultured cells.

48. The method of claim 36 or 38, wherein the histological stain identifies cellular structures.

49. The method of claim 48, wherein said cellular structures are mitochondria, centrioles, rough endoplasmic reticulum, smooth endoplasmic reticulum, peroxisomes, endosomes, lysosomes, vesicles, Golgi apparatus, nucleus, cytoplasm, or a combination thereof.

50. The method of claim 37 or 39, wherein the histological stain identifies tissue structures.

51. The method of claim 50, wherein said tissue structures are structures of lamina, matrix, or a combination thereof.

52. The method of claim 36 or 38, wherein the histological stain is Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocarmine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Ingrain blue, Ingrain blue 1, Ingrain yellow 1, INT, Kermes, Kermesic acid, Kemechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

53. The method of claim 37 or 39, wherein the extracting step further comprises dissection of the cell from the tissue.

54. The method of claim 53, wherein the dissection is from a micropipette on a micromanipulator or by laser capture microdissection.

55. The method of claim 36, wherein the amplifying step further comprises synthesis of cDNA from the RNA.

56. The method of claim 55, wherein the synthesis of cDNA further comprises synthesizing the cDNA by reverse transcriptase with an oligonucleotide that binds the RNA.

57. The method of claim 36, wherein the RNA amplification method is in vitro transcription.

58. The method of claim 36, wherein the amplification is by a method which comprises:
introducing to said RNA molecule a first primer, wherein said first primer comprises a region that hybridizes under suitable conditions to a complementary region of said RNA molecule;
introducing to said RNA molecule and said first primer a second primer, wherein said second primer comprises at least one riboguanine at the 3′ end of said primer;
synthesizing a first complementary nucleic acid molecule to said RNA molecule by extending said first primer using reverse transcriptase under conditions wherein said synthesis results in there being more than one cytosine at the 3′ end of said first complementary nucleic acid molecule, wherein said synthesis results in an RNA-first complementary nucleic acid molecule hybrid comprising the first primer and its extension product bound to the second primer and the RNA;
removing said RNA molecule and said second primer from said hybrid;
synthesizing a second complementary nucleic acid molecule to said first complementary nucleic acid molecule, wherein said synthesis results in a first complementary nucleic acid molecule and second complementary nucleic acid molecule hybrid, wherein the hybrid further comprises both a third primer with a sequence substantially similar to the second primer and an extension product of the third primer bound to the first complementary nucleic acid molecule; and
transcribing at least one mRNA molecule from said first complementary nucleic acid molecule and second complementary nucleic acid molecule hybrid.

59. A kit, housed in a suitable container, for the detection of RNA from a cell in a histologically-stained tissue, comprising dye/histological stain, RNA extraction reagent, RNA precipitation carrier, oligo (dT) primer, reverse transcriptase, DNA polymerase, RNA polymerase, RNAse inactivating agent, terminal continuation oligonucleotide, dNTPs, NTPs, or a combination thereof.

60. The kit of claim 59, wherein the RNA polymerase is T7 RNA polymerase, T3 RNA polymerase, or SP6 RNA polymerase.

61. The kit of claim 59, wherein the kit further comprises a vector, a ligase, or a combination thereof.

62. The kit of claim 59, wherein the dye/histological stain is Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocarmine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fiuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulfate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

63. A method of incorporating a nucleic acid sequence to a 3′ region of a synthesized nucleic acid strand comprising:
incubating a target nucleic acid strand with a terminal continuation oligonucleotide, and a first strand synthesis primer which is complementary to a region at the 3′ end or a region upstream of the 3′ end of the target nucleic acid strand under conditions that facilitate hybridization of the first strand synthesis primer to the target nucleic acid strand; and
extending the primer, wherein the extending is carried out with a polymerase such that extension synthesizes a nucleic acid strand comprising the first strand synthesis primer, a complementary sequence of the target nucleic acid strand, and a complement of the terminal continuation oligonucleotide.

64. The method of claim 63 wherein the terminal continuation oligonucleotide contains at least one guanine, deoxyguanine, cytosine, or deoxycytosine at the 3′ end of the terminal continuation oligonucleotide.

65. The method of claim 63 wherein the target nucleic acid strand is RNA and the polymerase is reverse-transcriptase, such that the nucleic acid synthesized in the extending step is a first strand cDNA comprising the first strand synthesis primer, a complement of the target nucleic acid strand, and a complement of the terminal continuation oligonucleotide at the 3′ end.

66. The method of claim 65 wherein the RNA is mRNA.

67. The method of claim 65 wherein the first strand synthesis primer comprises at least two thymidine residues at its 3′ end.

68. The method of claim 65 wherein the first strand synthesis primer comprises a random hexamer sequence of nucleic acid.

69. The method of claim 65 wherein the terminal continuation oligonucleotide comprises at least two nucleotides selected from a group consisting of guanine, deoxyguanine, cytosine or deoxycytosine bases.

70. The method of claim 65 comprising the additional steps:
incubating the first strand cDNA with the terminal continuation oligonucleotide under conditions that facilitate hybridization of the terminal continuation oligonucleotide to the first strand cDNA; and
extending the terminal continuation oligonucleotide, wherein said extending is carried out with a DNA polymerase such that extension synthesizes a second strand cDNA comprising the sequence of the terminal continuation oligonucleotide and a complementary sequence of the first strand cDNA.

71. The method of claim 70 wherein the DNA polymerase is Taq polymerase.

72. The method of claim 70 wherein the first strand synthesis primer comprises a transcriptional promoter sequence.

73. The method of claim 70 wherein the terminal continuation oligonucleotide comprises a transcriptional promoter sequence and at least one guanine, deoxyguanine, cytosine, or deoxycytosine at the 3′ end of the terminal continuation oligonucleotide.

74. The method of claim 70, wherein the terminal continuation oligonucleotide comprises a transcriptional promoter sequence and at least one guanine or cytosine at the 3′ end of the terminal continuation oligonucleotide.

75. The method of claim 74 comprising the additional steps:
incubating the second strand cDNA with a RNA polymerase capable of binding to the transcriptional promoter sequence; and
transcribing the second strand cDNA wherein the transcribing synthesizes a RNA transcript complementary in sequence to the second strand cDNA.

76. The method of claim 73 comprising the additional steps:
incubating the first strand cDNA with a RNA polymerase capable of binding to the transcriptional promoter sequence; and
transcribing the first strand cDNA wherein the transcribing synthesizes a RNA transcript complementary in sequence to the first strand cDNA.

77. The method of claim 70 wherein the first strand synthesis primer comprises a transcriptional promoter sequence and wherein the terminal continuation oligonucleotide comprises at least one guanine, deoxyguanine, cytosine, or deoxycytosine at its 3′ end and a transcriptional promoter sequence different from the transcriptional promoter sequence in the first strand synthesis primer.

78. The method of claim 77 comprising the additional steps:
incubating the first strand cDNA with a RNA polymerase capable of binding to the transcriptional promoter sequence located on the first strand cDNA;
transcribing the first strand cDNA wherein the transcribing synthesizes a RNA transcript complementary in sequence to the first strand cDNA;
incubating the second cDNA strand with a RNA polymerase capable of binding to the transcriptional promoter sequence located on the second strand cDNA; and
transcribing the second strand cDNA wherein the transcribing synthesizes a RNA transcript complementary in sequence to the second strand cDNA.

79. The method of claim 75 or 78 wherein the synthesized RNA transcripts are used as templates for in vitro translation.
